SEQUENCE LISTING (45 sequences)

SEQ ID NO: 1 - protein, 676 aa, homo sapiens
MAAAKVALTKRADPAELRTIFLKYASIEKNGEFFMSPNDFVTRYLNIFGESQPNPKTVELLSGVVDQTKDGLISFQEFVAFESVLCAPDALFMVAFQLFDKAGKGEVTFEDVKQVFGQTTIHQHIPFNWDSEFVQLHFGKERKRHLTYAEFTQFLLEIQLEHAKQAFVQRDNARTGRVTAIDFRDIMVTIRPHVLTPFVEECLVAAAGGTTSHQVSFSYFNGFNSLLNNMELIRKIYSTLAGTRKDVEVTKEEFVLAAQKFGQVTPMEVDILFQLADLYEPRGRMTLADIERIAPLEEGTLPFNLAEAQRQQKASGDSARPVLLQVAESAYRFGLGSVAGAVGATAVYPIDLVKTRMQNQRSTGSFVGELMYKNSFDCFKKVLRYEGFFGLYRGLLPQLLGVAPEKAIKLTVNDFVRDKFMHKDGSVPLAAEILAGGCAGGSQVIFTNPLEIVKIRLQVAGEITTGPRVSALSVVRDLGFFGIYKGAKACFLRDIPFSAIYFPCYAHVKASFANEDGQVSPGSLLLAGAIAGMPAASLVTPADVIKTRLQVAARAGQTTYSGVIDCFRKILREEGPKALWKGAGARVFRSSPQFGVTLLTYELLQRWFYIDFGGVKPMGSEPVPKSRINLPAPNPDHVGGYKLAVATFAGIENKFGLYLPLFKPSVSTSKAIGGGP

SEQ ID NO: 2 - DNA, 3207 bp, homo sapiens
aatgggcgggcagcatccacatgacccgcgccggcgggagggcgtggggaggcaggccaggaacgcacgctgcctggccgtatcgccgcccccaccgccgccgccgccgggactagaagtgagccgcccgggtcccaaacgccagccagccagtcagtgggtcccgcagtcgcccgcaaccggggcgaatcatggcggccgccaaggtggctttaaccaagagagcagatccagctgagcttagaacaatatttttgaagtatgcaagcattgagaaaaacggtgaatttttcatgtcccccaatgactttgtcactcgatacttgaacatttttggagaaagccagcctaatccaaagactgtggaacttttaagtggagtggtggatcagaccaaagatggattaatatcttttcaagaatttgttgcctttgaatctgtcctgtgtgcccctgatgctttgtttatggtagcctttcagctgtttgacaaagctggcaaaggagaagtaacttttgaggatgttaagcaagtttttggacagaccacaattcatcaacatattccatttaactgggattcagaatttgtgcaactacattttggaaaagaaagaaaaagacacctgacatatgcggaatttactcagtttttattggaaatacaactggagcacgcaaagcaagcctttgtgcaacgggacaatgctaggactgggagagtcacagccatcgacttccgagacatcatggtcaccatccgcccccatgtcttgactccttttgtagaagaatgtctagtagctgctgctggaggtaccacatcccatcaagttagtttctcctattttaatggatttaattcgctccttaacaacatggaactcattagaaagatctatagcactctggctggcaccaggaaagatgttgaagtgactaaggaggagtttgttctggcagctcagaaatttggtcaggttacacccatggaagttgacatcttgtttcagttagcagatttatatgagccaaggggacgtatgaccttagcagacattgaacggattgctcctctggaagagggaactctgccctttaacttggctgaggcccagaggcagcagaaggcctcaggtgattcagctcgaccagttcttctacaagttgcagagtcggcctacaggtttggtctgggttctgttgctggagctgttggagccactgctgtgtatcctatcgatcttgtaaaaactcgaatgcagaaccaacgatcaactggctcttttgtgggagaactcatgtataaaaacagctttgactgttttaagaaagtgctacgctatgaaggcttctttggactgtatagaggtctgttgccacagttattgggagttgccccagagaaggccataaaacttacagtgaacgattttgtgagggataaatttatgcacaaagatggttcggtcccacttgcagcagaaattcttgctggaggctgcgctggaggctcccaggtgattttcacaaatcctttagaaatcgtcaagatccgtttgcaagtggcaggagaaatcaccactggtcctcgagtcagtgctctgtctgtcgtgcgggacctggggttttttgggatctacaagggtgccaaagcatgctttctgcgggacattcctttctcggccatctactttccgtgctatgctcatgtgaaggcttcctttgcaaatgaagatgggcaggttagcccaggaagcctgctcttagctggtgccatagctggtatgcctgcagcatctttagtgacccctgctgatgttatcaagacgagattacaggtggctgcccgggctggccaaaccacttacagcggagtgatagactgctttagaaagatactgcgtgaagaaggaccaaaagctctgtggaagggagctggtgctcgtgtatttcgatcctcaccccagtttggtgtaactttgctgacttacgaattgctacagcgatggttctacattgattttggaggagtaaaacccatgggatcagagccagttcctaaatccaggatcaacctgcctgccccgaatcctgatcacgttgggggctacaaactggcagttgctacatttgcagggattgaaaacaaatttggactttacctacctctcttcaagccatcagtatctacctcaaaggctattggtggaggcccataggaagatcagccctgggatagtgctgtctttttgtgggtactgcagtaaagaacatccctcctgggaatgaagcaatgcttcatcccttttacgtccatctcttgtttaaattcaagtccaggcttttttatcatgtgaaatcattcattttctgggtgttttcttaaccagatcattgtgaaattattcataattattatttggccctctgcccagaaacctttgtttgcatctgaaaattgatgggatttggtcaacactaacatgatttggggaaaggagcaagtcagaatagaaattagtactcccctccttgaactaggattgtagtcccaaagaggctactgtaaggcaatcatggtgctcagagcagtgtttcgtgtgtgttttaaactggtaggaaactaggtgcatatttataaaaataaaaaacactgggagaaatgaaaaaatatatatcaaatatattcagcctggcttcaaattgtaagcatgcacaaattctgtctctggattatattatgaagcttttatgtgaaacatgtttctttgtaatgaaaaccacattggagatgtttagtaatcatattgttactggtaccaagactactagggaaatgcctttgtactttagggaagtacttttggcattttactgtacagacagaaaaaactgagatgtagcccctctcctggaagtgctaattttgaaaaactgctcatatgatgtacatgtactgattactgcctattttaataaacactcttgaaaaatgcatgttgccctgttgctgcctgccctattctcctcatctccccatcattggtacccacttgcttttaaaatccactttatcttgaataatgtaagacaaatatgttctgacataagtatttaattcatgttgccttgcataatggtcagaggcgcatgaatttgtgaaggtggaaataaactatttgtaaagtgaaaaaaaaaaaaaaa

SEQ ID NO: 3 - protein, 675 aa, homo sapiens
MAAAKVALTKRADPAELRTIFLKYASIEKNGEFFMSPNDFVTRYLNIFGESQPNPKTVELLSGVVDQTKDGLISFQEFVAFESVLCAPDALFMVAFQLFDKAGKGEVTFEDVKQVFGQTTIHQHIPFNWDSEFVQLHFGKERKRHLTYAEFTQFLLEIQLEHAKQAFVQRDNARTGRVTAIDFRDIMVTIRPHVLTPFVEECLVAAAGGTTSHQVSFSYFNGFNSLLNNMELIRKIYSTLAGTRKDVEVTKEEFVLAAQKFGQVTPMEVDILFQLADLYEPRGRMTLADIERIAPLEEGTLPFNLAEAQRQKASGDSARPVLLQVAESAYRFGLGSVAGAVGATAVYPIDLVKTRMQNQRSTGSFVGELMYKNSFDCFKKVLRYEGFFGLYRGLLPQLLGVAPEKAIKLTVNDFVRDKFMHKDGSVPLAAEILAGGCAGGSQVIFTNPLEIVKIRLQVAGEITTGPRVSALSVVRDLGFFGIYKGAKACFLRDIPFSAIYFPCYAHVKASFANEDGQVSPGSLLLAGAIAGMPAASLVTPADVIKTRLQVAARAGQTTYSGVIDCFRKILREEGPKALWKGAGARVFRSSPQFGVTLLTYELLQRWFYIDFGGVKPMGSEPVPKSRINLPAPNPDHVGGYKLAVATFAGIENKFGLYLPLFKPSVSTSKAIGGGP

SEQ ID NO: 4 - DNA, 3204 bp, homo sapiens
aatgggcgggcagcatccacatgacccgcgccggcgggagggcgtggggaggcaggccaggaacgcacgctgcctggccgtatcgccgcccccaccgccgccgccgccgggactagaagtgagccgcccgggtcccaaacgccagccagccagtcagtgggtcccgcagtcgcccgcaaccggggcgaatcatggcggccgccaaggtggctttaaccaagagagcagatccagctgagcttagaacaatatttttgaagtatgcaagcattgagaaaaacggtgaatttttcatgtcccccaatgactttgtcactcgatacttgaacatttttggagaaagccagcctaatccaaagactgtggaacttttaagtggagtggtggatcagaccaaagatggattaatatcttttcaagaatttgttgcctttgaatctgtcctgtgtgcccctgatgctttgtttatggtagcctttcagctgtttgacaaagctggcaaaggagaagtaacttttgaggatgttaagcaagtttttggacagaccacaattcatcaacatattccatttaactgggattcagaatttgtgcaactacattttggaaaagaaagaaaaagacacctgacatatgcggaatttactcagtttttattggaaatacaactggagcacgcaaagcaagcctttgtgcaacgggacaatgctaggactgggagagtcacagccatcgacttccgagacatcatggtcaccatccgcccccatgtcttgactccttttgtagaagaatgtctagtagctgctgctggaggtaccacatcccatcaagttagtttctcctattttaatggatttaattcgctccttaacaacatggaactcattagaaagatctatagcactctggctggcaccaggaaagatgttgaagtgactaaggaggagtttgttctggcagctcagaaatttggtcaggttacacccatggaagttgacatcttgtttcagttagcagatttatatgagccaaggggacgtatgaccttagcagacattgaacggattgctcctctggaagagggaactctgccctttaacttggctgaggcccagaggcagaaggcctcaggtgattcagctcgaccagttcttctacaagttgcagagtcggcctacaggtttggtctgggttctgttgctggagctgttggagccactgctgtgtatcctatcgatcttgtaaaaactcgaatgcagaaccaacgatcaactggctcttttgtgggagaactcatgtataaaaacagctttgactgttttaagaaagtgctacgctatgaaggcttctttggactgtatagaggtctgttgccacagttattgggagttgccccagagaaggccataaaacttacagtgaacgattttgtgagggataaatttatgcacaaagatggttcggtcccacttgcagcagaaattcttgctggaggctgcgctggaggctcccaggtgattttcacaaatcctttagaaatcgtcaagatccgtttgcaagtggcaggagaaatcaccactggtcctcgagtcagtgctctgtctgtcgtgcgggacctggggttttttgggatctacaagggtgccaaagcatgctttctgcgggacattcctttctcggccatctactttccgtgctatgctcatgtgaaggcttcctttgcaaatgaagatgggcaggttagcccaggaagcctgctcttagctggtgccatagctggtatgcctgcagcatctttagtgacccctgctgatgttatcaagacgagattacaggtggctgcccgggctggccaaaccacttacagcggagtgatagactgctttagaaagatactgcgtgaagaaggaccaaaagctctgtggaagggagctggtgctcgtgtatttcgatcctcaccccagtttggtgtaactttgctgacttacgaattgctacagcgatggttctacattgattttggaggagtaaaacccatgggatcagagccagttcctaaatccaggatcaacctgcctgccccgaatcctgatcacgttgggggctacaaactggcagttgctacatttgcagggattgaaaacaaatttggactttacctacctctcttcaagccatcagtatctacctcaaaggctattggtggaggcccataggaagatcagccctgggatagtgctgtctttttgtgggtactgcagtaaagaacatccctcctgggaatgaagcaatgcttcatcccttttacgtccatctcttgtttaaattcaagtccaggcttttttatcatgtgaaatcattcattttctgggtgttttcttaaccagatcattgtgaaattattcataattattatttggccctctgcccagaaacctttgtttgcatctgaaaattgatgggatttggtcaacactaacatgatttggggaaaggagcaagtcagaatagaaattagtactcccctccttgaactaggattgtagtcccaaagaggctactgtaaggcaatcatggtgctcagagcagtgtttcgtgtgtgttttaaactggtaggaaactaggtgcatatttataaaaataaaaaacactgggagaaatgaaaaaatatatatcaaatatattcagcctggcttcaaattgtaagcatgcacaaattctgtctctggattatattatgaagcttttatgtgaaacatgtttctttgtaatgaaaaccacattggagatgtttagtaatcatattgttactggtaccaagactactagggaaatgcctttgtactttagggaagtacttttggcattttactgtacagacagaaaaaactgagatgtagcccctctcctggaagtgctaattttgaaaaactgctcatatgatgtacatgtactgattactgcctattttaataaacactcttgaaaaatgcatgttgccctgttgctgcctgccctattctcctcatctccccatcattggtacccacttgcttttaaaatccactttatcttgaataatgtaagacaaatatgttctgacataagtatttaattcatgttgccttgcataatggtcagaggcgcatgaatttgtgaaggtggaaataaactatttgtaaagtgaaaaaaaaaaaaaaa

SEQ ID NO: 5 - DNA, 21 bp, Artificial sequence
  Human ASS1-forward primer
ttataacctgggatgggcacc

SEQ ID NO: 6 - DNA, 21 bp, Artificial sequence
  Human ASS1-reverse primer
tggacatagcgtctgggattg

SEQ ID NO: 7 - DNA, 20 bp, Artificial sequence
  Human ASL-forward primer
tgatgccccagaagaaaaac

SEQ ID NO: 8 - DNA, 20 bp, Artificial sequence
  Human ASL-reverse primer
ttggtgaatctgcagcgtag

SEQ ID NO: 9 - DNA, 23 bp, Artificial sequence
  Human ARG1-forward primer
gggaacactacattttgaaaact

SEQ ID NO: 10 - DNA, 23 bp, Artificial sequence
  Human ARG1-reverse primer
ggccttttctttcttcctagtag

SEQ ID NO: 11 - DNA, 20 bp, Artificial sequence
  Human ARG2-forward primer
accctcctgaacattttatt

SEQ ID NO: 12 - DNA, 20 bp, Artificial sequence
  Human ARG2- reverse primer
ggtctttgtctcttgccaat

SEQ ID NO: 13 - DNA, 20 bp, Artificial sequence
  Human CPS1-forward primer
ggcaatggctacaggaagaa

SEQ ID NO: 14 - DNA, 21 bp, Artificial sequence
  Human CPS1-reverse primer
cccaagcatggtacccttatc

SEQ ID NO: 15 - DNA, 22 bp, Artificial sequence
  Human CPS2-forward primer
ctgaagggatggaaggagattg

SEQ ID NO: 16 - DNA, 20 bp, Artificial sequence
  Human CPS2-reverse primer
gggtccaagttctccatgtt

SEQ ID NO: 17 - DNA, 22 bp, Artificial sequence
  Human HPRT-forward primer
attgacactggcaaaacaatgc

SEQ ID NO: 18 - DNA, 20 bp, Artificial sequence
  Human HPRT-reverse primer
tccaacacttcgtggggtcc

SEQ ID NO: 19 - DNA, 21 bp, Artificial sequence
  ASS1 shRNA
aatgagcatggtaaaggatgg

SEQ ID NO: 20 - DNA, 21 bp, Artificial sequence
  ASS1 shRNA
aaccggttgtagaattcaggc

SEQ ID NO: 21 - DNA, 1476 bp, Artificial sequence
  Ass1 probe
gcgcatccagggttataagcgcggagcagggctgagagaggcccaggcggcagtggcagaggccagatgaaccactcagtagtccgtgtagtctgctttcgattataggtacaggtccctccttgctggacatcttgtctctgtaggccagaaccacagagcaggaggtgtccaggccacgttccttcagccacacgaggtaggcgatgacatcatagcccttctggccaatgttggccatcctggcttcctcaaagtctgccccaagcttcagcgccttatcctcaatgaacaccttttcttccacaaattccttgctctggacagcaggccagatgaagtcctcgtagagtgcactgggagaggtgcccaggagatagcgagctatgcaaggcctggcctgggcaatctccacctgtcacacatacttggccccttcaccctttcccgtggcgccgtgctcaaagcggacctggtcatgtgccagtgaatagcaggtgggagcgatgaccttaatctgtgtaaaactcaggcatcctcatcatttcggcccttgaaccgttgctttgcatactccatcgtgacagggatggggattcccatactccaggggctcttggtgatgtgcatgaggttttcatccaggatcccagcctcataaggtgcttgattcttggggtgagtttttgtgtagagacccttgggtgctttggcagggtctatctcaaggacatctgggccagggaccccttttttgaattctttgatgttggtcaccttggatgtggtgcgggttgtgctcaggtacatgaagagttccccgtgcttgcccgcaacttccacgatgtcaatgcgacccatcattccaatgaagcggttcgtctcgtagatacctcgggagtaaaggatggtccctgctgcctctatgtctaaatgagcgacttcccgatccatcgtgaacaggccctgcttgattcgagctctgcgaatttgaggctgtgccagaaacctgtgtagcgaacaaattcacattcagcctgggacttctggatacagtgcaccttcccttctacccgttggcccttgaagacagacaactcccgaccgaggatgtactcattgtagagtgaaagtggtgcacgttcatgctcaccaggtcgatgggctcatagtcgctgatattgatgaagccagtgtactccttcagcctgagcgagaccttgctctgaaggcgatttgtcagggtctatttggcagagtggggaggcccgctcctgctgaagcctgggagagctgcaaatttatcacaacaattaggtggagaacaagctacaatgacacagcagccccagtcagaggctgtgggggggcggggggctataggggaccagggaacccttggatgaccacttttgtagctgcccgccaccctccctattgtcattttatgctttctaagactaatgtaacttcttt

SEQ ID NO: 22 - DNA, 20 bp, Artificial sequence
  Human Citrin forward primer
tgagccaaggggacgtatga

SEQ ID NO: 23 - DNA, 20 bp, Artificial sequence
  Human Citrin reverse primer
tgagccaaggggacgtatga

SEQ ID NO: 24 - DNA, 22 bp, Artificial sequence
  Human HPRT forward primer
attgacactggcaaaacaatgc

SEQ ID NO: 25 - DNA, 20 bp, Artificial sequence
  Human HPRT reverse primer
tccaacacttcgtggggtcc

SEQ ID NO: 26 - DNA, 19 bp, Artificial sequence
  Citrin silencing, sh901
ttaagaaagtgctacgcta

SEQ ID NO: 27 - DNA, 19 bp, Artificial sequence
  Citrin silencing, sh909
gtatcctatcgatcttgta

SEQ ID NO: 28 - protein, 2225 aa, homo sapiens
MAALVLEDGSVLRGQPFGAAVSTAGEVVFQTGMVGYPEALTDPSYKAQILVLTYPLIGNYGIPPDEMDEFGLCKWFESSGIHVAALVVGECCPTPSHWSATRTLHEWLQQHGIPGLQGVDTRELTKKLREQGSLLGKLVQNGTEPSSLPFLDPNARPLVPEVSIKTPRVFNTGGAPRILALDCGLKYNQIRCLCQRGAEVTVVPWDHALDSQEYEGLFLSNGPGDPASYPSVVSTLSRVLSEPNPRPVFGICLGHQLLALAIGAKTYKMRYGNRGHNQPCLLVGSGRCFLTSQNHGFAVETDSLPADWAPLFTNANDGSNEGIVHNSLPFFSVQFHPEHQAGPSDMELLFDIFLETVKEATAGNPGGQTVRERLTERLCPPGIPTPGSGLPPPRKVLILGSGGLSIGQAGEFDYSGSQAIKALKEENIQTLLINPNIATVQTSQGLADKVYFLPITPHYVTQVIRNERPDGVLLTFGGQTALNCGVELTKAGVLARYGVRVLGTPVETIELTEDRRAFAARMAEIGEHVAPSEAANSLEQAQAAAERLGYPVLVRAAFALGGLGSGFASNREELSALVAPAFAHTSQVLVDKSLKGWKEIEYEVVRDAYGNCVTVCNMENLDPLGIHTGESIVVAPSQTLNDREYQLLRQTAIKVTQHLGIVGECNVQYALNPESEQYYIIEVNARLSRSSALASKATGYPLAYVAAKLALGIPLPELRNSVTGGTAAFEPSVDYCVVKIPRWDLSKFLRVSTKIGSCMKSVGEVMGIGRSFEEAFQKALRMVDENCVGFDHTVKPVSDMELETPTDKRIFVVAAALWAGYSVDRLYELTRIDRWFLHRMKRIIAHAQLLEQHRGQPLPPDLLQQAKCLGFSDKQIALAVLSTELAVRKLRQELGICPAVKQIDTVAAEWPAQTNYLYLTYWGTTHDLTFRTPHVLVLGSGVYRIGSSVEFDWCAVGCIQQLRKMGYKTIMVNYNPETVSTDYDMCDRLYFDEISFEVVMDIYELENPEGVILSMGGQLPNNMAMALHRQQCRVLGTSPEAIDSAENRFKFSRLLDTIGISQPQWRELSDLESARQFCQTVGYPCVVRPSYVLSGAAMNVAYTDGDLERFLSSAAAVSKEHPVVISKFIQEAKEIDVDAVASDGVVAAIAISEHVENAGVHSGDATLVTPPQDITAKTLERIKAIVHAVGQELQVTGPFNLQLIAKDDQLKVIECNVRVSRSFPFVSKTLGVDLVALATRVIMGEEVEPVGLMTGSGVVGVKVPQFSFSRLAGADVVLGVEMTSTGEVAGFGESRCEAYLKAMLSTGFKIPKKNILLTIGSYKNKSELLPTVRLLESLGYSLYASLGTADFYTEHGVKVTAVDWHFEEAVDGECPPQRSILEQLAEKNFELVINLSMRGAGGRRLSSFVTKGYRTRRLAADFSVPLIIDIKCTKLFVEALGQIGPAPPLKVHVDCMTSQKLVRLPGLIDVHVHLREPGGTHKEDFASGTAAALAGGITMVCAMPNTRPPIIDAPALALAQKLAEAGARCDFALFLGASSENAGTLGTVAGSAAGLKLYLNETFSELRLDSVVQWMEHFETWPSHLPIVAHAEQQTVAAVLMVAQLTQRSVHICHVARKEEILLIKAAKARGLPVTCEVAPHHLFLSHDDLERLGPGKGEVRPELGSRQDVEALWENMAVIDCFASDHAPHTLEEKCGSRPPPGFPGLETMLPLLLTAVSEGRLSLDDLLQRLHHNPRRIFHLPPQEDTYVEVDLEHEWTIPSHMPFSKAHWTPFEGQKVKGTVRRVVLRGEVAYIDGQVLVPPGYGQDVRKWPQGAVPQLPPSAPATSEMTTTPERPRRGIPGLPDGRFHLPPRIHRASDPGLPAEEPKEKSSRKVAEPELMGTPDGTCYPPPPVPRQASPQNLGTPGLLHPQTSPLLHSLVGQHILSVQQFTKDQMSHLFNVAHTLRMMVQKERSLDILKGKVMASMFYEVSTRTSSSFAAAMARLGGAVLSFSEATSSVQKGESLADSVQTMSCYADVVVLRHPQPGAVELAAKHCRRPVINAGDGVGEHPTQALLDIFTIREELGTVNGMTITMVGDLKHGRTVHSLACLLTQYRVSLRYVAPPSLRMPPTVRAFVASRGTKQEEFESIEEALPDTDVLYMTRIQKERFGSTQEYEACFGQFILTPHIMTRAKKKMVVMHPMPRVNEISVEVDSDPRAAYFRQAENGMYIRMALLATVLGRF

SEQ ID NO: 29 - DNA, 7131 bp, homo sapiens
gcgcgcccgaggctcctacgctgccgcgcccggcttctctccagcgccccgcgccgttagccacgtggaccgactccggcgcgccgtcctcacgtggttccagtggagtttgcagtccttcccgcttctccgtactcgcccccgcctctgagctcccttcccatggcggccctagtgttggaggacgggtcggtcctgcggggccagccctttggggccgccgtgtcgactgccggggaagtggtgtttcaaaccggcatggtcggctaccccgaggccctcactgatccctcctacaaggcacagatcttagtgctcacctatcctctgatcggcaactatggcatccccccagatgaaatggatgagttcggtctctgcaagtggtttgaatcctcgggcatccacgtagcagcactggtagtgggagagtgctgtcctactcccagccactggagtgccacccgcaccctgcatgagtggctgcagcagcatggcatccctggcttgcaaggagtagacactcgggagctgaccaagaagttgcgggaacaggggtctctgctggggaagctggtccagaatggaacagaaccttcatccctgccattcttggaccccaatgcccgccccctggtaccagaggtctccattaagactccacgggtattcaatacagggggtgcccctcggatccttgctttggactgtggcctcaagtataatcagatccgatgcctctgccagcgtggggctgaggtcactgtggtaccctgggaccatgcactagacagccaagagtatgagggtctcttcttaagtaatgggcctggtgaccctgcctcctatcccagtgtcgtatccacactgagccgtgttttatctgagcctaatccccgacctgtctttgggatctgcctgggacaccagctattggccttagccattggggccaagacttacaagatgagatatgggaaccgaggccataaccagccctgcttgttggtgggctctgggcgctgctttctgacatcccagaaccatgggtttgctgtggagacagactcactgccagcagactgggctcctctcttcaccaacgccaatgatggttccaatgaaggcattgtgcacaacagcttgcctttcttcagtgtccagtttcacccagagcaccaagctggcccttcagatatggaactgcttttcgatatctttctggaaactgtgaaagaggccacagctgggaaccctgggggccagacagttagagagcggctgactgagcgcctctgtccccctgggattcccactcccggctctggacttccaccaccacgaaaggttctgatcctgggctcagggggcctctccattggccaagctggagaatttgactactcgggctctcaggcaattaaggccctgaaggaggaaaacatccagacgttgctgatcaaccccaatattgccacagtgcagacctcccaggggctggccgacaaggtctattttcttcccataacacctcattatgtaacccaggtgatacgtaatgaacgccccgatggtgtgttactgacttttgggggccagactgctctgaactgtggtgtggagctgaccaaggccggggtgctggctcggtatggggtccgggtcctgggcacaccagtggagaccattgagctgaccgaggatcgacgggcctttgctgccagaatggcagagatcggagagcatgtggccccgagcgaggcagcaaattctcttgaacaggcccaggcagccgctgaacggctggggtaccctgtgctagtgcgtgcagcctttgccctgggtggcctgggctctggctttgcctctaacagggaggagctctctgctctcgtggccccagcttttgcccataccagccaagtgctagtagacaagtctctgaagggatggaaggagattgagtacgaggtggtgagagacgcctatggcaactgtgtcacggtgtgtaacatggagaacttggacccactgggcatccacactggtgagtccatagtggtggcccctagccagacactgaatgacagggagtatcagctcctgaggcagacagctatcaaggtgacccagcacctgggaattgttggggagtgcaatgtgcagtatgccttgaaccctgagtctgagcagtattacatcattgaagtgaatgccaggctctctcgcagctctgccctggccagtaaggccacaggttatccactggcttatgtggcagccaagctagcattgggcatccctttgcctgagctcaggaactctgtgacagggggtacagcagcctttgaacccagcgtggattattgtgtggtgaagattcctcgatgggaccttagcaagttcctgcgagtcagcacaaagattgggagctgcatgaagagcgttggtgaagtcatgggcattgggcgttcatttgaggaggccttccagaaggccctgcgcatggtggatgagaactgtgtgggctttgatcacacagtgaaaccagtcagcgatatggagttggagactccaacagataagcggatttttgtggtggcagctgctttgtgggctggttattcagtggaccgcctgtatgagctcacacgcatcgaccgctggttcctgcaccgaatgaagcgtatcatcgcacatgcccagctgctagaacaacaccgtggacagcctttgccgccagacctgctgcaacaggccaagtgtcttggcttctcagacaaacagattgcccttgcagttctgagcacagagctggctgttcgcaagctgcgtcaggaactggggatctgtccagcagtgaaacagattgacacagttgcagctgagtggccagcccagacaaattacctatacctaacgtattggggcaccacccatgacctcacctttcgaacacctcatgtcctagtccttggctctggcgtctaccgtattggctctagcgttgaatttgactggtgtgctgtaggctgcatccagcagctccgaaagatgggatataagaccatcatggtgaactataacccagagacagtcagcaccgactatgacatgtgtgatcgactctactttgatgagatctcttttgaggtggtgatggacatctatgagctcgagaaccctgaaggtgtgatcctatccatgggtggacagctgcccaacaacatggccatggcgttgcatcggcagcagtgccgggtgctgggcacctcccctgaagccattgactcggctgagaaccgtttcaagttttcccggctccttgacaccattggtatcagccagcctcagtggagggagctcagtgacctcgagtctgctcgccaattctgccagaccgtggggtacccctgtgtggtgcgcccctcctatgtgctgagcggtgctgctatgaatgtggcctacacggatggagacctggagcgcttcctgagcagcgcagcagccgtctccaaagagcatcccgtggtcatctccaagttcatccaggaggctaaggagattgacgtggatgccgtggcctctgatggtgtggtggcagccatcgccatctctgagcatgtggagaatgcaggtgtgcattcaggtgatgcgacgctggtgacccccccacaagatatcactgccaaaaccctggagcggatcaaagccattgtgcatgctgtgggccaggagctacaggtcacaggacccttcaatctgcagctcattgccaaggatgaccagctgaaagttattgaatgcaacgtacgtgtctctcgctccttccccttcgtttccaagacactgggtgtggacctagtagccttggccacgcgggtcatcatgggggaagaagtggaacctgtggggctaatgactggttctggagtcgtgggagtaaaggtgcctcagttctccttctcccgcttggcgggtgctgacgtggtgttgggtgtggaaatgaccagtactggggaggtggccggctttggggagagccgctgtgaggcatacctcaaggccatgctaagcactggctttaagatccccaagaagaatatcctgctgaccattggcagctataagaacaaaagcgagctgctcccaactgtgcggctactggagagcctgggctacagcctctatgccagtctcggcacagctgacttctacactgagcatggcgtcaaggtaacagctgtggactggcactttgaggaggctgtggatggtgagtgcccaccacagcggagcatcctggagcagctagctgagaaaaactttgagctggtgattaacctgtcaatgcgtggagctgggggccggcgtctctcttcctttgtcaccaagggctaccgcacccgacgcttggccgctgacttctccgtgcccctaatcatcgatatcaagtgcaccaaactctttgtggaggccctaggccagatcgggccagcccctcctttgaaggtgcatgttgactgtatgacctcccaaaagcttgtgcgactgccgggattgattgatgtccatgtgcacctgcgggaaccaggtgggacacataaggaggactttgcttcaggcacagccgctgccctggctgggggtatcaccatggtgtgtgccatgcctaatacccggccccccatcattgacgcccctgctctggccctggcccagaagctggcagaggctggcgcccggtgcgactttgcgctattccttggggcctcgtctgaaaatgcaggaaccttgggcaccgtggccgggtctgcagccgggctgaagctttacctcaatgagaccttctctgagctgcggctggacagcgtggtccagtggatggagcatttcgagacatggccctcccacctccccattgtggctcacgcagagcagcaaaccgtggctgctgtcctcatggtggctcagctcactcagcgctcagtgcacatatgtcacgtggcacggaaggaggagatcctgctaattaaagctgcaaaggcacggggcttgccagtgacctgcgaggtggctccccaccacctgttcctaagccatgatgacctggagcgcctggggcctgggaagggggaggtccggcctgagcttggctcccgccaggatgtggaagccctgtgggagaacatggctgtcatcgactgctttgcctcagaccatgctccccataccttggaggagaagtgtgggtccaggcccccacctgggttcccagggttagagaccatgctgccactactcctgacggctgtaagcgagggccggctcagcctggacgacctgctgcagcgattgcaccacaatcctcggcgcatctttcacctgcccccgcaggaggacacctatgtggaggtggatctggagcatgagtggacaattcccagccacatgcccttctccaaggcccactggacaccttttgaagggcagaaagtgaagggcaccgtccgccgtgtggtcctgcgaggggaggttgcctatatcgatgggcaggttctggtacccccgggctatggacaggatgtacggaagtggccacagggggctgttcctcagctcccaccctcagcccctgccactagtgagatgaccacgacacctgaaagaccccgccgtggcatcccagggcttcctgatggccgcttccatctgccgccccgaatccatcgagcctccgacccaggtttgccagctgaggagccaaaggagaagtcctctcggaaggtagccgagccagagctgatgggaacccctgatggcacctgctaccctccaccaccagtaccgagacaggcatctccccagaacctggggacccctggcttgctgcacccccagacctcacccctgctgcactcattagtgggccaacatatcctgtccgtccagcagttcaccaaggatcagatgtctcacctgttcaatgtggcacacacactgcgtatgatggtgcagaaggagcggagcctcgacatcctgaaggggaaggtcatggcctccatgttctatgaagtgagcacacggaccagcagctcctttgcagcagccatggcccggctgggaggtgctgtgctcagcttctcggaagccacatcgtccgtccagaagggcgaatccctggctgactccgtgcagaccatgagctgctatgccgacgtcgtcgtgctccggcacccccagcctggagcagtggagctggccgccaagcactgccggaggccagtgatcaatgctggggatggggtcggagagcaccccacccaggccctgctggacatcttcaccatccgtgaggagctgggaactgtcaatggcatgacgatcacgatggtgggtgacctgaagcacggacgcacagtacattccctggcctgcctgctcacccagtatcgtgtcagcctgcgctacgtggcacctcccagcctgcgcatgccacccactgtgcgggccttcgtggcctcccgcggcaccaagcaggaggaattcgagagcattgaggaggcgctgcctgacactgatgtgctctacatgactcgaatccagaaggaacgatttggctctacccaggagtacgaagcttgctttggtcagttcatcctcactccccacatcatgacccgggccaagaagaagatggtggtgatgcacccgatgccccgtgtcaacgagataagcgtggaagtggactcggatccccgcgcagcctacttccgccaggctgagaacggcatgtacatccgcatggctctgttagccaccgtgctgggccgtttctagggcctggcttcctcagcctcttctctttaggcccagctgctgggcaaggaattccagtgcctcctacgggggcagcacacttagatattcctggacatccagatagctcacatgtgctgaccacacttcaggctctggactggagctctctggcatgggggtggggcctcagatgctggggcccagtctgccccatcttcattcctgcaccttaaacctgtacagtcatttttctactgacttaataaacagccgagctgtcccttgatgctgaaaaaaaaaaaaaaaaaa

SEQ ID NO: 30 - protein, 2162 aa, homo sapiens
MAALVLEDGSVLRGQPFGAAVSTAGEVVFQTGMVGYPEALTDPSYKAQILVLTYPLIGNYGIPPDEMDEFGLCKWFESSGIHVAALVVGECCPTPSHWSATRTLHEWLQQHGIPGLQGVDTRELTKKLREQGSLLGKLVQNGTEPSSLPFLDPNARPLVPEVSIKTPRVFNTGGAPRILALDCGLKYNQIRCLCQRGAEVTVVPWDHALDSQEYEGLFLSNGPGDPASYPSVVSTLSRVLSEPNPRPVFGICLGHQLLALAIGAKTYKMRYGNRGHNQPCLLVGSGRCFLTSQNHGFAVETDSLPADWAPLFTNANDGSNEGIVHNSLPFFSVQFHPEHQAGPSDMELLFDIFLETVKEATAGNPGGQTVRERLTERLCPPGIPTPGSGLPPPRKVLILGSGGLSIGQAGEFDYSGSQAIKALKEENIQTLLINPNIATVQTSQGLADKVYFLPITPHYVTQVIRNERPDGVLLTFGGQTALNCGVELTKAGVLARYGVRVLGTPVETIELTEDRRAFAARMAEIGEHVAPSEAANSLEQAQAAAERLGYPVLVRAAFALGGLGSGFASNREELSALVAPAFAHTSQVLVDKSLKGWKEIEYEVVRDAYGNCVTYYIIEVNARLSRSSALASKATGYPLAYVAAKLALGIPLPELRNSVTGGTAAFEPSVDYCVVKIPRWDLSKFLRVSTKIGSCMKSVGEVMGIGRSFEEAFQKALRMVDENCVGFDHTVKPVSDMELETPTDKRIFVVAAALWAGYSVDRLYELTRIDRWFLHRMKRIIAHAQLLEQHRGQPLPPDLLQQAKCLGFSDKQIALAVLSTELAVRKLRQELGICPAVKQIDTVAAEWPAQTNYLYLTYWGTTHDLTFRTPHVLVLGSGVYRIGSSVEFDWCAVGCIQQLRKMGYKTIMVNYNPETVSTDYDMCDRLYFDEISFEVVMDIYELENPEGVILSMGGQLPNNMAMALHRQQCRVLGTSPEAIDSAENRFKFSRLLDTIGISQPQWRELSDLESARQFCQTVGYPCVVRPSYVLSGAAMNVAYTDGDLERFLSSAAAVSKEHPVVISKFIQEAKEIDVDAVASDGVVAAIAISEHVENAGVHSGDATLVTPPQDITAKTLERIKAIVHAVGQELQVTGPFNLQLIAKDDQLKVIECNVRVSRSFPFVSKTLGVDLVALATRVIMGEEVEPVGLMTGSGVVGVKVPQFSFSRLAGADVVLGVEMTSTGEVAGFGESRCEAYLKAMLSTGFKIPKKNILLTIGSYKNKSELLPTVRLLESLGYSLYASLGTADFYTEHGVKVTAVDWHFEEAVDGECPPQRSILEQLAEKNFELVINLSMRGAGGRRLSSFVTKGYRTRRLAADFSVPLIIDIKCTKLFVEALGQIGPAPPLKVHVDCMTSQKLVRLPGLIDVHVHLREPGGTHKEDFASGTAAALAGGITMVCAMPNTRPPIIDAPALALAQKLAEAGARCDFALFLGASSENAGTLGTVAGSAAGLKLYLNETFSELRLDSVVQWMEHFETWPSHLPIVAHAEQQTVAAVLMVAQLTQRSVHICHVARKEEILLIKAAKARGLPVTCEVAPHHLFLSHDDLERLGPGKGEVRPELGSRQDVEALWENMAVIDCFASDHAPHTLEEKCGSRPPPGFPGLETMLPLLLTAVSEGRLSLDDLLQRLHHNPRRIFHLPPQEDTYVEVDLEHEWTIPSHMPFSKAHWTPFEGQKVKGTVRRVVLRGEVAYIDGQVLVPPGYGQDVRKWPQGAVPQLPPSAPATSEMTTTPERPRRGIPGLPDGRFHLPPRIHRASDPGLPAEEPKEKSSRKVAEPELMGTPDGTCYPPPPVPRQASPQNLGTPGLLHPQTSPLLHSLVGQHILSVQQFTKDQMSHLFNVAHTLRMMVQKERSLDILKGKVMASMFYEVSTRTSSSFAAAMARLGGAVLSFSEATSSVQKGESLADSVQTMSCYADVVVLRHPQPGAVELAAKHCRRPVINAGDGVGEHPTQALLDIFTIREELGTVNGMTITMVGDLKHGRTVHSLACLLTQYRVSLRYVAPPSLRMPPTVRAFVASRGTKQEEFESIEEALPDTDVLYMTRIQKERFGSTQEYEACFGQFILTPHIMTRAKKKMVVMHPMPRVNEISVEVDSDPRAAYFRQAENGMYIRMALLATVLGRF

SEQ ID NO: 31 - DNA, 6942 bp, homo sapiens
gcgcgcccgaggctcctacgctgccgcgcccggcttctctccagcgccccgcgccgttagccacgtggaccgactccggcgcgccgtcctcacgtggttccagtggagtttgcagtccttcccgcttctccgtactcgcccccgcctctgagctcccttcccatggcggccctagtgttggaggacgggtcggtcctgcggggccagccctttggggccgccgtgtcgactgccggggaagtggtgtttcaaaccggcatggtcggctaccccgaggccctcactgatccctcctacaaggcacagatcttagtgctcacctatcctctgatcggcaactatggcatccccccagatgaaatggatgagttcggtctctgcaagtggtttgaatcctcgggcatccacgtagcagcactggtagtgggagagtgctgtcctactcccagccactggagtgccacccgcaccctgcatgagtggctgcagcagcatggcatccctggcttgcaaggagtagacactcgggagctgaccaagaagttgcgggaacaggggtctctgctggggaagctggtccagaatggaacagaaccttcatccctgccattcttggaccccaatgcccgccccctggtaccagaggtctccattaagactccacgggtattcaatacagggggtgcccctcggatccttgctttggactgtggcctcaagtataatcagatccgatgcctctgccagcgtggggctgaggtcactgtggtaccctgggaccatgcactagacagccaagagtatgagggtctcttcttaagtaatgggcctggtgaccctgcctcctatcccagtgtcgtatccacactgagccgtgttttatctgagcctaatccccgacctgtctttgggatctgcctgggacaccagctattggccttagccattggggccaagacttacaagatgagatatgggaaccgaggccataaccagccctgcttgttggtgggctctgggcgctgctttctgacatcccagaaccatgggtttgctgtggagacagactcactgccagcagactgggctcctctcttcaccaacgccaatgatggttccaatgaaggcattgtgcacaacagcttgcctttcttcagtgtccagtttcacccagagcaccaagctggcccttcagatatggaactgcttttcgatatctttctggaaactgtgaaagaggccacagctgggaaccctgggggccagacagttagagagcggctgactgagcgcctctgtccccctgggattcccactcccggctctggacttccaccaccacgaaaggttctgatcctgggctcagggggcctctccattggccaagctggagaatttgactactcgggctctcaggcaattaaggccctgaaggaggaaaacatccagacgttgctgatcaaccccaatattgccacagtgcagacctcccaggggctggccgacaaggtctattttcttcccataacacctcattatgtaacccaggtgatacgtaatgaacgccccgatggtgtgttactgacttttgggggccagactgctctgaactgtggtgtggagctgaccaaggccggggtgctggctcggtatggggtccgggtcctgggcacaccagtggagaccattgagctgaccgaggatcgacgggcctttgctgccagaatggcagagatcggagagcatgtggccccgagcgaggcagcaaattctcttgaacaggcccaggcagccgctgaacggctggggtaccctgtgctagtgcgtgcagcctttgccctgggtggcctgggctctggctttgcctctaacagggaggagctctctgctctcgtggccccagcttttgcccataccagccaagtgctagtagacaagtctctgaagggatggaaggagattgagtacgaggtggtgagagacgcctatggcaactgtgtcacgtattacatcattgaagtgaatgccaggctctctcgcagctctgccctggccagtaaggccacaggttatccactggcttatgtggcagccaagctagcattgggcatccctttgcctgagctcaggaactctgtgacagggggtacagcagcctttgaacccagcgtggattattgtgtggtgaagattcctcgatgggaccttagcaagttcctgcgagtcagcacaaagattgggagctgcatgaagagcgttggtgaagtcatgggcattgggcgttcatttgaggaggccttccagaaggccctgcgcatggtggatgagaactgtgtgggctttgatcacacagtgaaaccagtcagcgatatggagttggagactccaacagataagcggatttttgtggtggcagctgctttgtgggctggttattcagtggaccgcctgtatgagctcacacgcatcgaccgctggttcctgcaccgaatgaagcgtatcatcgcacatgcccagctgctagaacaacaccgtggacagcctttgccgccagacctgctgcaacaggccaagtgtcttggcttctcagacaaacagattgcccttgcagttctgagcacagagctggctgttcgcaagctgcgtcaggaactggggatctgtccagcagtgaaacagattgacacagttgcagctgagtggccagcccagacaaattacctatacctaacgtattggggcaccacccatgacctcacctttcgaacacctcatgtcctagtccttggctctggcgtctaccgtattggctctagcgttgaatttgactggtgtgctgtaggctgcatccagcagctccgaaagatgggatataagaccatcatggtgaactataacccagagacagtcagcaccgactatgacatgtgtgatcgactctactttgatgagatctcttttgaggtggtgatggacatctatgagctcgagaaccctgaaggtgtgatcctatccatgggtggacagctgcccaacaacatggccatggcgttgcatcggcagcagtgccgggtgctgggcacctcccctgaagccattgactcggctgagaaccgtttcaagttttcccggctccttgacaccattggtatcagccagcctcagtggagggagctcagtgacctcgagtctgctcgccaattctgccagaccgtggggtacccctgtgtggtgcgcccctcctatgtgctgagcggtgctgctatgaatgtggcctacacggatggagacctggagcgcttcctgagcagcgcagcagccgtctccaaagagcatcccgtggtcatctccaagttcatccaggaggctaaggagattgacgtggatgccgtggcctctgatggtgtggtggcagccatcgccatctctgagcatgtggagaatgcaggtgtgcattcaggtgatgcgacgctggtgacccccccacaagatatcactgccaaaaccctggagcggatcaaagccattgtgcatgctgtgggccaggagctacaggtcacaggacccttcaatctgcagctcattgccaaggatgaccagctgaaagttattgaatgcaacgtacgtgtctctcgctccttccccttcgtttccaagacactgggtgtggacctagtagccttggccacgcgggtcatcatgggggaagaagtggaacctgtggggctaatgactggttctggagtcgtgggagtaaaggtgcctcagttctccttctcccgcttggcgggtgctgacgtggtgttgggtgtggaaatgaccagtactggggaggtggccggctttggggagagccgctgtgaggcatacctcaaggccatgctaagcactggctttaagatccccaagaagaatatcctgctgaccattggcagctataagaacaaaagcgagctgctcccaactgtgcggctactggagagcctgggctacagcctctatgccagtctcggcacagctgacttctacactgagcatggcgtcaaggtaacagctgtggactggcactttgaggaggctgtggatggtgagtgcccaccacagcggagcatcctggagcagctagctgagaaaaactttgagctggtgattaacctgtcaatgcgtggagctgggggccggcgtctctcttcctttgtcaccaagggctaccgcacccgacgcttggccgctgacttctccgtgcccctaatcatcgatatcaagtgcaccaaactctttgtggaggccctaggccagatcgggccagcccctcctttgaaggtgcatgttgactgtatgacctcccaaaagcttgtgcgactgccgggattgattgatgtccatgtgcacctgcgggaaccaggtgggacacataaggaggactttgcttcaggcacagccgctgccctggctgggggtatcaccatggtgtgtgccatgcctaatacccggccccccatcattgacgcccctgctctggccctggcccagaagctggcagaggctggcgcccggtgcgactttgcgctattccttggggcctcgtctgaaaatgcaggaaccttgggcaccgtggccgggtctgcagccgggctgaagctttacctcaatgagaccttctctgagctgcggctggacagcgtggtccagtggatggagcatttcgagacatggccctcccacctccccattgtggctcacgcagagcagcaaaccgtggctgctgtcctcatggtggctcagctcactcagcgctcagtgcacatatgtcacgtggcacggaaggaggagatcctgctaattaaagctgcaaaggcacggggcttgccagtgacctgcgaggtggctccccaccacctgttcctaagccatgatgacctggagcgcctggggcctgggaagggggaggtccggcctgagcttggctcccgccaggatgtggaagccctgtgggagaacatggctgtcatcgactgctttgcctcagaccatgctccccataccttggaggagaagtgtgggtccaggcccccacctgggttcccagggttagagaccatgctgccactactcctgacggctgtaagcgagggccggctcagcctggacgacctgctgcagcgattgcaccacaatcctcggcgcatctttcacctgcccccgcaggaggacacctatgtggaggtggatctggagcatgagtggacaattcccagccacatgcccttctccaaggcccactggacaccttttgaagggcagaaagtgaagggcaccgtccgccgtgtggtcctgcgaggggaggttgcctatatcgatgggcaggttctggtacccccgggctatggacaggatgtacggaagtggccacagggggctgttcctcagctcccaccctcagcccctgccactagtgagatgaccacgacacctgaaagaccccgccgtggcatcccagggcttcctgatggccgcttccatctgccgccccgaatccatcgagcctccgacccaggtttgccagctgaggagccaaaggagaagtcctctcggaaggtagccgagccagagctgatgggaacccctgatggcacctgctaccctccaccaccagtaccgagacaggcatctccccagaacctggggacccctggcttgctgcacccccagacctcacccctgctgcactcattagtgggccaacatatcctgtccgtccagcagttcaccaaggatcagatgtctcacctgttcaatgtggcacacacactgcgtatgatggtgcagaaggagcggagcctcgacatcctgaaggggaaggtcatggcctccatgttctatgaagtgagcacacggaccagcagctcctttgcagcagccatggcccggctgggaggtgctgtgctcagcttctcggaagccacatcgtccgtccagaagggcgaatccctggctgactccgtgcagaccatgagctgctatgccgacgtcgtcgtgctccggcacccccagcctggagcagtggagctggccgccaagcactgccggaggccagtgatcaatgctggggatggggtcggagagcaccccacccaggccctgctggacatcttcaccatccgtgaggagctgggaactgtcaatggcatgacgatcacgatggtgggtgacctgaagcacggacgcacagtacattccctggcctgcctgctcacccagtatcgtgtcagcctgcgctacgtggcacctcccagcctgcgcatgccacccactgtgcgggccttcgtggcctcccgcggcaccaagcaggaggaattcgagagcattgaggaggcgctgcctgacactgatgtgctctacatgactcgaatccagaaggaacgatttggctctacccaggagtacgaagcttgctttggtcagttcatcctcactccccacatcatgacccgggccaagaagaagatggtggtgatgcacccgatgccccgtgtcaacgagataagcgtggaagtggactcggatccccgcgcagcctacttccgccaggctgagaacggcatgtacatccgcatggctctgttagccaccgtgctgggccgtttctagggcctggcttcctcagcctcttctctttaggcccagctgctgggcaaggaattccagtgcctcctacgggggcagcacacttagatattcctggacatccagatagctcacatgtgctgaccacacttcaggctctggactggagctctctggcatgggggtggggcctcagatgctggggcccagtctgccccatcttcattcctgcaccttaaacctgtacagtcatttttctactgacttaataaacagccgagctgtcccttgatgctgaaaaaaaaaaaaaaaaaa

SEQ ID NO: 32 - RNA, 7 nt, Artificial sequence
  oligonucleotide sequence that can be used to form the loop in shRNA
caagaga

SEQ ID NO: 33 - RNA, 6 nt, Artificial sequence
  oligonucleotide sequence that can be used to form the loop in shRNA
uuacaa

SEQ ID NO: 34 - RNA, 21 nt, Artificial Sequence
  miR-224-5p- for CAD (MIMAT0000281)
caagucacuagugguuccguu

SEQ ID NO: 35 - RNA, 19 nt, Artificial Sequence
  antisense against citrin
uuaagaaagugcuacgcua

SEQ ID NO: 36 - RNA, 19 nt, Artificial Sequence
  antisense against citrin
caaguuaguuucuccuauu

SEQ ID NO: 37 - RNA, 19 nt, Artificial Sequence
  antisense against citrin
gaucagagccaguuccuaa

SEQ ID NO: 38 - RNA, 19 nt, Artificial Sequence
  antisense against citrin
gaccaaagauggauuaaua

SEQ ID NO: 39 - DNA, 21 bp, Artificial Sequence
  antisense against CAD
ttcaccacacaataatccacg

SEQ ID NO: 40 - DNA, 20 bp, Artificial Sequence
  gRNA  for citrin exon 1
gccatgattcgccccggttg

SEQ ID NO: 41 - DNA, 20 bp, Artificial Sequence
  gRNA for citrin exon 9
tgaccttagcagacattgaa

SEQ ID NO: 42 - protein, 412 aa, Homo sapiens
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 43 - DNA, 1863 bp, Homo sapiens
gccggcgcgcccctgggagggtgagccggcgccgggcccaggcccggacctggtgggaggcggggggaggtggggacgaggcctggggaggcgggccccgcccatctgcaggtggctgtgaacgctgagcggctccaggcgggggccgggcccgggggcggggtctgtggcgcgcgtccccgccacgtgtccccggtcaccggccctgcccccgggccctgtgcttataacctgggatgggcacccctgccagtcctgctctgccgcctgccaccgctgcccgagcccgagtggttcactgcactgtgaaaacagattccagacgccgggaactcacgcctccaatcccagacgctatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatagacccgtgtacaatgaggagctggggcctcctcaatttgcagatcccccaagtacaggcgctaattgttgtgataatttgtaattgtgacttgttctccccggctggcagcgtagtggggctgccaggccccagctttgttccctggtccccctgaagcctgcaaacgttgtcatcgaagggaagggtggggggcagctgcggtggggagctataaaaatgacaattaaaagagacactagtcttttatttctaaaaaaaaaaaaaaaa

SEQ ID NO: 44 - DNA, 1801 bp, Homo sapiens
gccggcgcgcccctgggagggtgagccggcgccgggcccaggcccggacctggtgggaggcggggggaggtggggacgaggcctggggaggcgggccccgcccatctgcaggtggctgtgaacgctgagcggctccaggcgggggccgggcccgggggcggggtctgtggcgcgcgtccccgccacgtgtccccggtcaccggccctgcccccgggccctgtgcttataacctgggatgggcacccctgccagtcctgctctgccgcctgccaccgctgcccgagcccgacgctatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatagacccgtgtacaatgaggagctggggcctcctcaatttgcagatcccccaagtacaggcgctaattgttgtgataatttgtaattgtgacttgttctccccggctggcagcgtagtggggctgccaggccccagctttgttccctggtccccctgaagcctgcaaacgttgtcatcgaagggaagggtggggggcagctgcggtggggagctataaaaatgacaattaaaagagacactagtcttttatttctaaaaaaaaaaaaaaaa

SEQ ID NO: 45 - DNA, 19 bp, Artificial Sequence
  siRNA directed against citrin (sh917)
tgggagaactcatgtataa

(19) United States
(12) Patent Application Publication
(10) Pub. No.: US 2018/0161300 A1
(43) Pub. Date: Jun. 14, 2018

(54) CITRIN INHIBITORS FOR THE TREATMENT OF CANCER

(71) Applicant: Yeda Research And Development Co. Ltd., Rehovot (IL)

(72) Inventors: Ayelet EREZ, Moshav Bnei Zion (IL); Shiran RABINOVICH, Rehovot (IL); Alon SILBERMAN, Rehovot (IL); Shani AGRON, Rehovot (IL)

(73) Assignee: Yeda Research and Development Co., Ltd., Rehovot (IL)

(21) Appl. No.: 15/571,071

(22) PCT Filed: May 10, 2016
(86) PCT No.: PCT/IL2016/050497

§ 371 Date: Nov. 1, 2017

Related U.S. Application Data
(60) Provisional application No. 62/159,489, filed on May 11, 2015.

(51) Int. Cl. A61K 31/216; A61P 35/00; A61K 31/713; A61K 31/513; C12N 15/113; A61K 45/06

(52) U.S. Cl. CPC A61K 31/216; A61P 35/00; A61K 31/713; C12N 2310/531; C12N 15/113; A61K 45/06; C12N 2310/14; A61K 31/513

(57) ABSTRACT

Provided are methods, pharmaceutical compositions and kits for treating cancer in a subject in need thereof, by administering to the subject a therapeutically effective amount of an agent which downregulates a mitochondrial aspartate-dependent pyrimidine synthesis, thereby treating the cancer.

### FIELD AND BACKGROUND OF THE INVENTION

The present invention, in some embodiments thereof, relates to methods, pharmaceutical compositions and kits for treating cancer and, more particularly, but not exclusively, to using an agent which downregulates a mitochondrial aspartate-dependent pyrimidine synthesis for treating the cancer.

Argininosuccinate synthase (ASS1) is a urea cycle cytosolic enzyme that is essential in the conversion of nitrogen from ammonia and aspartate to urea. ASS1 catalyses the condensation of aspartate transported across the mitochondria and citrulline to form argininosuccinate, the immediate precursor of arginine. In the liver, this is a critical step in conversion of nitrogenous waste to urea. Unlike most other urea cycle enzymes that have a limited expression profile, ASS1 is expressed in most tissues where it catalyzes the penultimate step in the de novo synthesis of arginine.

In the cytosol, aspartate serves as a substrate for both ASS1 and the enzymatic complex CAD, i.e., the trifunctional protein carbamoyl-phosphate synthase 2, aspartate transcarbamylase and dihydroorotase. CAD catalyzes the de novo synthesis of pyrimidine nucleotides from glutamine and aspartate, via the formation of dihydroorotate, the substrate for orotic acid.

Citrin [also known as “solute carrier family 25 member 13 (SLC25A13)”, “aralar 2” or “aralar2”] is a mitochondrial carrier protein which catalyzes the exchange of aspartate for glutamate and a proton across the inner mitochondrial membrane, and is stimulated by calcium on the external side of the inner mitochondrial membrane.

Citrullinemia is a urea cycle disorder caused by germline mutations that lead to decreased flux through ASS1. Citrullinemia type I (CLTN I) is caused by ASS1 deficiency and citrullinemia type II (CLTN II) is caused by deficiency in the mitochondrial aspartate transporter Citrin (Dimmock, D. P. et al. 2008; Dimmock, D. et al. 2009).

In contrast to the well-delineated biochemical and clinical consequences of the loss-of-function germline mutations in ASS1, the consequences of somatic downregulation of ASS1 expression in many tumors including melanoma, renal cell carcinoma, hepatocellular carcinoma, prostate carcinoma, mesothelioma, osteosarcoma, and small cell lung cancers, remain obscure (Long, Y. et al. 2013; Delage, B. et al. 2010). Although patients with germline mutations causing ASS1 deficiency have not been reported to have an increased risk for cancer, several studies have shown a correlation between somatic deficiency of ASS1 in cancer and poor survival or increased metastasis, thus supporting a metabolic tumor-suppressive role for ASS1, following the initiation of cancerous proliferation [Huang, H. Y. et al. ASS1 as a novel tumor suppressor gene in myxofibrosarcomas: aberrant loss via epigenetic DNA methylation confers aggressive phenotypes, negative prognostic impact, and therapeutic relevance. Clin Cancer Res 19, 2861-2872, 2013; Kobayashi, E. et al. Reduced Argininosuccinate Synthetase Is a Predictive Biomarker for the Development of Pulmonary Metastasis in Patients with Osteosarcoma. Mol Cancer Ther, 2010; Allen, M. D. et al. Prognostic and therapeutic impact of argininosuccinate synthetase 1 control in bladder cancer as monitored longitudinally by PET imaging. Cancer Res 74, 896-907, 2014].

Argininosuccinate lyase (ASL), the enzyme downstream of ASS1, is directly responsible for arginine synthesis (Morris, S. M., Jr. et al., Br J Pharmacol 157, 922-930, 2009). Recently, it has been shown that ASL has a role in the pathogenesis of renal tumors in fumarate hydratase deficiency, wherein the flux through the enzyme is reversed and arginine is used as a substrate (Zheng, L. et al. 2013; Adam, J. et al. 2013).

A well-established consequence of ASS1 or ASL deficiency is that it leads to arginine auxotrophy, i.e., cells which are dependent on exogenous arginine for growth (Wheatley, D. N 2004) and thus, arginine catabolizing enzymes have been used as therapy in ASS1 depleted tumors. However, such therapy has been of limited benefit, especially in melanoma, wherein the cancer cells develop resistance by re-expressing ASS1 within days of initiation of therapy (Tsai, W. B. et al. 2009).

Several clinical trials in patients with ASS1 deficient hepatocellular carcinoma and mesothelioma combine arginine-depleting agents with thymidylate synthase inhibitors as capecitabine and pemetrexed [clinicaltrials(dot)gov NCT02089633, NCT02029690)].

Delage B., et al., 2010 (Arginine deprivation and argininosuccinate synthetase expression in the treatment of cancer. Int. J. Cancer. 126(12):2762-72) describes approaches in the prevention, diagnosis and treatment of malignant disease based on ASS1 pathophysiology and its rate-limiting product, arginine.

US patent Application No. 20150045248 to Takahashi; Nobuhiro et al. discloses Aralar 2 (SLC25A3; citrin) as a binding protein candidate of TAR DNA-binding protein 43 (TDP-43).

Dong Hui et al., 2011 (“Digital karyotyping reveals probable target genes at 7q21.3 locus in hepatocellular carcinoma”; BMC Medical Genomics 4:60) describe a genomic amplification at 7(421.3 locus which includes the SLC23A13 gene in hepatocellular carcinoma, however, no differences were observed in the expression level of SLC23A13 between tumorous and non tumorous liver tissues.

US Patent Application No. 20140342946 to Kuriakose; Moni Abraham et al. discloses diagnostic tests for predicting prognosis, recurrence, resistance or sensitivity to therapy and metastatic status in cancer.

US patent Application No. 20120192298 A1 to Weinstein; Edward et al. describes methods for genome editing in a non-human subject.

Additional background art include Amoedo ND1, et al. 2016. “AGC1/2, the mitochondrial aspartate-glutamate carriers.” (Biochim Biophys Acta. 2016 Apr. 28. pii:

S0167-4889(16)30092-1 [Epub ahead of print]); US Patent Application No. 20040101874 to Ghosh Soumitra et al.;

### SUMMARY OF THE INVENTION

According to an aspect of some embodiments of the present invention there is provided a method of treating cancer in a subject in need thereof, the method comprising administering to the subject a therapeutically effective amount of an agent which downregulates a mitochondrial aspartate-dependent pyrimidine synthesis, thereby treating the cancer.

According to an aspect of some embodiments of the present invention there is provided a pharmaceutical composition comprising as an active ingredient a therapeutically effective amount of at least two distinct agents and a pharmaceutically acceptable carrier, wherein the at least two distinct agents selected:

down-regulating in tumor cells a mitochondrial aspartate-dependent pyrimidine synthesis; or

at least one of the at least two distinct agents down-regulating in tumor cells a mitochondrial aspartate-dependent pyrimidine synthesis and a second agent of the at least two distinct agents being an agent selected from the group consisting of: an agent for arginine depletion therapy, an agent for glutamine depletion, chemotherapy which inhibits production of nucleotide(s), an agent which inhibits the mammalian target of Rapamycin (mTOR) pathway, a thymidine synthase inhibitor, and an agent which over-expresses the Argininosuccinate synthase polypeptide.

According to an aspect of some embodiments of the present invention there is provided an agent which down-regulates a mitochondrial aspartate-dependent pyrimidine synthesis for treating cancer in a subject in need thereof.

According to an aspect of some embodiments of the present invention there is provided a kit for treating cancer comprising at least two containers, the at least two containers separately packaging at least two distinct agents selected:

down-regulating in tumor cells a mitochondrial aspartate-dependent pyrimidine synthesis; or

at least one of the at least two distinct agents down-regulating in tumor cells a mitochondrial aspartate-dependent pyrimidine synthesis and a second agent of the at least two distinct agents being an agent selected from the group consisting of: an agent for arginine depletion therapy, an agent for glutamine depletion, chemotherapy which inhibits production of nucleotide(s), an agent which inhibits the mammalian target of Rapamycin (mTOR) pathway, a thymidine synthase inhibitor, and an agent which over-expresses the Argininosuccinate synthase polypeptide.

According to some embodiments of the invention, the agent which downregulates a mitochondrial aspartate-dependent pyrimidine synthesis is an agent which downregulates activity or expression of a polypeptide or an enzyme selected from the group consisting of citrin, carbamoyl-phosphate synthase 2, aspartate transcarbamylase and dihydroorotase.

According to some embodiments of the invention, the agent which downregulates a mitochondrial aspartate-dependent pyrimidine synthesis is an agent which downregulates activity or expression of citrin.

According to some embodiments of the invention, the agent which downregulates a mitochondrial aspartate-dependent pyrimidine synthesis is an oligonucleotide.

According to some embodiments of the invention, the agent which downregulates a mitochondrial aspartate-dependent pyrimidine synthesis is a small molecule.

According to some embodiments of the invention, the oligonucleotide is selected from the group consisting of an RNA silencing agent and a genome editing agent.

According to some embodiments of the invention, the agent which downregulates a mitochondrial aspartate-dependent pyrimidine synthesis comprises a plurality of agents for downregulating activity or expression of at least two of the citrin, carbamoyl-phosphate synthase 2, aspartate transcarbamylase and dihydroorotase.

According to some embodiments of the invention, the method further comprising administering to the subject an arginine depletion therapy.

According to some embodiments of the invention, the method further comprising administering to the subject an agent for glutamine depletion.

According to some embodiments of the invention, the method further comprising administering to the subject chemotherapy which inhibits production of nucleotides.

According to some embodiments of the invention, the chemotherapy comprises pyrimidine analog(s), purine analog(s) and/or folate antagonist(s).

According to some embodiments of the invention, the method further comprising administering to the subject an agent which inhibits the mammalian target of Rapamycin (mTOR) pathway.

According to some embodiments of the invention, the agent is Rapamycin, rapalogs (rapamycin derivatives) and/or mTORC1/mTORC2

According to some embodiments of the invention, the method further comprising administering to the subject a thymidine synthase inhibitor.

According to some embodiments of the invention, the thymidine synthase inhibitor is Fluorouracil (5-FU).

According to some embodiments of the invention, the method further comprising over-expressing within tumor cells of the subject the Argininosuccinate synthase polypeptide.

According to some embodiments of the invention, the cancer is characterized by an increased level of citrin as compared to the level of the citrin in a non-malignant tissue of the same origin as the cancer.

According to some embodiments of the invention, the cancer is characterized by a decreased level of Argininosuccinate synthase as compared to the level of the Argininosuccinate synthase in a non-malignant tissue of the same origin as the cancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-K demonstrate that ASS1 inactivation correlates with noncancerous proliferation. FIG. 1A—A schematic illustration of the metabolic flux involved in nitrogen contributions to nucleic acid synthesis either directly from glutamine or through aspartate. The aspartate nitrogen can be used for synthesis of pyrimidines (green path) or urea (blue path). CTLN I is caused by ASS1 deficiency whereas CTLN II is caused due to deficiency of citrin. While both conditions are characterized by elevations of citrulline and ammonia in the plasma, in ASS1 deficiency there is a potential diversion of the aspartate towards pyrimidine whereas this is not the case with citrin deficiency wherein the aspartate is not transported across the mitochondria. ASS1—argininosuccinate synthase; ASL—argininosuccinate lyase; ASA-argininosuccinate; ARG1—arginase; CAD-carbamoyl-phosphate synthase 2, aspartate transcarbamylase and dihydroorotase. FIG. 1B—Effect of ASS1 activation on the cell growth rate and on the flux through the reaction catalyzed by CAD, as predicted by the generic human model; decreasing ASS1 activation (green line) results in an increase in both the cellular growth rate and in the flux through the CAD reaction (blue line), A.U.—arbitrary units. FIG. 1C—Urinary orotic acid levels are elevated significantly in patients with CTLN I as compared to normative values in control subjects (0.3 up to 2.8 mmol/mol creatinine, depicted by the red dashed line), and to those with CTLN II, *p-Value<0.01 using Student T-Test analysis. (n=5 with CTLN I and 4 with CTLN II). FIG. 1D—Pyrimidine measurement using LC/MS shows significantly lower levels in CTLN II fibroblasts as compared to CTLN I and to normal fibroblasts. TMP was undetectable (i.e., below detection levels). The experiment was repeated three times with normal fibroblasts from three different control subjects, pooled cells from two patients with CTLN II and from one subject with CTLN I. FIG. 1E (Upper panel)—Crystal violet staining shows increased proliferation in CTLN I (two right columns of wells) as compared to CTLN II (middle two columns of wells) and to normal control (two left columns of wells). Cells were plated at 150,000/well confluence and stained for crystal violet on day 5. FIG. 1E (Lower panel)—A quantification graph for the staining shown in the upper panel. The experiment was repeated twice with pooled cells from two patients with CTLN II, from one subject with CTLN I and from three control subjects, **p-Value<0.0005; ***p-Value<0.00005. FIGS. 1F-G—Primary fibroblasts from CTLN I subjects have an increased total aspartate (FIG. 1F) and total uracil (FIG. 1G) synthesized from ¹⁵N₅-alpha-glutamine as compared to fibroblasts from patients with CTLN II and to normal controls (*p<0.05). This is a representative graph of three independent experiments using pooled cells from two subjects with CTLN II, from one subject with CTLN I and from three control subjects. FIGS. 1H-K—Mouse intestinal studies show increased Ass1 expression in differentiating cells and decreased expression in proliferating cells. FIGS. 1H-I—A fixed frozen small-intestine tissue section of a 6-day old C57Bl6 mouse was hybridized with single molecule FISH probes libraries for single Ass1 mRNA molecules (green dots) and for Ki67 mRNA molecules (red dots). Nuclei were stained with DAPI (blue). Dashed line outlines the crypt bottom. White lines mark cell borders according to co-immunofluorescence staining with FITC-E-cadherin. A magnifying window (FIG. 1I) highlights a clear demarcation of Ass1 mRNA localizing to the differentiated cells of the crypt and its absence in the proliferating cells which are distinguished by Ki67 mRNA expression (Magnification 100×). Goblet cells exhibit some non-specific fluorescence appearing in multiple channels. FIGS. 1J-K—Quantified correlations between Ass1 and Glut2, an enterocyte marker (FIG. 1J), and between Ass1 and Ki67 (FIG. 1K). The negative correlation between Ki67 and Ass1 (R=−0.61) and the positive correlation between Glut2 and Ass1 (R=0.91) are significant (p-value<10⁻³⁶ for Glut2 and p-value<10⁻⁸ for Ki67). Each dot in these figures represents a cell and the quantification dots were counted on 8 Z-stacks spaced 0.3 μm apart (total tissue volume of 2.4 μm).

FIGS. 2A-H demonstrate that ASS1-deficient tumors have increased proliferation rate and an increase in pyrimidine levels. FIG. 2A—ASS1 is ranked within the top 24 genes out of 14,000, that show a significant inverse correlation with proliferation rate among the 16 NCI-60 cancer cell lines in which ASS1 is downregulated; Shown is a Spearman rank correlation between the expression of each gene and its associated proliferation rates. FIG. 2B—A magnifying view of the correlation between proliferation rate and ASS1 expression levels. FIG. 2C—Predicted differences in the production rate of biomass components following the inactivation of ASS1. The production of nucleic acids marked in arrows, is predicted to have a large increase following ASS1 inactivation in the majority of the NCI-60 cell-lines. The Figure represents the results obtained using the LOX IMVI cell-line model, (*p-value<0.05). FIG. 2D—Analysis of the TCGA database of matched tumor-normal tissue pairs showing that CAD expression is elevated significantly in tumors with ASS1 downregulation as compared to normal tissue, (p-Value=0.0005). FIG. 2E—An immunoblot of osteosarcoma cell lines showing decreased expression of ASS1 compared to the loading control GAPDH in MNNG/HOS as compared to U2OS. FIG. 2F—Osteosarcoma cells with ASS1 downregulation have a significant increase in pyrimidine levels as measured by LC/MS, (*p-Value<0.05; ***p-Value<0.0005). FIG. 2G—Osteosarcoma cells with ASS1 down regulation have a significant increase in total uracil from ¹⁵N₅ alpha glutamine, (*p-Value<0.05). FIG. 2H—Osteosarcoma cells with ASS1 downregulation have a significant increase in proliferation as measured by MTT assay (**p-Value<0.005; ***p-Value<0.0005).

FIGS. 3A-M demonstrate that ASS1 expression levels in cancer determine aspartate availability for pyrimidine synthesis. FIGS. 3A-B—Immunoblots of osteosarcoma cells following transduction with either ASS1 overexpression construct (FIG. 3A) or with ASS1-shRNA (FIG. 3B). FIGS. 3C-D—MTT proliferation assay showing a significant decrease following ASS1 overexpression (OE) (FIG. 3C) and a significant increase in proliferation following transduction with shASS1 (FIG. 3D) in osteosarcoma cells. The proliferation values are shown for day 3, after normalizing the data for the reading on day 1 (*p-Value<0.05; ***p-Value<0.0005). FIG. 3E—LC/MS measurements of pyrimidine levels showing a significant increase following the use of ASS1-shRNA in osteosarcoma cells. FIG. 3F—Total uracil is decreased significantly in osteosarcoma cells with ASS1 overexpression (OE) (*p-Value=0.05). FIG. 3G—dNTP supplementation rescues proliferation after ASS1 overexpression in osteosarcoma cells, (*p-Value=0.01; **p-Value=0.002). FIG. 3H—Pyrimidines nucleotides significantly rescue proliferation in ASS1 overexpressing MNNG cells. FIG. 3I—Pyrimidines significantly rescue proliferation in ASS1 overexpressing MNNG/HOS cells, n≥3, (*p-Value<0.05, **p-Value***, 0.005>p-Value<0.0005) FIGS. 3J-M—10⁷ MALME-3m melanoma cells transduced with either pLKO empty vector or with shASS1, were injected subcutaneously to immune deficient mice. The experiment was repeated twice. Two weeks after injection, the group injected with melanoma cells expressing shASS1 developed tumors that grew more rapidly in size (FIG. 3J) and volume (FIG. 3K) and had higher levels of total aspartate (FIG. 3L) and total uracil (FIG. 3M) as compared to tumors expressing the empty vector (*p-Value≤0.05; **p-Value=0.007, ***p-Value=0.0009).

FIGS. 4A-J demonstrate that decreasing CAD activation decreases proliferation in ASS1-deficient cancers. FIG. 4A—MTT assay showing that decreasing citrin levels significantly decreases proliferation in U2OS osteosarcoma even after a significant proliferation increase is accomplished by ASS1 downregulation (*p-Value=0.05; **p-Value=0.005; ***p-Value<0.0005; ****p-Value<0.00005). FIG. 4B—Decreasing citrin levels decreases pyrimidine levels in U2OS cells with ASS1 downregulation, (*p-Value<0.05; **p-Value<0.009; ***p-Value=0.0008). FIGS. 4C-D—GCMS measurements showing that U2OS with shASS1 has a significant increase in total aspartate levels (FIG. 4C), as well as in total orotic acid (FIG. 4D), that are reversed when transfected with si-citrin (*p-Value<0.05; **p-Value<0.005). FIGS. 4E-F—Kaplan-Meier (KM) survival analysis for 2 different cancer types: BLCA—bladder cancer (FIG. 4E), and LUAD—lung adenocarcinoma (FIG. 4F), showing significant poor survival for cancers with low ASS1 and high citrin expression levels. For each cancer type, the KM-plot indicates the survival rates of 4 patients group: ASS1 low expression—citrin high expression; ASS1 low expression alone; citrin high expression alone; none of the above groups. Analysis was performed for the cancer types for which there was sufficient survival data; **Log rank p-value<0.006 is shown for both cancers between the group of low ASS1 and high citrin to the group with normal expression levels of these genes as well as for the group of low ASS1 and high citrin as compared to only high expression level of citrin in LUAC. FIG. 4G—Immunoblot of osteosarcoma cells for the mTOR pathway downstream effectors—S6K1 and CAD showing increase phosphorylation following shASS1 that is reversed when cells are transfected with si-citrin. FIG. 4H—A quantification graph of crystal violet staining of osteosarcoma cells transduced with shASS1 following drug treatments. All treatments were significant as compared to no treatment. In addition, the results show a significant additive beneficial effect of decreased proliferation in response to treatment with medium depleted of arginine together with either mTOR or pyrimidine synthesis inhibitors (Rapamycin or 5FU respectively). Of note, as isolated treatment, 5FU was more beneficial than Rapamycin. Cells were grown in medium depleted of arginine, in complete medium with either Rapamycin or 5FU, and in arginine depleted medium together with either Rapamycin or 5FU, n=9***p-value<0.0005). FIG. 4I—A western blot showing decreased activation of the mTOR proteins following Rapamycin treatment. FIG. 4J-A Schematic presentation for potential interventions in pyrimidine synthesis in ASS1 deficient tumors; inhibiting citrin-derived aspartate, blocking the mTOR pathway and inhibiting thymidylate synthase, all result in decreased pyrimidine synthesis.

FIGS. 5A-J demonstrate that ASS1 deficiency in cancer correlates with aspartate utilization by CAD. FIG. 5A—Schematic flux tracing of the Alpha labeled nitrogen of glutamine to nucleic acid synthesis via aspartate. FIGS. 5B-C—Labeled levels of M+1 aspartate (FIG. 5B) and M+1 uracil (FIG. 5C) synthesized from 15N-α-labeled glutamine, are higher in fibroblasts from CTLN I as compared to fibroblasts from controls and CTLNII patients, n≥3. Filled bars (grey)=CTLNI; Empty bars (white)—control (normal fibroblasts); Dashed bars=CTLNII; FIG. 5D—The ratio between M+1 labeled/total level of uracil in fibroblasts is similar between citrullinemia patients and control, n□3. Error bars represent SER. FIG. 5E—TCGA analysis of tumor-normal paired tissues for gene expression comparison shows the expression levels of ASL and ASS1 in different cancers. FIG. 5F—A graph plot generated from the modeling data for the production capacity of metabolites following ASS1 inactivation in each of the NCI-60 cell lines as well as in the generic model. The reddish bars represent the ranking of nucleic acids while the blueish bars represent the ranking of all other metabolites. FIG. 5G—Correlation analysis of NCI-60 cell lines shows a significant inverse correlation between ASS1 and CAD expression levels. FIGS. 5H-I—Osteosarcoma (FIG. 5H) and melanoma (FIG. 5I) microarray data was obtained from the NCBI GEO database (accessions GSE33383 and GSE46517, respectively). Raw expression levels were plotted and significance was computed using t-test on log 2-transformed expression levels. The number of patients for each subtype is shown in parenthesis on the left. FIG. 5J—A western blot for CAD and ASS1 shows higher expression level of CAD in MNNG/HOS human osteosarcoma cell line which has low expression level of ASS1 in comparison to U2OS which has higher expression levels of ASS1. p97 is shown as loading control.

FIGS. 6A-L demonstrate that ASS1 inactivation in melanoma correlates with increased proliferation. FIG. 6A—An immunoblot showing different expression levels of ASS1 and CAD in two different cancer cell lines of melanoma. FIG. 6B—Melanoma cells with ASS1 downregulation (LOX-IMVI) have a significant increase in pyrimidine levels as measured by LC/MS. FIG. 6C—Melanoma cells with ASS1 downregulation have a significant increase in total Uracil, n=4. FIG. 6D—Melanoma cells with ASS1 downregulation (LOX-IMVI) have a significant increase in proliferation as measured by MTT assay. FIG. 6E-F—Immunoblots of melanoma cells for ASS1 levels following transduction with either ASS1 over expression (OE) construct (FIG. 6E) or with shASS1 (FIG. 6F). FIG. 6G—Proliferation assays showing a significant decrease in proliferation following ASS1 overexpression (OE) in melanoma using MTT, n=3; FIG. 6H—Crystal violet quantification for melanoma cells following transduction with shASS1 demonstrating increase in proliferation, n=3. FIG. 6I—LC/MS measurements of pyrimidine levels showing a significant increase following the use of shASS1 in melanoma cells, n≥3. FIGS. 6J-K—Uracil M+1 levels are decreased significantly in melanoma cells with ASS1 over expression (OE; FIG. 6J, n≥2) and increased in melanoma cells with shASS1 (FIG. 6K). FIG. 6L—Significant increase in proliferation of melanoma cells by dNTP's after ASS1 overexpression, n=3. In all panels, error bars represent SER.

FIGS. 7A-H demonstrate that downregulation of ASS1 levels increases pyrimidine synthesis. FIGS. 7A-C—Osteosarcoma cells were infected with two different shASS1 vectors: shASS742 and shASS745. Both clones decreased ASS1 levels efficiently to approximately 20% expression (FIG. 7A) resulting in a significant increase in uracil M+1 levels (FIG. 7B) and in proliferation, n≥3 (FIG. 7C). FIG. 7D—RNA levels measured in U2OS at 24 hour intervals show increased levels of RNA in U2OS infected with shASS1 as compared to the empty vector. FIG. 7E—Uracil M+1 levels increase more in U2OS infected with shASS1 as compared to the empty vector during 38 hours of measurements. FIG. 7F The levels of total and labeled M+1 alanine synthesis from ¹⁵N-α-glutamine does not change significantly following ASS1 downregulation, n=3. FIGS. 7G-H—Tumors with shASS1 had higher levels of M+1 aspartate (FIG. 7G) and M+1 uracil (FIG. 7H) synthesized from ¹⁵Nα-glutamine, as compared to tumors expressing the empty vector, n=15. In all panels, error bars represent SER.

FIGS. 8A-N demonstrate that cancers with ASS1 downregulation are addicted to aspartate. FIG. 8A-B—Analysis of the TCGA data base of matched tumor-normal pairs showing no significant difference in the expression level of citrin in tissues with a high base line expression of citrin (FIG. 8A) and significant elevation in tumors in which the normal tissue has low basal expression of citrin (FIG. 8B) (*p<0.001).

FIG. 8C—Immunoblot showing the expression level of citrin in osteosarcoma cells following si-citrin. FIGS. 8D-E—Labeled and unlabeled aspartate (FIG. 8D) and uracil (FIG. 8E) are elevated significantly in cancers with ASS1 downregulation and are comparable to control in cells with both ASS1 and Citrin downregulation; n≥3. Error bars represent SER. FIGS. 8F-G—Kaplan-Meier (KM) survival analysis for 2 different cancer types; BRCA—breast cancer (FIG. 8D) and LUSC—lung squamous cell carcinoma (FIG. 8E), showing poor survival trend for cancers with low ASS1 and high citrin. For each cancer type the KM-plot indicates the survival rates of 4 patients group: ASS1 low expression—citrin high expression; ASS1 low expression; citrin high expression; none of the above groups. Analysis was performed for the cancer types for which there was sufficient survival data. FIGS. 8H-I—Quantification graphs of a Western blot showing decreased CAD (FIG. 8I) and S6K phosphorylation (FIG. 8H) following treatment of U2OS with si-citrine. Error bars represent SER. FIGS. 8J-N—Proximity ligation assay showing increased proximity between CAD and citrin following ASS1 knockdown in U2OS cells (FIG. 8L). FIGS. 8J-K—show the proximity between ASS1 and CAD to citrin in U2OS infected with empty vector while FIG. 8L shows the proximity between CAD and citrin following infection of U2OS with shASS1. Blue nuclei=DAPI staining; Red dots=indication for proximity; FIGS. 8M-N—Quantification of proximity ligation assays performed on U2OS infected with either empty vector (EV) or with shASS1 using antibodies for citrin, ASS1 and CAD. The pictures were quantified using ImageJ.

FIGS. 9A-C demonstrate that citrin deficient patients have decreased glycolysis, NADH and ATP production. FIG. 9A—GC/MS results showing that Citrin deficient cells produce significantly less lactate reflecting decreased glycolysis as compared to control fibroblasts. FIG. 9B—Image stream results showing decreased mitochondrial NADH production by citrin deficient fibroblasts as compared to control fibroblasts. FIG. 9C—A quantitative ATP assay showing that Citrin deficient cells produce less ATP reflecting less oxydative phosphorylation compared to control fibroblasts.

FIGS. 10A-B demonstrate that citrin deficient cells might have increased autophagy. Electron microscope (EM) imaging showing increased autophagosomes in Citrin deficient patients' fibroblasts (CTLN II fibroblasts, FIG. 10B) as compared to control fibroblasts (FIG. 10A).

FIGS. 11A-C demonstrate that citrin silencing decreases cancer cells' proliferation (using MDA-MB-435 melanoma cancer cell-line). FIG. 11A—A western blot showing decreased Citrin protein expression following infection with shCitrin. FIG. 11B—A quantitative RT showing decreased Citrin RNA expression following transfection with shCitrin (compare the columns with Dox as compared to without Dox). FIG. 11C—Crystal violet staining showing decreased proliferation of cells infected with shCitrin as compared to control cells infected with an empty vector (EV). In all panels, induction of shCitrin expression was achieved using Doxycycline (DOX).

FIG. 12 shows that citrin is over expressed in various types of cancer. Computational analysis of Citrin expression in normal and cancerous cells and tissues.

FIGS. 13A-B demonstrate that citrin overexpression increases glycolysis. FIG. 13A—A western blot showing increased Citrin expression following transfection of cancer cells with Citrin plasmid as compared to transfection with empty vector (EV). FIG. 13B—GC/MS results showing increased lactate production reflecting increased glycolysis in cells over expressing Citrin as compared to controls.

FIGS. 14A-D demonstrate that citrin overexpressing cancer cells have enlarged mitochondrias. Electron microscope (EM) imaging showing enlarged mitochondrias in Citrin over expressing cells (FIGS. 14C and 14D) as compared to control cells transfected with empty vector (EV) (FIGS. 14A and 14B).

FIGS. 15A-B depict levels of aspartate M+1 (FIG. 15A) or Uracil M+1 (FIG. 15B) in osteosarcoma cell line (U2OS) incubated with 4 mM of L-Glutamine (ALPHA-15N, 98%) in the presence of either control (medium only) or one of compounds “1”, “2”, or “3”, for 24 hours. Compound “1”=D-Glutamic acid; compound “2”=L-glutamic acid-5-methyl ester; “compound “3”=L-glutamic acid-gamma-benzyl ester. Note the decrease in aspartate and uracil production by the cancerous cells treated with compound “3” (L-glutamic acid-gamma-benzyl ester).

### DESCRIPTION OF SPECIFIC EMBODIMENTS OF THE INVENTION

The present invention, in some embodiments thereof, relates to methods, pharmaceutical compositions and kits for treating cancer and, more particularly, but not exclusively, to the use of an agent which downregulates a mitochondrial aspartate-dependent pyrimidine synthesis for treating the cancer.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

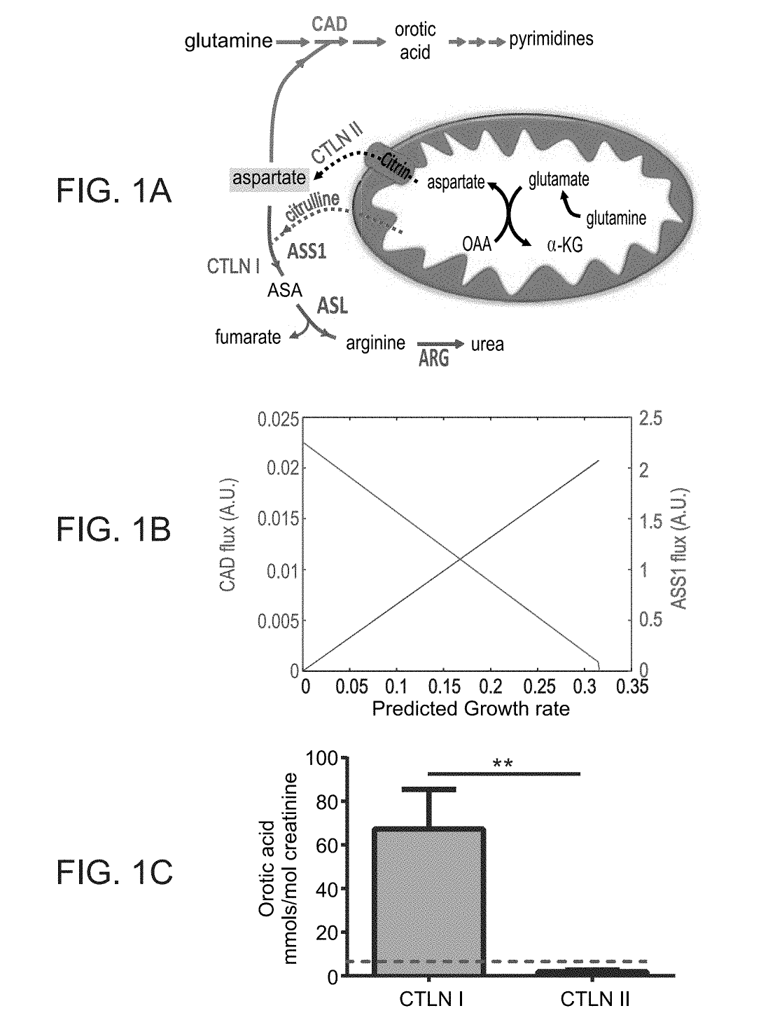

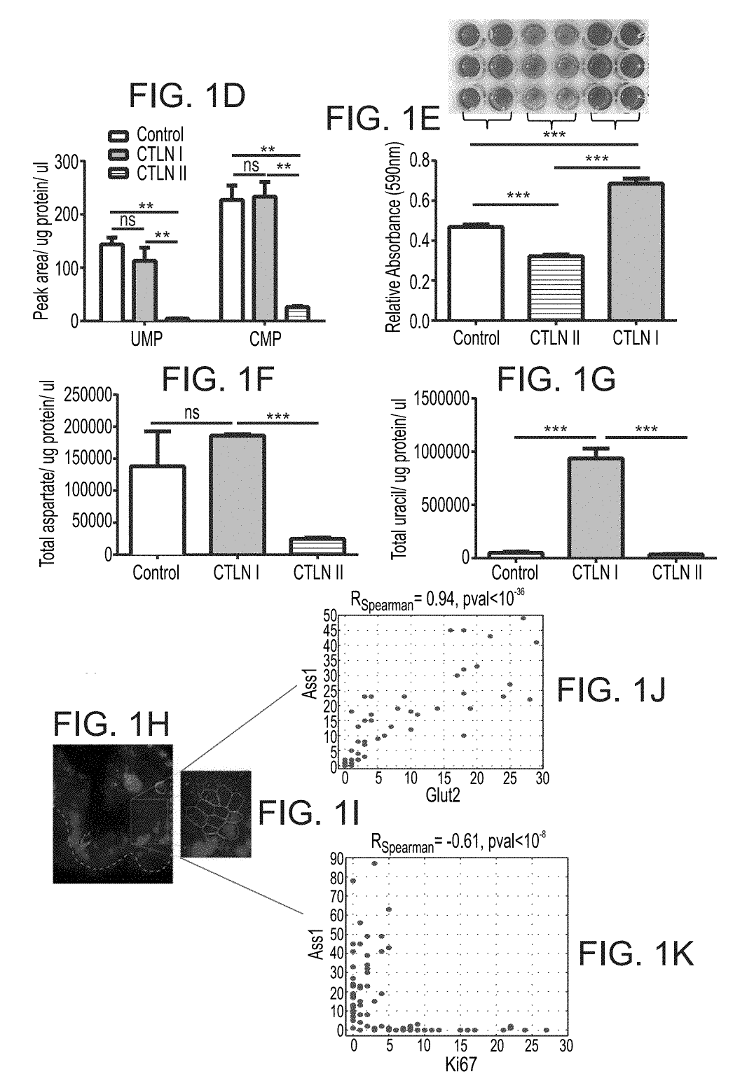

Although ASS1 and ASL participate in the same pathway aimed at arginine synthesis, there are cancers in which both genes are epigenetically silenced (Syed, N. et al. 2013). Whereas ASS1 silencing renders the tumors auxotrophic for arginine, without being bound by any theory, the present inventors have hypothesized that down-regulation of ASS1 has an arginine-independent survival effect by redirecting of aspartate towards pyrimidine synthesis (FIG. 1A).

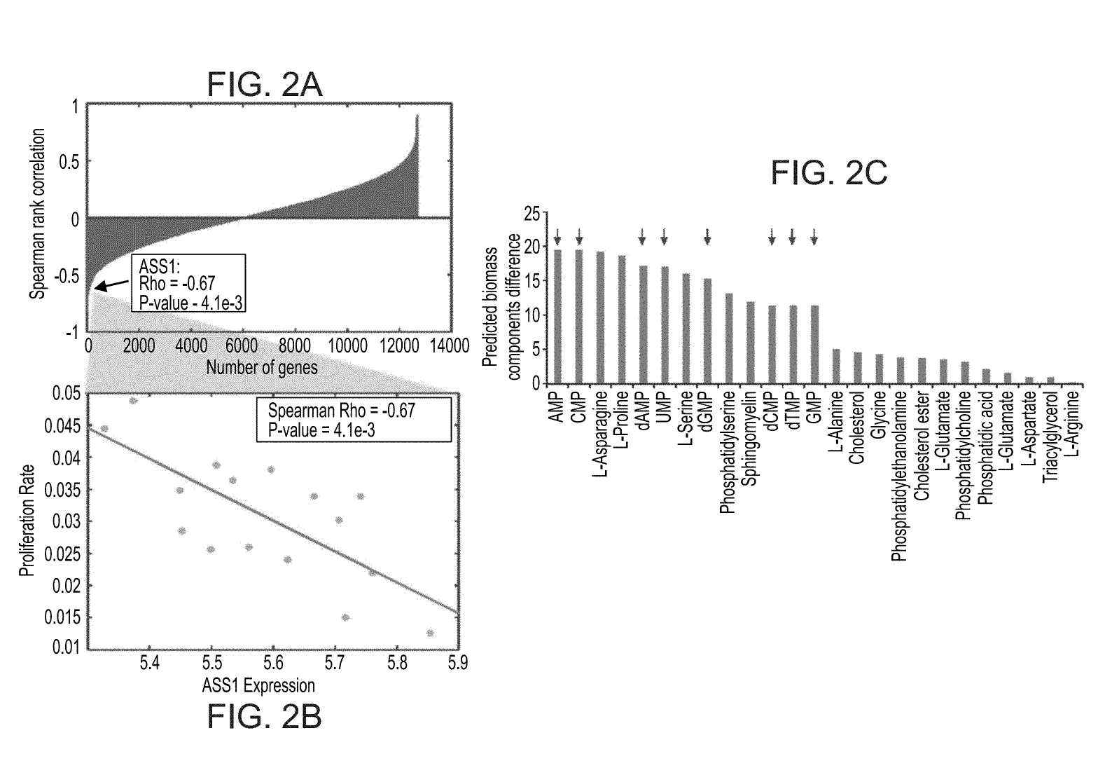

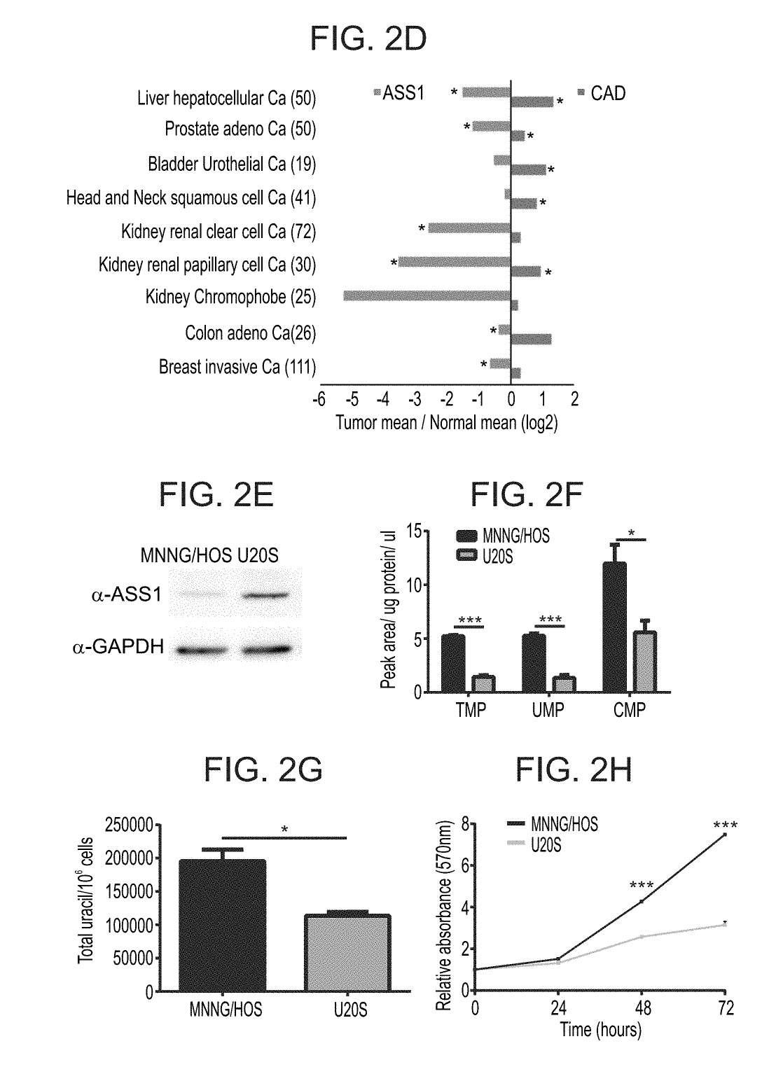

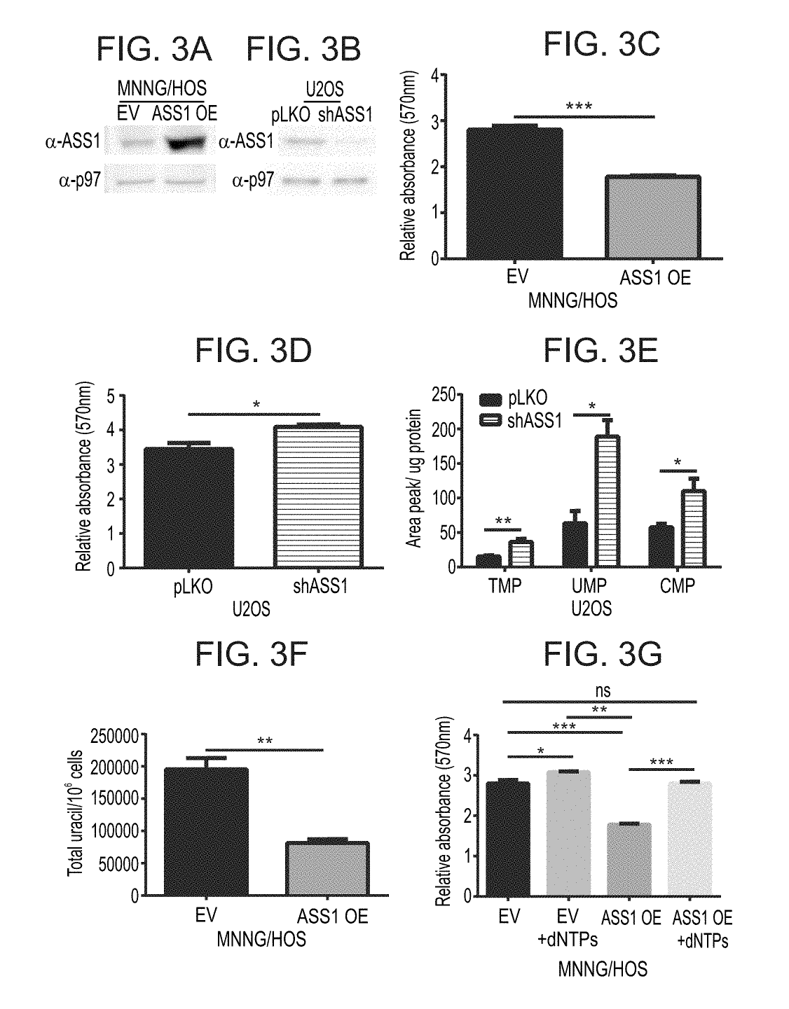

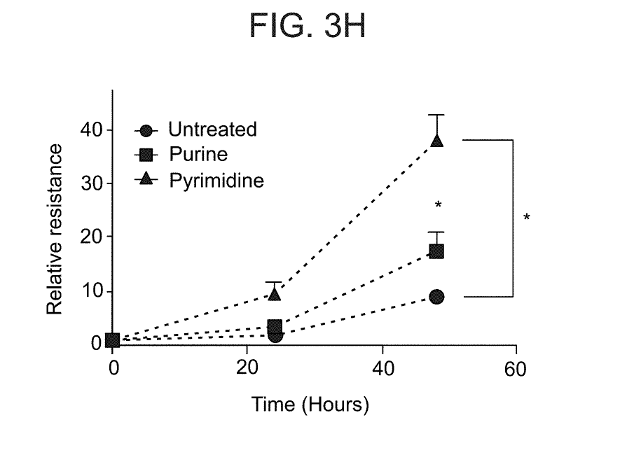

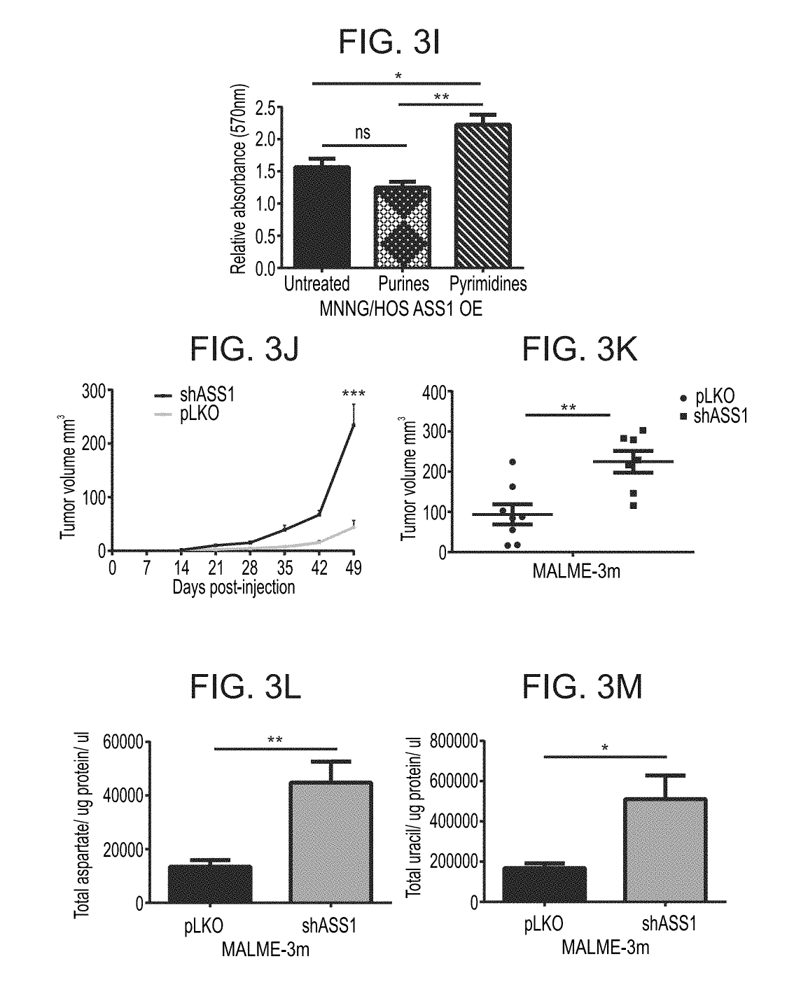

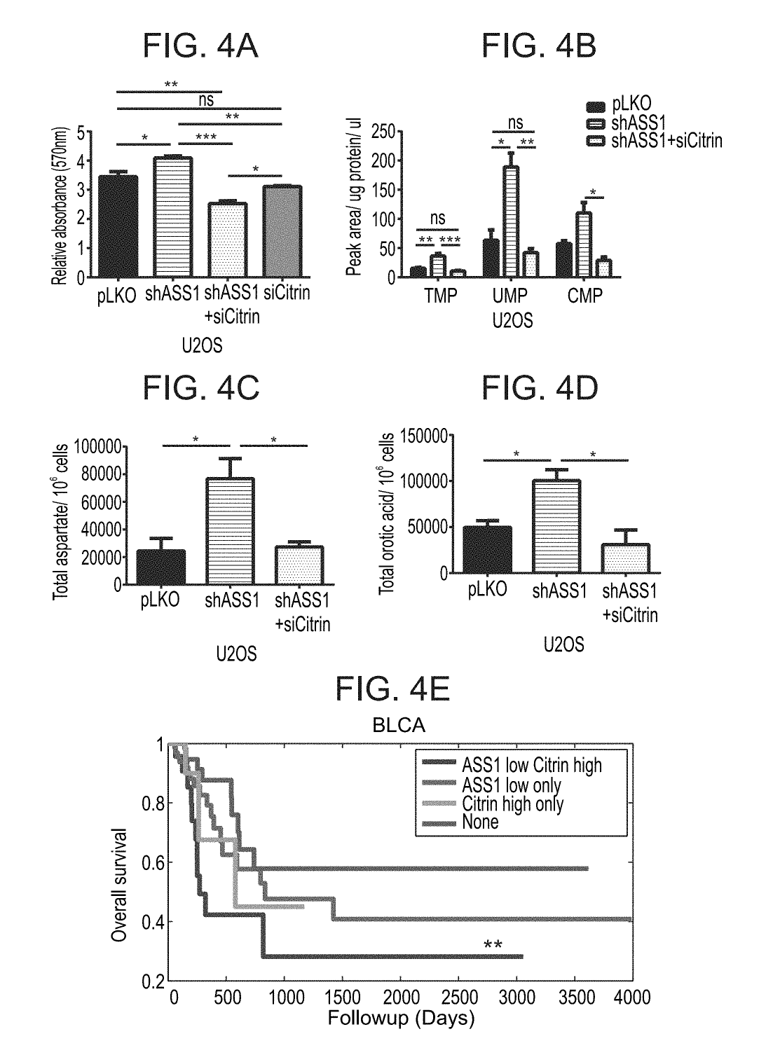

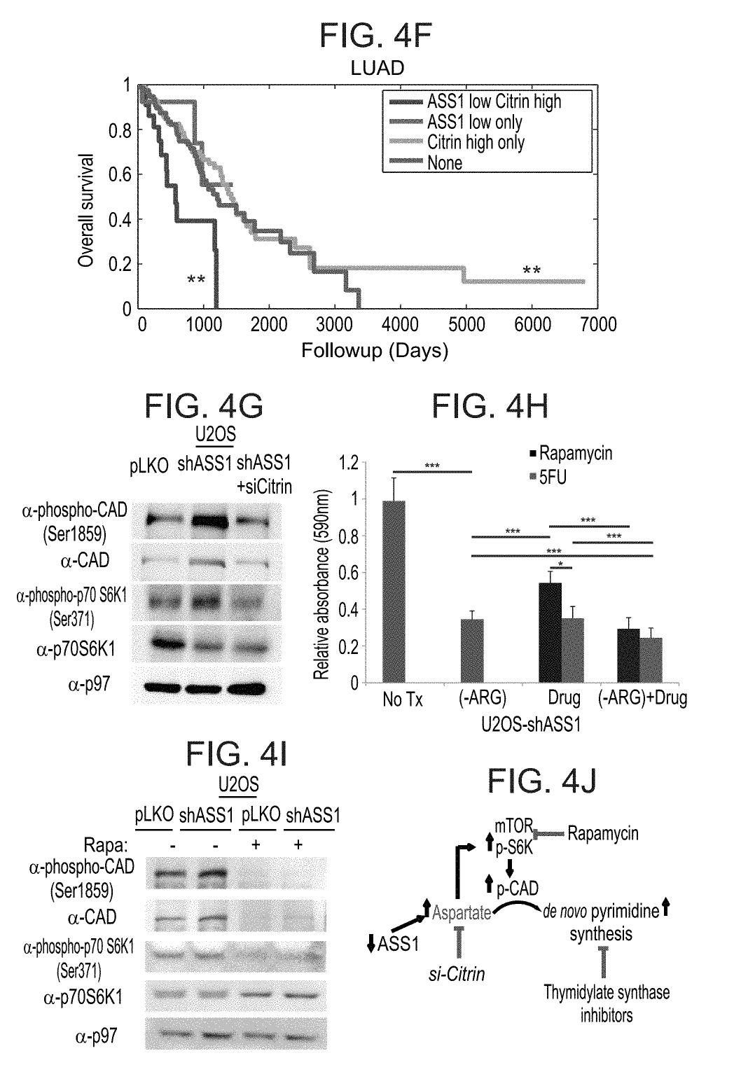

Thus, the present inventors studied the consequences of loss of ASS1 activation in human, in the two types of citrullinemia and found that in citrullinemia type I (CTLN I), which is caused by enzymatic deficiency of ASS1, there is an increased pyrimidine synthesis and proliferation as compared to citrullinemia type II (CTLN II), in which there is a decreased substrate availability for ASS1 due to deficiency of the aspartate transporter, citrin (SLC25A13). The present inventors further demonstrate that ASS1 deficiency in cancer increases cytosolic aspartate levels (FIGS. 2A-H and FIGS. 3A-M) which increases CAD activation by upregulating its substrate availability and also by increasing its phosphorylation by S6K1 through the mammalian target of Rapamycin (mTOR) pathway (FIGS. 4A-J). Decreasing CAD activity by blocking citrin, the mTOR signaling of pyrimidine synthesis, decreases proliferation and thus may serve as a therapeutic strategy in multiple cancers where ASS1 is downregulated.

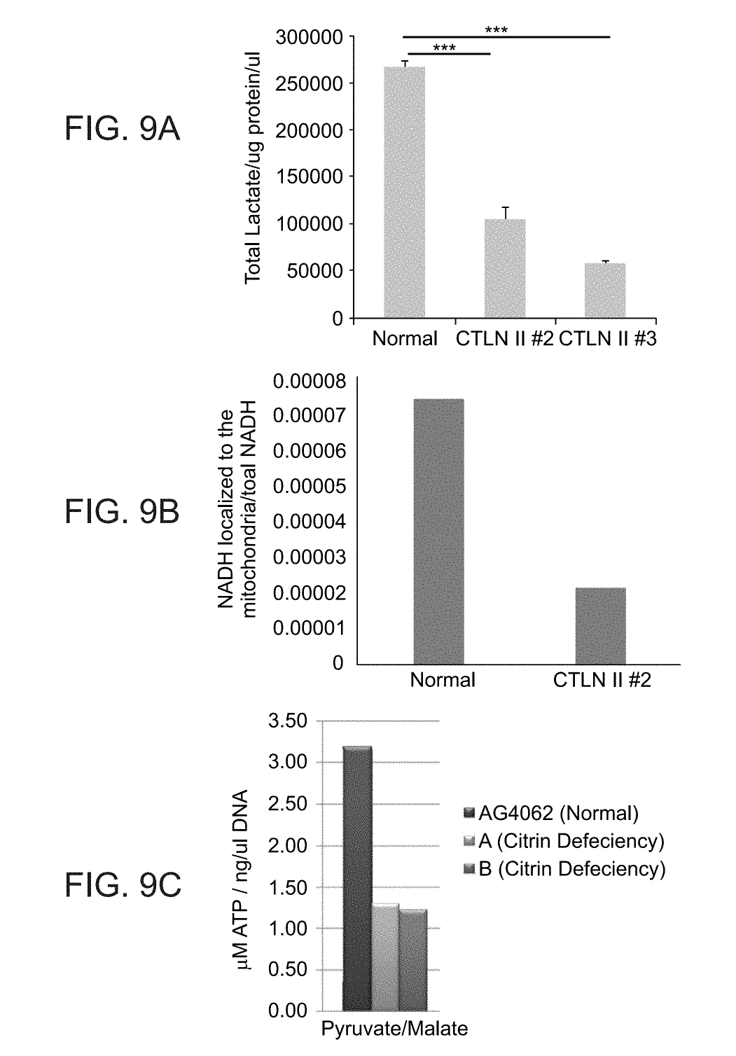

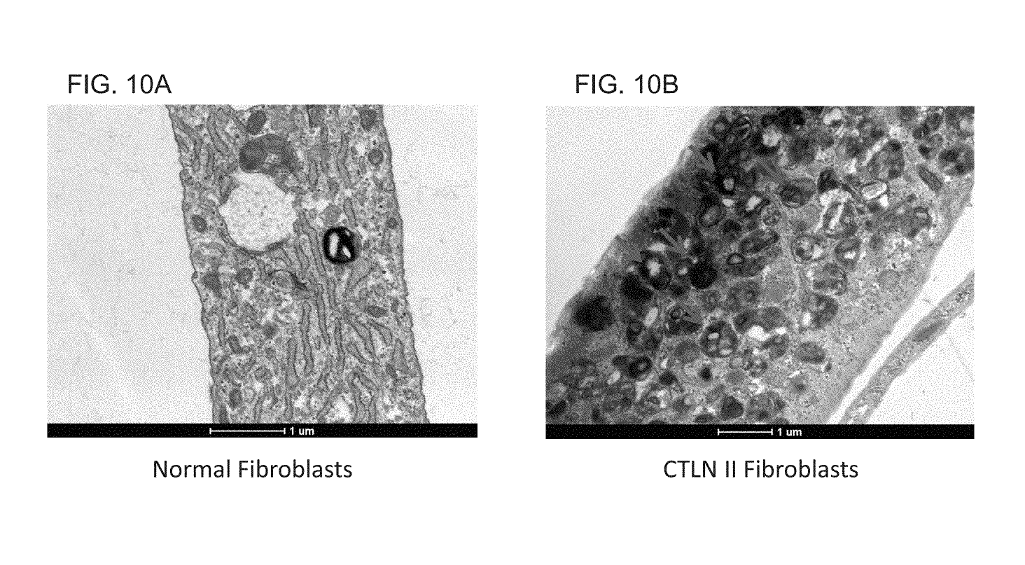

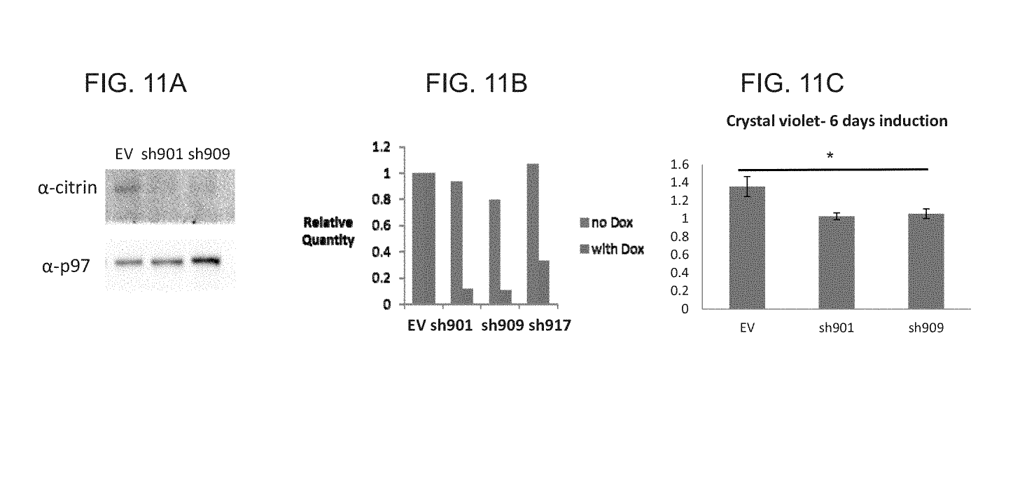

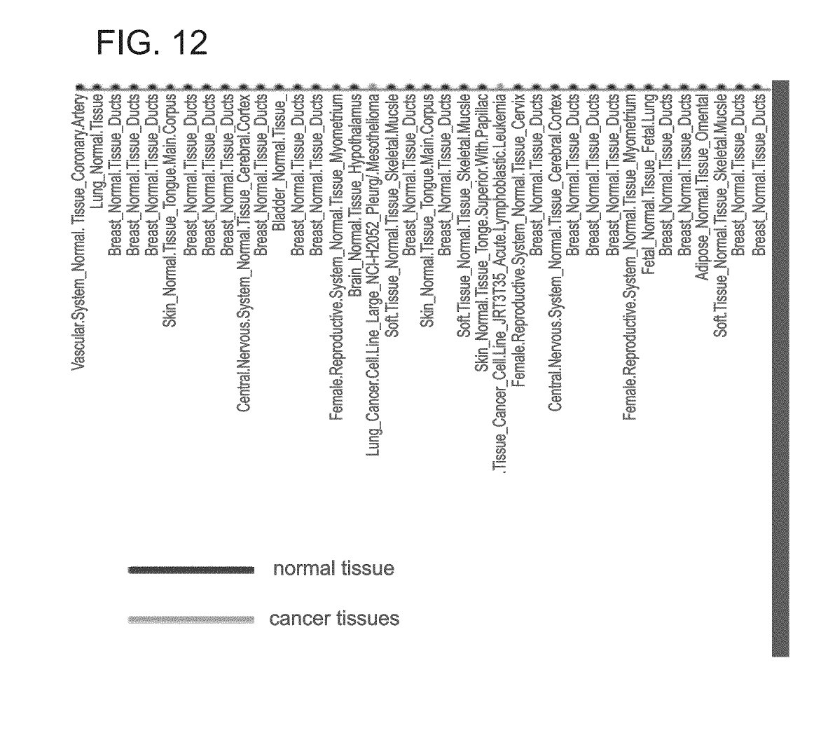

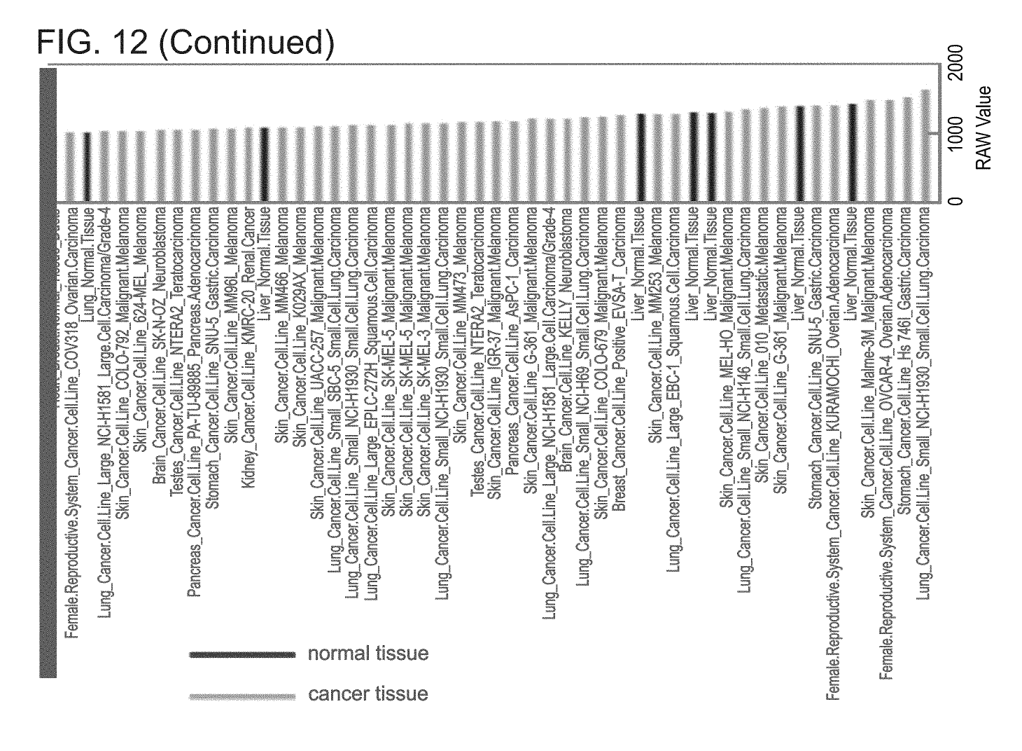

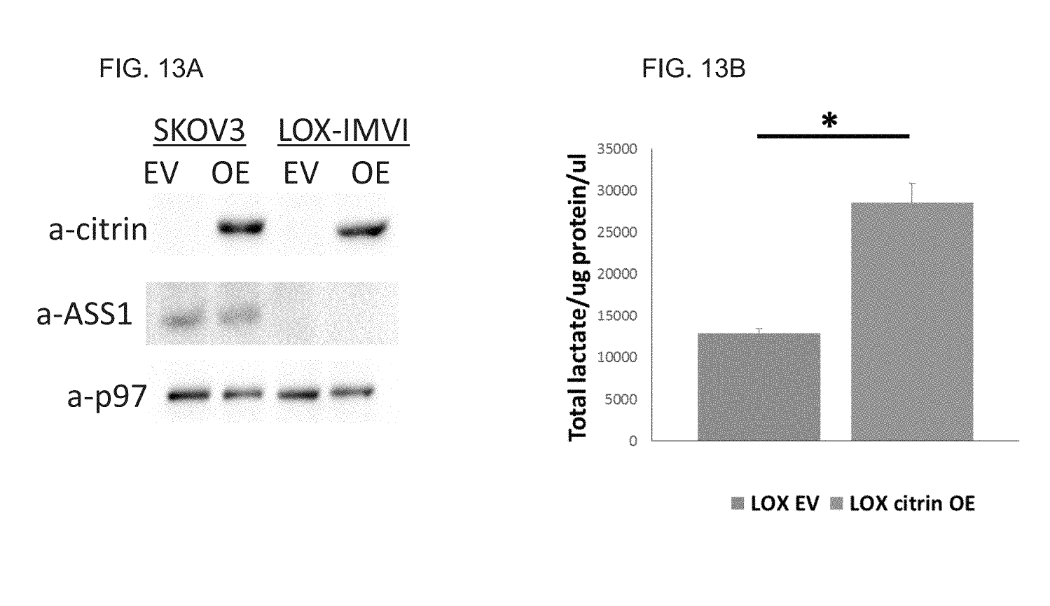

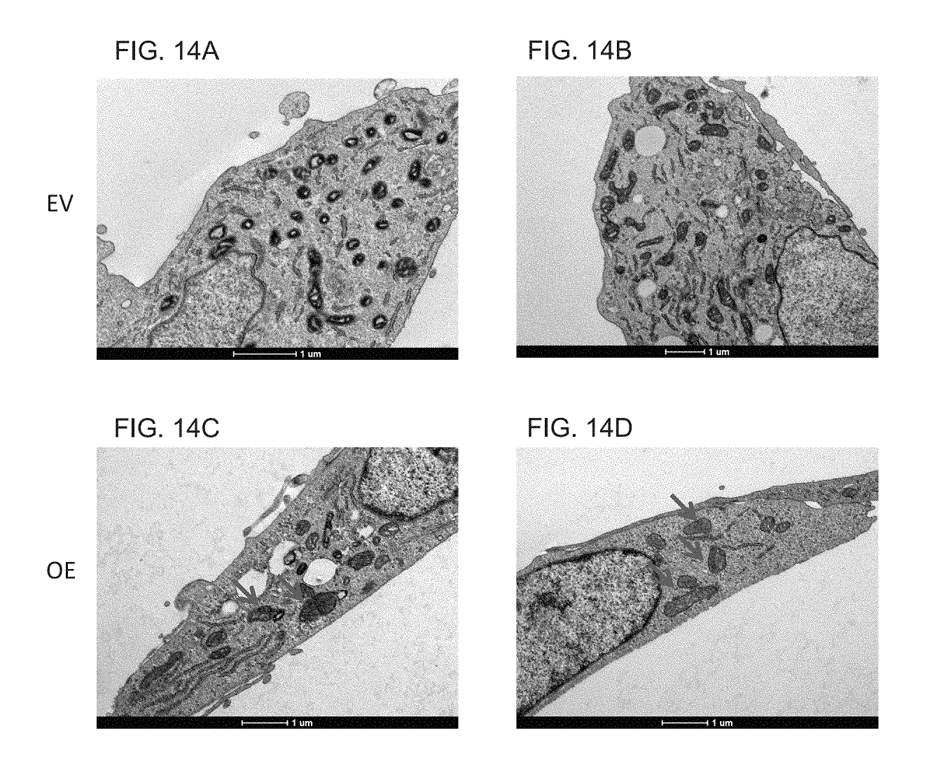

Thus, supported by computational modeling and using multiple methodologies including studies of fibroblasts from patients with CLTN I and CLTN II, cancer cells, clinical data, robust informatics analysis of multiple tumors, the present inventors show that ASS1 is a key regulator of the mitochondria-derived aspartate flux. Silencing of ASS1, leads to preferential diversion of aspartate away from the synthesis of arginine and urea to pyrimidine synthesis. Decreasing aspartate flux to pyrimidine synthesis by either expressing ASS1 in cancer cells that have endogenous silencing, or by blocking the transport of aspartate through the mitochondrial membrane by inhibiting Citrin, decreases cell proliferation due to decreased nucleic acid synthesis. The results demonstrate that ASS1 silencing is a novel mechanism to support nucleic acid synthesis in cancers and provides the first metabolic link between the urea cycle enzymes and pyrimidine synthesis. In addition, the results described herein show for the first time that citrin is essential for cancer cells' proliferation as a transporter of mitochondrial aspartate for pyrimidine synthesis and that knockdown of citrin decreases cancer cells' survival in ASS1 deficient cancers. Moreover, the present inventors show that cancer patients with citrin overexpression and ASS1 deficiency have a worse prognosis. In addition, the present inventors further show that citrin deficient fibroblasts have decreased glycolysis and decreased ATP and NADH production (FIGS. 9A-C) and may have increased autophagy (FIGS. 10A-B). Moreover, the present inventors show that silencing Citrin in cancer decreases cells' proliferation (FIGS. 11A-C), and that citrin is over expressed specifically in hepatocytes and cancer cells (FIG. 12). Indeed, the present inventors further show that citrin over expressing cancer cells have increased glycolysis (FIGS. 13A-B) and enlarged mitochondrias (FIGS. 14A-D), suggesting enhanced energy production. These results show that citrin is over expressed in cancer as it is required for cancer cells' energy production by enhancing glycolysis and NADH synthesis, as well as for transporting aspartate. When Citrin is deficient, cancer cells show decreased proliferation and go through autophgocytosis. Thus, targeting citrin as cancer therapy could be beneficial for treating cancers.

According to an aspect of some embodiments of the invention, there is provided a method of treating cancer in a subject in need thereof, the method comprising administering to the subject a therapeutically effective amount of an agent which downregulates a mitochondrial aspartate-dependent pyrimidine synthesis, thereby treating the cancer.

According to an aspect of some embodiments of the invention there is provided an agent which down-regulates a mitochondrial aspartate-dependent pyrimidine synthesis for treating cancer in a subject in need thereof.

The term “treating” refers to inhibiting, preventing or arresting the development of a pathology (cancer) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology. Preferably, this term encompasses individuals who are at risk to develop the pathology.

The cancer which can be treated by the method of some embodiments of the invention and/or by the pharmaceutical composition of some embodiments of the invention can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibro sarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to some embodiments of the invention, the cancer is characterized by an increased level of citrin as compared to the level of the citrin in a non-malignant tissue of the same origin as the cancer.

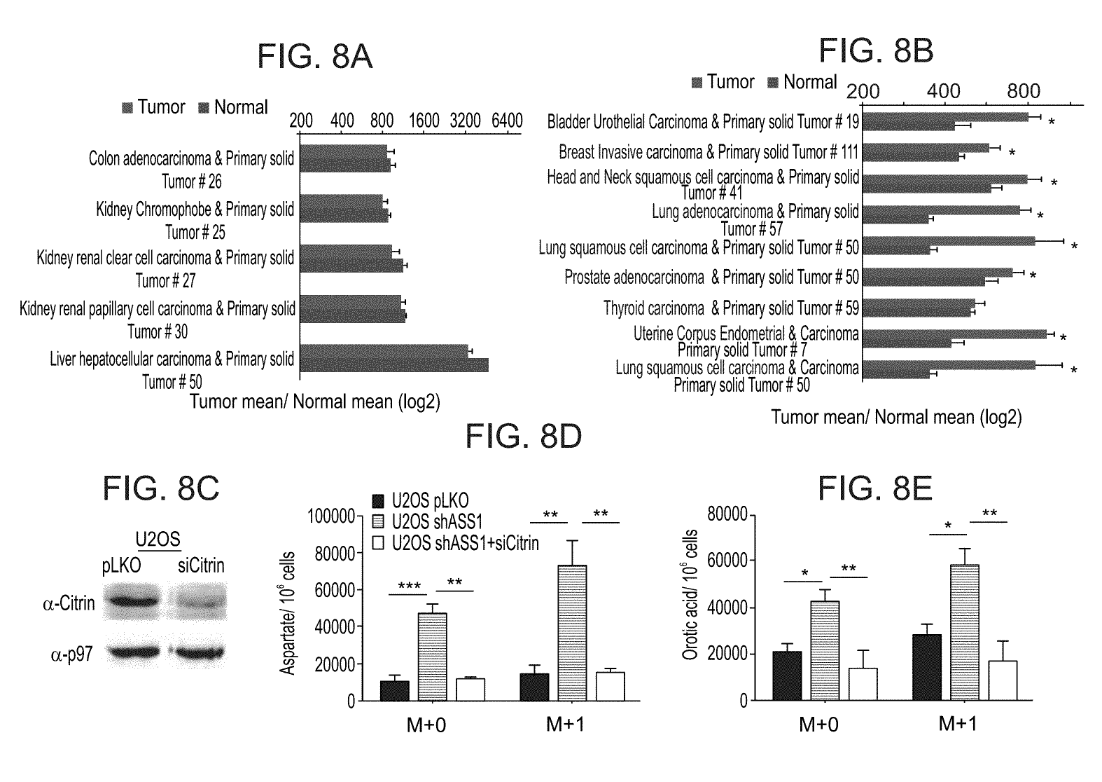

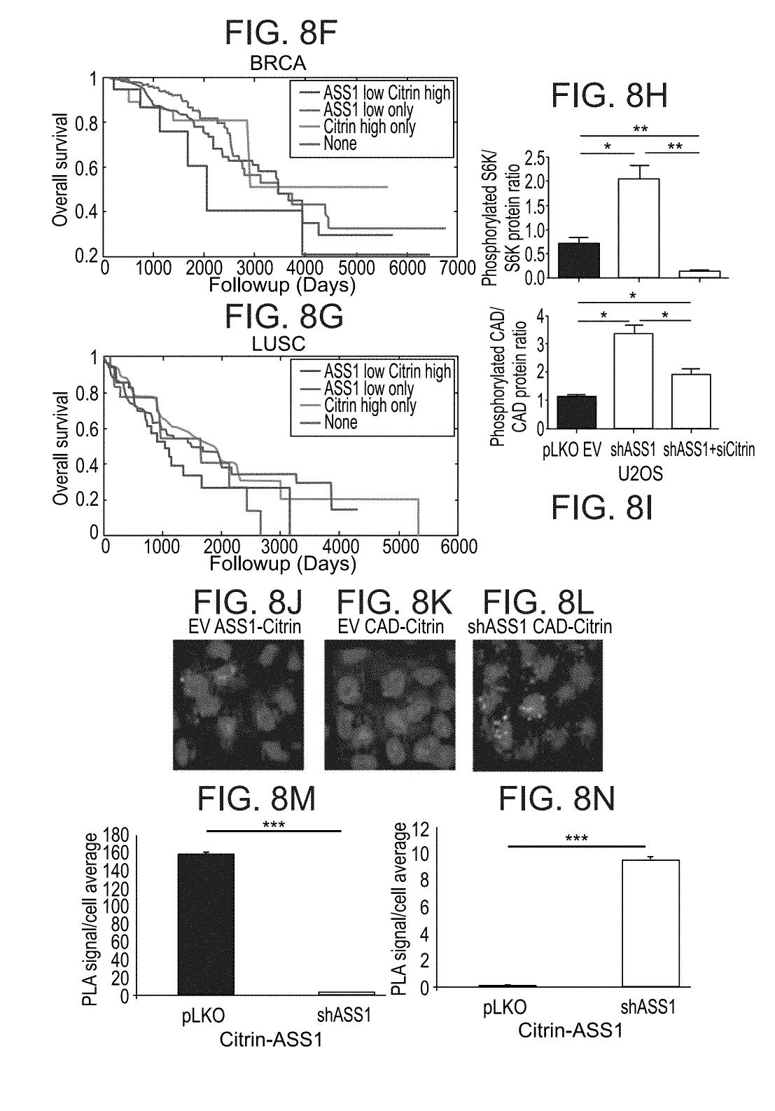

For example, the level of citrin in the cancer can be at least 10%, at least 20%, at least 30%, at least 40%, at least 50%, at least 60%, at least 70%, at least 80%, at least 90%, e.g., 100%, at least 200%, at least 300%, at least 400%, at least 500%, at least 600% higher than the level of the citrin in a non-malignant tissue of the same origin as the cancer as measured under identical assay conditions, using e.g., any RNA and or protein detection method suitable for measuring citrin levels (described hereinbelow). A non-limiting example is given in FIG. 8C in which the level of citrin was measured by Western blot analysis.

According to some embodiments of the invention, the cancer is characterized by a decreased level of Argininosuccinate synthase as compared to the level of the Argininosuccinate synthase in a non-malignant tissue of the same origin as the cancer.

For example, the level of Argininosuccinate synthase in the cancer can be at least 10%, at least 20%, at least 30%, at least 40%, at least 50%, at least 60%, at least 70%, at least 80%, at least 90%, e.g., 100% lower as compared to the level of the Argininosuccinate synthase in a non-malignant tissue of the same origin as the cancer as measured under identical assay conditions, using e.g., any RNA and or protein detection method suitable for measuring ASS1 levels (described hereinbelow). A non-limiting example is given in FIG. 2E and FIGS. 3A-B in which the level of ASS1 was measured by Western blot analysis.

According to some embodiments of the invention, the cancer is a liver cancer, a bladder cancer, a breast cancer (e.g., breast invasive carcinoma and primary solid tumor), a head and neck squamous cell carcinoma, a lung adenocarcinoma, a lung squamous cell carcinoma, a prostate adenocarcinoma, and a uterine corpus endometrial.

According to some embodiments of the invention, the cancer is a liver cancer. As used herein the phrase “a mitochondrial aspartate-dependent pyrimidine synthesis” refers to the metabolic pathway by which mitochondrial aspartate is transported to the cytosol and used for pyrimidine synthesis. As mentioned in the background section, the metabolic pathway by which mitochondrial aspartate is transported to the cytosol and used for pyrimidine synthesis comprises citrin.

The term “citrin” or “solute carrier family 25 member 13 (SLC25A13)” or “Aralar2”, which are interchangeably used herein, refers to a protein member of the mitochondrial carrier family which catalyzes the exchange of aspartate for glutamate and a proton across the inner mitochondrial membrane, and is stimulated by calcium on the external side of the inner mitochondrial membrane. The citrin protein contains four EF-hand Ca(2+) binding motifs in the N-terminal domain, and is localized in the mitochondria. Two citrin protein variants are known as “calcium-binding mitochondrial carrier protein Aralar2 isoform 1” [GenBank Accession No. NP_001153682.1 (SEQ ID NO:1) encoded by the sequence depicted in GenBank Accession No. NM_001160210.1 (SEQ ID NO: 2)] and calcium-binding mitochondrial carrier protein Aralar2 isoform 2 [GenBank Accession No. NP_055066.1 (SEQ ID NO:3) encoded by the sequence depicted in GenBank Accession No. NM_014251.2 (SEQ ID NO: 4)].

As mentioned in the background section, the metabolic pathway by which mitochondrial aspartate is transported to the cytosol and used for pyrimidine synthesis comprises CAD.

As used herein the term “CAD” refers to the trifunctional protein which is associated with the enzymatic activities of the first 3 enzymes in the 6-step pathway of pyrimidine biosynthesis: carbamoylphosphate synthetase (CPS II), aspartate transcarbamoylase, and dihydroorotase. The trifunctional protein encoded by CAD is regulated by the mitogen-activated protein kinase (MAPK) cascade, which indicates a direct link between activation of the MAPK cascade and de novo biosynthesis of pyrimidine nucleotides.

The CAD protein is encoded by the CAD gene (Gene ID: 790). The coding sequences of the CAD protein are known in the art and can be obtained from various sources. The sequence depicted in GenBank Accession No. NM_004341.4 (SEQ ID NO: 29) encodes the CAD protein isoform 1 (GenBank Accession No. NP_004332.2, SEQ ID NO: 28) which represents the longer isoform. The sequence depicted in GenBank Accession No. NM_001306079.1 (SEQ ID NO: 31) encodes the CAD protein isoform 2 (GenBank Accession No. NP_001293008.1, SEQ ID NO: 30) which represents a variant (2) which lacks an alternate in-frame exon in the 5′ coding region compared to variant 1, and accordingly is shorter than isoform 1.

Carbamoyl phosphate synthetase (also known as Carbamoyl-phosphate synthase (glutamine-hydrolyzing); Carbamoyl-phosphate synthetase 2 enzyme; ENZYME entry: EC 6.3.5.5) catalyzes the reaction of 2 ATP+L-glutamine+HCO(3)(−)+H(2)O⇔2 ADP+phosphate+L-glutamate+carbamoyl phosphate.

Aspartate transcarbamylase (also known as Aspartate carbamoyltransferase; ENZYME entry: EC 2.1.3.2) catalyzes the reaction of Carbamoyl phosphate+L-aspartate ⇔phosphate+N-carbamoyl-L-aspartate.

Dihydroorotase (also known as Carbamoylaspartic dehydrase; ENZYME entry: EC 3.5.2.3) catalyses the reaction of (S)-dihydroorotate+H(2)O⇔N-carbamoyl-L-aspartate.

According to some embodiments of the invention, the agent which downregulates a mitochondrial aspartate-dependent pyrimidine synthesis is an agent which downregulates activity and/or expression of a polypeptide or an enzyme selected from the group consisting of citrin, carbamoyl-phosphate synthase 2, aspartate transcarbamylase and dihydroorotase.

According to some embodiments of the invention the agent which downregulates a mitochondrial aspartate-dependent pyrimidine synthesis is an agent which downregulates the expression and/or the activity of citrin and/or the expression and/or the activity of CAD.

According to some embodiments of the invention, the agent which downregulates a mitochondrial aspartate-dependent pyrimidine synthesis is an agent which downregulates activity and/or expression of citrin.

According to some embodiments of the invention, the agent which downregulates a mitochondrial aspartate-dependent pyrimidine synthesis comprises a plurality of agents for downregulating activity and/or expression of at least two of the citrin, carbamoyl-phosphate synthase 2, aspartate transcarbamylase and dihydroorotase.

According to some embodiments of the invention, the agent which downregulates a mitochondrial aspartate-dependent pyrimidine synthesis comprises a plurality of agents for downregulating activity and/or expression of at least three of the citrin, carbamoyl-phosphate synthase 2, aspartate transcarbamylase and dihydroorotase.

According to some embodiments of the invention, the agent which downregulates a mitochondrial aspartate-dependent pyrimidine synthesis comprises a plurality of agents for downregulating activity and/or expression of the citrin, carbamoyl-phosphate synthase 2, aspartate transcarbamylase and dihydroorotase.

As used herein the phrase “downregulates expression” refers to downregulating the expression of a protein (e.g. a protein involved in the mitochondrial aspartate-dependent pyrimidine synthesis such as citrin or CAD) at the genomic (e.g., homologous recombination, genome editing and/or site specific endonucleases) and/or the transcript level using a variety of molecules which interfere with transcription and/or translation [e.g., RNA silencing agents (e.g., antisense, siRNA, shRNA, micro-RNA), Ribozyme, DNAzyme, TFO] or on the protein level (e.g., aptamers, small molecules and inhibitory peptides, antagonists, enzymes that cleave the polypeptide, antibodies and the like).

For the same culture conditions the expression is generally expressed in comparison to the expression in a cell of the same species but not contacted with the agent or contacted with a vehicle control, also referred to as control.

Down regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down-regulating the mitochondrial aspartate-dependent pyrimidine synthesis e.g., via downregulation of citrin and/or CAD expression are described in details hereinbelow.

Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, downregulation of the mitochondrial aspartate-dependent pyrimidine synthesis (e.g., of citrin and/or CAD) can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., citrin and/or CAD) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005, Nucleic Acids Res. 33:4140-56). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ (SEQ ID NO:32) and 5′-UUACAA-3′ (SEQ ID NO:33) (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the citrin mRNA sequence (SEQ ID NO:2 or 4) or the CAD mRNA sequence (SEQ ID NO:29 or 31) is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl 2001, ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server(www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, suitable siRNAs directed against citrin can be the sh901-5′-TTAAGAAAGTGCTACGCTA-3′ (SEQ ID NO:26); and/or the sh909-5′-GTATCCTATCGATCTTGTA-3′ (SEQ ID NO:27); and/or the sh917-5′-TGGGAGAACTCATGTATAA-3′ (SEQ ID NO: 45).

Additionally or alternatively, a citrin siRNA can be obtained from Santa Cruz Biotechnology (Dallas. Tex., U.S.A.) e.g., Catalogue No. sc-89601.

It should be noted that the effect of the siRNA on the level of citrin in the cell can be detected by an anti-citrin antibody such as the D-7 clone (Santa Cruz Biotechnology Catalogue No. sc-393303) which is directed at an epitope mapping between amino acids 30-51 near the N-terminus of human citrin.

An exemplary siRNA against CAD mRNA (SEQ ID NO: 29, GenBank Accession No. NM_004341.4) is the Trilencer-27 Human siRNA available from ORIGENE Catalogue number SR300555 (OriGene Technologies, Inc. Rockville USA, Catalogue No. KN209469).

It will be appreciated that the RNA silencing agent of some embodiments of the invention needs not be limited to those molecules containing only RNA, but can further encompass chemically-modified nucleotides and non-nucleotides.

miRNA and miRNA mimics—According to another embodiment the RNA silencing agent may be a miRNA.

The term “microRNA”, “micro-RNA”, “miRNA”, and “miR” are synonymous and refer to a collection of non-coding single-stranded RNA molecules of about 19-28 nucleotides in length, which regulate gene expression. miRNAs are found in a wide range of organisms (viruses, fwdarw, humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression.

miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O, 4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. For example, in order to target and downregulate CAD, the sequence of the miRNA may comprise the sequence miR-224-5p- for CAD [MIMAT0000281: 3′-UUGCCUUGGUGAUCACUGAAC-5′ (SEQ ID NO: 34)].

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of citrin and/or CAD can be effected using an antisense polynucleotide capable of specifically hybridizing with an mRNA transcript encoding citrin and/or CAD.

Design of antisense molecules which can be used to efficiently downregulate a citrin and/or CAD must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

For example, suitable antisense oligonucleotides targeted against the citrin and/or CAD protein mRNA (which is coding for the citrin and/or CAD protein, respectively) would be of the following sequences:

Exemplary antisense against the citrin RNA include: (1). 5′-UUAAGAAAGUGCUACGCUA-3′ (SEQ ID NO:35), available from Dharmacon (GE Dharmacon Lafayette, Colo., USA) Catalog number: D-007472-17; (2) 5′-CAAGUUAGUUUCUCCUAUU-3′ (SEQ ID NO:36), available from Dharmacon Catalog number: D-007472-04; (3) 5′-GAUCAGAGCCAGUUCCUAA-3′ (SEQ ID NO:37, available from Dharmacon Catalog number: D-007472-03; (4). 5′-GACCAAAGAUGGAUUAAUA-3′ (SEQ ID NO:38), available from Dharmacon Catalog number: D-007472-01.

An exemplary antisense against the CAD RNA is the 5′-TTCACCACACAATAATCCACG-3′ (SEQ ID NO: 39) available from Dharmacon Catalog number: RHS3979-200800098.

Nucleic acid agents can also operate at the DNA level as summarized infra.

In some embodiments, the RNA silencing agent provided herein can be functionally associated with a “cell-penetrating peptide”. As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell and/or mitochondrial membrane. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIsl, TAT(48-60), pVEC, MTS, and MAP.

Downregulation of the mitochondrial aspartate-dependent pyrimidine synthesis e.g., via downregulation of citrin and/or CAD protein(s) can also be achieved by inactivating the gene encoding the proteins (e.g., citrin and/or CAD protein) via introducing targeted mutations involving loss-of function alterations (e.g. point mutations, deletions and insertions) in the gene structure.

As used herein, the phrase “loss-of-function alterations” refers to any mutation in the DNA sequence of a gene encoding the citrin protein (set forth by SEQ ID NO: 1 or 3) and/or a gene encoding the CAD protein (set forth by SEQ ID NO: 28 or 30) which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the citrin and/or CAD mutation may be in a homozygous form or in a heterozygous form. According to this embodiment, homozygosity is a condition where both alleles at the e.g. citrin and/or CAD locus are characterized by the same nucleotide sequence. Heterozygosity refers to different conditions of the gene at the e.g. citrin and/or CAD locus.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www://www.talendesign.org). TALEN can also be designed and obtained commercially from e.g., Sangamo Biosciences™ (Richmond, Calif.).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

Non-limiting examples of gRNAs that can be used in the methods of some embodiments of the present invention include 5′-GCCATGATTCGCCCCGGTTG-3′ (SEQ ID NO: 40) which corresponds to exon 1 of the citrin gene; and 5′-TGACCTTAGCAGACATTGAA-3′ (SEQ ID NO: 41) which corresponds to exon 9 of the citrin gene.

A non-limiting example for a gene knockout kit via CRISPR of the human CAD protein is available from ORIGENE Catalogue No. KN209469 (OriGene Technologies, Inc. Rockville USA).

The CRISPR/cas system was usefully used for genome editing of a mitochondrial protein as described elsewhere [Areum Jo et al., 2015. “Efficient Mitochondrial Genome Editing by CRISPR/Cas9”, BioMed Research International Volume 2015, Article ID 305716; which is fully incorporated herein by reference in its entirety]. Briefly, sgRNAs targeting specific loci of the mitochondrial genome along with a mitochondria-targeted Cas9 (mitoCas9) are generated essentially as described in Areum Jo et al., 2015 (supra).

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRY”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas To12 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the mitochondrial aspartate-dependent pyrimidine synthesis e.g., by downregulation of the expression level and/or activity of citrin and/or CAD may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosyltransferase (ARPT).

Ribozymes

Another agent capable of downregulating the mitochondrial aspartate-dependent pyrimidine synthesis e.g., by downregulating citrin and/or CAD is a ribozyme molecule capable of specifically cleaving an mRNA transcript encoding citrin and/or CAD. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

DNAzymes

Another agent capable of downregulating the mitochondrial aspartate-dependent pyrimidine synthesis e.g., by downregulating citrin and/or CAD is a DNAzyme molecule capable of specifically cleaving an mRNA transcript or DNA sequence of the citrin and/or CAD. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 2002, Abstract 409, Ann Meeting Am Soc Gen Ther (www(dot)asgt(dot)org)). In another application, DNAzymes complementary to bcr-ab1 oncogenes were successful in inhibiting the oncogenes expression in leukemia cells, and lessening relapse rates in autologous bone marrow transplant in cases of CML and ALL.

An additional method of down regulating the mitochondrial aspartate-dependent pyrimidine synthesis e.g., by downregulating the expression of the gene encoding citrin and/or the gene encoding CAD in cells is via triplex forming oligonucleotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

oligo 3′--A G G T
duplex 5′--A G C T
duplex 3′--T C G A

However, it has been shown that the A-AT and G-GC triplets have the greatest triple helical stability (Reither and Jeltsch, BMC Biochem, 2002, September12, Epub). The same authors have demonstrated that TFOs designed according to the A-AT and G-GC rule do not form non-specific triplexes, indicating that the triplex formation is indeed sequence specific.

Thus for any given sequence in the citrin regulatory region and/or the CAD regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

According to some embodiments of the invention, the agent which downregulates a mitochondrial aspartate-dependent pyrimidine synthesis is an oligonucleotide.

According to some embodiments of the invention, the oligonucleotide is selected from the group consisting of an RNA silencing agent and a genome editing agent.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions.

Oligonucleotides designed according to the teachings of some embodiments of the invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of some embodiments of the invention is of at least 17, at least 18, at least 19, at least 20, at least 22, at least 25, at least 30 or at least 40, bases specifically hybridizable with sequence alterations described hereinabove.

The oligonucleotides of some embodiments of the invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder.

Specific examples of preferred oligonucleotides useful according to some embodiments of the invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to some embodiments of the invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in some embodiments of the invention are disclosed in U.S. Pat. No. 6,303,374.

Oligonucleotides of some embodiments of the invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications. Still further base substitutions include the non-standard bases disclosed in U.S. Pat. Nos. 8,586,303, 8,614,072, 8,871,469 and 9,062,336, all to Benner et al: for example, the non-standard dZ:dP nucleobase pair which Benner et al has shown can be incorporated into DNA by DNA polymerases to yield amplicons with multiple non-standard nucleotides.

Down-Regulation at the Polypeptide Level

Aptamers

Another agent which can downregulate the mitochondrial aspartate-dependent pyrimidine synthesis (e.g., by downregulating citrin and/or CAD) is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another agent which can downregulate the mitochondrial aspartate-dependent pyrimidine synthesis would be any molecule which binds to and/or cleaves proteins in the mitochondrial aspartate-dependent pyrimidine synthesis pathway such as citrin and/or CAD. Such molecules can be a small molecule, an antagonist, an inhibitory peptide, and/or an enzyme that cleaves the proteins in the mitochondrial aspartate-dependent pyrimidine synthesis pathway such as citrin and/or CAD.

It will be appreciated that a non-functional analogue of at least a catalytic or binding portion of citrin and/or CAD can be also used as an agent which downregulates citrin and/or CAD.

Alternatively or additionally, small molecule or peptides can be used which interfere with citrin and/or CAD protein function (e.g., catalytic or interaction).

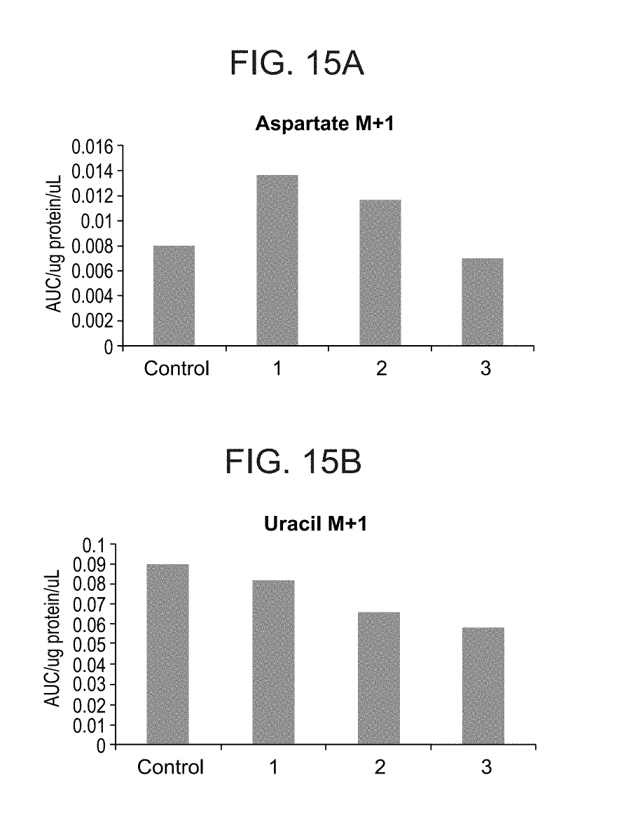

For example, as shown in Example 5 of the Examples section which follows, the present inventors have demonstrated that small molecules such as compound “3” (L-Glutamic acid γ-benzyl ester) inhibited citrin activity as is evidenced by the decrease in the level of aspartate M+1 (FIG. 15A) and of uracil M+1 (FIG. 15B). In addition, compounds “1” (D-Glutamic acid) and “2” (L-glutamic acid-5-methyl ester) also inhibited Uracil M+1 thus indicating their ability to inhibit citrin (FIG. 15B).

According to some embodiments of the invention, the agent comprises L-Glutamic acid γ-benzyl ester.

According to some embodiments of the invention, the agent is L-Glutamic acid γ-benzyl ester.

According to some embodiments of the invention, the agent is L-glutamic acid-5-methyl ester.

According to some embodiments of the invention, the agent is D-Glutamic acid.

According to some embodiments of the invention, the agent is selected from the group consisting of L-Glutamic acid γ-benzyl ester, L-glutamic acid-5-methyl ester and D-Glutamic acid.

Known inhibitors of citrin (based on in vitro studies) which can be used for treating cancer according to some embodiments of the invention include pyridoxal 5″-phosphate (e.g., at about 10 mM; The EMBO journal. Vol 20 No. 18 pp. 5060-5069, 2001), bathophenanthroline (e.g., at about 10 mM), Mercurials [HgCl₂, mersalyl and p-chloromercuriphenylsulfonate (e.g., at about 10 μM)], mersalyl (e.g., at about 10 μM), p-chloromercuriphenylsulfonate (e.g., at about 10 μM), diethyl pyrocarbonate (e.g., at about 1 mM), N-ethylmaleimid (e.g., at 1 about mM, known to inhibit about 50% of citrin in vitro), 1,2,3-benzenetricarboxylate [Table 2 in: BBA-Molecular cell research (2016). Doi: 10.1016/j.bbamcr.2016.04.011], Carboxyatractyloside, α-cyano-4-hydroxycinnamate, or any combinations thereof. It should be noted that the above concentrations of citrin inhibitors can be adjusted for in vivo administration using animal models for the specific cancers (e.g., with xenografts of human tumors).

Another agent which can be used along with some embodiments of the invention to downregulate the mitochondrial aspartate-dependent pyrimidine synthesis is a molecule which prevents activation or substrate binding of at least one protein of the mitochondrial aspartate-dependent pyrimidine synthesis pathway such as citrin and/or CAD.

According to some embodiments of the invention, the agent which downregulates a mitochondrial aspartate-dependent pyrimidine synthesis is a small molecule.

Antibodies

According to specific embodiments the agent capable of downregulating the mitochondrial aspartate-dependent pyrimidine synthesis is an antibody or antibody fragment capable of specifically binding to a protein of the mitochondrial aspartate-dependent pyrimidine synthesis pathway such as citrin and/or CAD.

As used herein, the term “antibody” refers to a substantially intact antibody molecule.

As used herein, the phrase “antibody fragment” refers to a functional fragment of an antibody (such as Fab, F(ab′)2, Fv or single domain molecules such as VH and VL) that is capable of binding to an epitope of an antigen.

Suitable Antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv, an Fab, an Fab′, and an F(ab′)2.

Functional antibody fragments comprising whole or essentially whole variable regions of both light and heavy chains are defined as follows:

(i) Fv, defined as a genetically engineered fragment consisting of the variable region of the light chain and the variable region of the heavy chain expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(iv) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(v) F(ab′) 2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vi) Single domain antibodies are composed of a single VH or VL domains which exhibit sufficient affinity to the antigen.

Preferably, the antibody specifically binds at least one epitope of a citrin and/or CAD. As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

Methods of generating antibodies (i.e., monoclonal and polyclonal) are well known in the art (See for example, Harlow and Lane, Antibodies: A Laboratory Manual, Cold Spring Harbor Laboratory, New York, 1988, incorporated herein by reference). Antibodies may be generated via any one of several methods known in the art, which methods can employ induction of in-vivo production of antibody molecules, screening of immunoglobulin libraries (Orlandi D. R. et al., 1989. Proc. Natl. Acad. Sci. U.S.A 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote R J. et al., 1983. Proc. Natl. Acad. Sci. U.S.A 80:2026-2030; Cole S P. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumine (BSA)] carriers (see, for example, U.S. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments can be obtained using methods well known in the art. [see, for example, Harlow and Lane, “Antibodies: A Laboratory Manual”, Cold Spring Harbor Laboratory, New York, (1988)]. For example, antibody fragments according to some embodiments of the invention can be prepared by proteolytic hydrolysis of the antibody or by expression in E. coli or mammalian cells (e.g., Chinese hamster ovary cell culture or other protein expression systems) of DNA encoding the fragment.

Alternatively, antibody fragments can be obtained by pepsin or papain digestion of whole antibodies by conventional methods. As described hereinabove, an (Fab′)2 antibody fragments can be produced by enzymatic cleavage of antibodies with pepsin to provide a 5S fragment. This fragment can be further cleaved using a thiol reducing agent, and optionally a blocking group for the sulfhydryl groups resulting from cleavage of disulfide linkages to produce 3.5S Fab′ monovalent fragments. Alternatively, enzymatic cleavage using pepsin produces two monovalent Fab′ fragments and an Fc fragment directly. Ample guidance for practicing such methods is provided in the literature of the art (for example, refer to: Goldenberg, U.S. Pat. Nos. 4,036,945 and 4,331,647; Porter, R R., 1959. Biochem. J. 73:119-126). Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, an Fv is composed of paired heavy chain variable and light chain variable domains. This association may be noncovalent (see, for example, Inbar et al., 1972. Proc. Natl. Acad. Sci. USA. 69:2659-62). Alternatively, as described hereinabove the variable domains can be linked to generate a single chain Fv by an intermolecular disulfide bond, or alternately, such chains may be cross-linked by chemicals such as glutaraldehyde.

Preferably, the Fv is a single chain Fv.

Single chain Fv's are prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains. Ample guidance for producing single chain Fv's is provided in the literature of the art (for example, refer to: Whitlow and Filpula, 1991. Methods 2:97-105; Bird et al., 1988. Science 242:423-426; Pack et al., 1993. Bio/Technology 11:1271-77; and Ladner et al., U.S. Pat. No. 4,946,778).

Isolated complementarity determining region peptides can be obtained by constructing genes encoding the complementarity determining region of an antibody of interest. Such genes may be prepared, for example, by RT-PCR of mRNA of an antibody-producing cell. Ample guidance for practicing such methods is provided in the literature of the art (for example, refer to Larrick and Fry, 1991. Methods 2:106-10).

It will be appreciated that for human therapy or diagnostics, humanized antibodies are preferably used. Humanized forms of non human (e.g., murine) antibodies are genetically engineered chimeric antibodies or antibody fragments having—preferably minimal—portions derived from non human antibodies. Humanized antibodies include antibodies in which complementary determining regions of a human antibody (recipient antibody) are replaced by residues from a complementarity determining region of a non human species (donor antibody) such as mouse, rat or rabbit having the desired functionality. In some instances, Fv framework residues of the human antibody are replaced by corresponding non human residues. Humanized antibodies may also comprise residues which are found neither in the recipient antibody nor in the imported complementarity determining region or framework sequences. In general, the humanized antibody will comprise substantially all of at least one, and typically two, variable domains, in which all or substantially all of the complementarity determining regions correspond to those of a non human antibody and all, or substantially all, of the framework regions correspond to those of a relevant human consensus sequence. Humanized antibodies optimally also include at least a portion of an antibody constant region, such as an Fc region, typically derived from a human antibody (see, for example, Jones et al., 1986. Nature 321:522-525; Riechmann et al., 1988. Nature 332:323-329; and Presta, 1992. Curr. Op. Struct. Biol. 2:593-596).

Methods for humanizing non human antibodies are well known in the art. Generally, a humanized antibody has one or more amino acid residues introduced into it from a source which is non human. These non human amino acid residues are often referred to as imported residues which are typically taken from an imported variable domain. Humanization can be essentially performed as described (see, for example: Jones et al., 1986. Nature 321:522-525; Riechmann et al., 1988. Nature 332:323-327; Verhoeyen et al., 1988. Science 239:1534-1536; U.S. Pat. No. 4,816,567) by substituting human complementarity determining regions with corresponding rodent complementarity determining regions. Accordingly, such humanized antibodies are chimeric antibodies, wherein substantially less than an intact human variable domain has been substituted by the corresponding sequence from a non human species. In practice, humanized antibodies may be typically human antibodies in which some complementarity determining region residues and possibly some framework residues are substituted by residues from analogous sites in rodent antibodies.

Human antibodies can also be produced using various techniques known in the art, including phage display libraries [see, for example, Hoogenboom and Winter, 1991. J. Mol. Biol. 227:381; Marks et al., 1991. J. Mol. Biol. 222:581; Cole et al., “Monoclonal Antibodies and Cancer Therapy”, Alan R. Liss, pp. 77 (1985); Boerner et al., 1991. J. Immunol. 147:86-95). Humanized antibodies can also be made by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807, 5,545,806, 5,569,825, 5,625,126, 5,633,425, and 5,661,016; Marks et al., 1992. Bio/Technology 10:779-783; Lonberg et al., 1994. Nature 368:856-859; Morrison, 1994. Nature 368:812-13; Fishwild et al., 1996. Nature Biotechnology 14:845-51; Neuberger, 1996. Nature Biotechnology 14:826; Lonberg and Huszar, 1995. Intern. Rev. Immunol. 13:65-93).

As citrin is localized in the inner membrane of the mitochondria an antibody or antibody fragment capable of specifically binding citrin is typically an intracellular antibody.

Similarly, as CAD is localized intracellularly (e.g., within the cytosol), an antibody or antibody fragment capable of specifically binding CAD is typically an intracellular antibody.

Intracellular antibodies (also known as “intrabodies”) are essentially single chain antibodies to which intracellular localization signals have been added (e.g., ER, mitochondrial, nuclear, cytoplasmic). This technology has been successfully applied in the art (for review, see Richardson and Marasco, 1995, TIBTECH vol. 13). Intrabodies have been shown to virtually eliminate the expression of otherwise abundant cell surface receptors and to inhibit a protein function within a cell (See, for example, Richardson et al., 1995, Proc. Natl. Acad. Sci. USA 92: 3137-3141; Deshane et al., 1994, Gene Ther. 1: 332-337; Marasco et al., 1998 Human Gene Ther 9: 1627-42; Shaheen et al., 1996 J. Virol. 70: 3392-400; Werge, T. M. et al., 1990, FEBS Letters 274:193-198; Carlson, J. R. 1993 Proc. Natl. Acad. Sci. USA 90:7427-7428; Biocca, S. et al., 1994, Bio/Technology 12: 396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J. Biol. Chem. 269:23931-23936; Mhashilkar, A. M. et al., 1995, EMBO J. 14:1542-1551; PCT Publication No. WO 94/02610 by Marasco et al.; and PCT Publication No. WO 95/03832 by Duan et al.).

To prepare an intracellular antibody expression vector, the cDNA encoding the antibody light and heavy chains specific for the target protein of interest are isolated, typically from a hybridoma that secretes a monoclonal antibody specific for the marker. Hybridomas secreting anti-marker monoclonal antibodies, or recombinant monoclonal antibodies, can be prepared using methods known in the art. Once a monoclonal antibody specific for the marker protein is identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process and the nucleotide sequences of antibody light and heavy chain genes are determined. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database. Once obtained, the antibody light and heavy chain sequences are cloned into a recombinant expression vector using standard methods.

For cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In another embodiment, the vector encodes a single chain antibody (scFv) wherein the variable regions of the light and heavy chains are linked by a flexible peptide linker [e.g., (Gly₄Ser)₃ and expressed as a single chain molecule. To inhibit marker activity in a cell, the expression vector encoding the intracellular antibody is introduced into the cell by standard transfection methods, as discussed hereinbefore.

Intrabodies for expression in the mitochondria were taught in Lo A S., et al. 2008; “Intracellular antibodies (intrabodies) and their therapeutic potential”, Handb Exp Pharmacol. 181:343-73; and Boldicke T, 2007. “Blocking translocation of cell surface molecules from the ER to the cell surface by intracellular antibodies targeted to the ER.”, J. Cell Mol. Med. 11(1): 54-70; each of which is fully incorporated herein by reference.

Once antibodies are obtained, they may be tested for activity, for example via ELISA (enzyme-linked immunosorbent assay).

It should be noted that the antibodies of some embodiments of the invention can be conjugated to a functional moiety such as a detectable or a therapeutic moiety (also referred to as an “immunoconjugate”). The immunoconjugate molecule can be an isolated molecule such as a soluble and/or a synthetic molecule.

The therapeutic moieties can be any type of toxin such as Pseudomonas exotoxin, Diphtheria toxin, Ricin toxin, and PE38KDEL.

When the functional moiety is a polypeptide, the immunoconjugate may be produced by recombinant means, e.g., by ligating the coding sequence of the functional moiety in-frame with the nucleic acid sequence encoding the antibody of the invention and expressing in a host cell to produce a recombinant conjugated antibody. Alternatively, the functional moiety may be chemically synthesized by, for example, the stepwise addition of one or more amino acid residues in defined order such as solid phase peptide synthetic techniques.

A functional moiety may also be attached to the antibody of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

According to some embodiments of the invention the method further comprising administering to the subject an arginine depletion therapy as a combination therapy with the agent(s) which downregulate the mitochondrial aspartate-dependent pyrimidine synthesis.

Non-limiting examples of an agent which can deplete arginine in a tumor cell include the arginine catabolizing enzyme such as arginine deiminase (ADI) and arginase I.

ADI-PEG 20 is a formulation of ADI with polyethylene glycol (PEG) having an average molecular weight of 20 kilodaltons (PEG 20).

Arginase (Gene ID: 383, gene symbol: ARG1) catalyzes the hydrolysis of arginine to ornithine and urea. A pegylated form of the catabolic enzyme arginase I (peg-Arg I) can be used [Fletcher M et al., 2015. “1-Arginine depletion blunts antitumor T-cell responses by inducing myeloid-derived suppressor cells”, Cancer Res. 2015 Jan. 15; 75(2):275-83, which is fully incorporated herein by reference].

According to some embodiments of the invention the method further comprising administering to the subject an agent for glutamine depletion.

According to some embodiments of the invention, the agent for glutamine depletion acts on intracellular and/or extracellular glutamine, e.g., on the glutamine present in the cytosol and/or the mitochondria, and/or on the glutamine present in the peripheral blood.

According to some embodiments of the invention, the agent for glutamine depletion acts on the glutamine present in the cytosol.

According to some embodiments of the invention, the agent for glutamine depletion acts on the glutamine present in the blood.

According to some embodiments of the invention, the agent for glutamine depletion acts on the mitochondria and/or the cytosol.

Examples of agents which can deplete glutamine in tumor cells include, but are not limited to, inhibitors of glutamate-oxaloacetate-transaminase (GOT).

Aminooxyacetate (AOA), an inhibitor of glutamate-dependent transaminase, has also been shown to target glutamine metabolism and to sensitize melanoma cells to TRAIL-induced death (Qin J Z., et al., 2010. Biochem. Biophys. Res. Commun. 398: 146-52). Mahesh Saqcena, et al., 2015 [Mahesh Saqcena, et al., 2015, “Apoptotic effects of high-dose rapamycin occur in S-phase of the cell cycle”, Cell Cycle. 14(14): 2285-2292, which is fully incorporate herein by reference] teaches blocking the anaplerotic utilization of Gln with aminooxyacetate (AOA), which interferes with the transamination reaction whereby glutamate is deaminated to a-ketoglutarate while oxaloacetate is aminated to aspartic acid by the enzyme glutamate-oxaloacetate-transaminase (GOT).

According to some embodiments of the invention the method further comprising administering to the subject chemotherapy which inhibits production of nucleotides.

Various chemotherapy agents are known in the art and are used for treating cancer. These include pyrimidines analogues, purine analogs and folate antagonists.

Pyrimidine analogs which can be used according to the method of some embodiments of the invention include, but are not limited to arabinosylcytosine, gemcitabine and decitabine. Arabinosylcytosine or cytarabine is a deooxycytidine base compound that is converted to its active metabolite, ara-CTP. This base is incorporated into DNA and causes strand termination. Decitabine, is phosphorylated and directly incorporated into DNA. In cancer cells, it stops methylation by inhibiting DNA methytransferase and induces cell death.

Purine analogs which can be used according to the method of some embodiments of the invention include, but are not limited to Fludarabine or 2-fluoro-ara-amp (an antimetabolite of the purine class), which functions as a pro-drug and 6-Mercaptopurine (6-MP). Fludarabine is dephosphorylated and enters the cancer cell and then it is rephosphorylated to F-ara-ATP. Upon incorporation into the DNA strand, F-ara-ATP halts strand lengthening. 6-Mercaptopurine (6-MP) is another purine agent successfully used against acute lymphocytic leukemia. 6-MP is active in the S phase of cell proliferation and upon incorporation into DNA and RNA, the nucleic acids are rendered useless.

Folate antagonist which can be used according to the method of some embodiments of the invention include, but are not limited to methotrexate, and pemetrexed. Methotrexate, binds to the enzyme DI-IFR reversibly and inactivates it, thus, preventing methylation and decreasing available supplies of purine and thymidine bases for new DNA and RNA synthesis. Methotrexate is active in the S phase of cell growth. Pemetrexed, like methotrexate, hinders multiple enzymes needed for de novo production of the thymidine and purine nucleotides, and is usually combined with cisplatin (an agent which promotes DNA cross-linking).

According to some embodiments of the invention the method further comprising administering to the subject an agent which inhibits the mammalian target of Rapamycin (mTOR) pathway.

Known inhibitors of mTOR pathway which can be used according to the method of some embodiments of the invention include, but are not limited to, Rapamycin and rapalogs (rapamycin derivatives).

According to some embodiments of the invention the method further comprising administering to the subject a therapeutically effective amount of Rapamycin.

Rapalogs which can be used according to the method of some embodiments of the invention include, but are not limited to, temsirolimus (CCI-779), everolimus (RAD001), and ridaforolimus (AP-23573), deforolimus (AP23573), everolimus (RAD001), and temsirolimus (CCI-779).

Temsirolimus is a pro-drug of rapamycin, and is a noncytotoxic agent which delays tumor proliferation. It is approved by the U.S. Food and Drug Administration (FDA) and the European Medicines Agency (EMA) for the treatment of renal cell carcinoma (RCC). Temsirolimus has higher water solubility than rapamycin and is therefore administrated by intravenous injection.

Everolimus is the second Rapamycin analog, approved by the U.S. FDA approved for the treatment of advanced renal cell carcinoma after failure of treatment with sunitinib or sorafenib, subependymal giant cell astrocytoma (SEGA) associated with Tuberous Sclerosis (TS), and Progressive neuroendocrine tumors of pancreatic origin (PNET); as well as for advanced hormone receptor-positive, HER2-negative breast cancer in combination with exemestane, and pediatric and adult patients with SEGA. It was also approved throughout the European Union for advanced breast cancer, pancreatic neuroendocrine tumours, advanced renal cell carcinoma, and SEGA in patients with tuberous sclerosis.

Deforolimus (AP23573, MK-8669), or ridaforolimus, is the newest rapamycin analog and it is not a prodrug. Like temsirolimus it can be administrated intravenously, and oral formulation is being estimated for treatment of sarcoma.

The second generation of mTOR inhibitors is known as ATP-competitive mTOR kinase inhibitors. The mTORC1/mTORC2 dual inhibitors are designed to compete with ATP in the catalytic site of mTOR. They inhibit all of the kinase-dependent functions of mTORC1 and mTORC2 and therefore, block the feedback activation of PI3K/AKT signaling, unlike rapalogs that only target mTORC1.

According to some embodiments of the invention the method further comprising administering to the subject a thymidine synthase inhibitor.

According to some embodiments of the invention the thymidine synthase inhibitor is Fluorouracil (5-FU), and/or Capecitabine.

Capecitabine is an oral 5-FU pro-drug. It is converted to 5-FU by actions in liver and tumor cells.

According to some embodiments of the invention the method further comprising over-expressing within tumor cells of the subject the Argininosuccinate synthase polypeptide (ASS1).

The ASS1 gene (Gene ID 445) is transcribed into two mRNA variants [variant 1, GenBank Accession No. NM_000050.4 (SEQ ID NO:43); and variant 2, GenBank Accession No. NM_054012.3 (SEQ ID NO:44)] which encode the same protein [GenBank Accession No. NP_000041.2 (SEQ ID NO:42)]. The ASS1 protein catalyzes the penultimate step of the arginine biosynthetic pathway.

Over-expression of ASS1 in tumor cells can be at the genomic level [i.e., activation of transcription via promoters, enhancers, regulatory elements, genome editing e.g., using homology directed repair (HDR), and/or by small molecules which can activate the ASS1 expression], at the transcript level (i.e., correct splicing, polyadenylation, activation of translation) or at the protein level (i.e., post-translational modifications, interaction with substrates and the like, and/or delivery of the ASS1 protein itself or of a functional portion thereof into the cells).

For example, upregulation of ASS1 can be performed by fisetin [Subramanian P., et al., 2014 (Pharmacological Reports 66 (2014) 1037-1042; which is fully incorporated herein by reference).

Upregulation of ASS1 can be also achieved using vectors (nucleic acid constructs) comprising an exogenous polynucleotide encoding ASS1 or a functional portion thereof, which are designed and constructed to express the ASS1 in the mammalian cells (preferably in tumor cells). Accordingly, the exogenous polynucleotide sequence may be a DNA or RNA sequence encoding an ASS1 molecule, capable of treating cancer.

The phrase “functional portion” as used herein refers to part of the ASS1 protein (i.e., a polypeptide) which exhibits functional properties of the enzyme such as argininosuccinate synthase catalytic activity. According to some embodiments of the invention the functional portion of ASS1 is a polypeptide sequence including amino acids 7-398 of the polypeptide set forth in SEQ ID NO:42, which is the region having the argininosuccinate synthase catalytic activity.

To express exogenous ASS1 in mammalian cells, a polynucleotide sequence encoding ASS1 [variant 1, GenBank Accession No. NM_000050.4 (SEQ ID NO:43); and variant 2, GenBank Accession No. NM_054012.3 (SEQ ID NO:44)] or a functional portion thereof is preferably ligated into a nucleic acid construct suitable for mammalian cell expression. Such a nucleic acid construct includes a promoter sequence for directing transcription of the polynucleotide sequence in the cell in a constitutive or inducible manner.

It will be appreciated that the nucleic acid construct of some embodiments of the invention can also utilize ASS1 homologues which exhibit the desired activity (i.e., argininosuccinate synthase catalytic activity). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO:42, as determined using the BestFit software of the Wisconsin sequence analysis package, utilizing the Smith and Waterman algorithm, where gap weight equals 50, length weight equals 3, average match equals 10 and average mismatch equals −9.

Constitutive promoters suitable for use with some embodiments of the invention are promoter sequences which are active under most environmental conditions and most types of cells such as the cytomegalovirus (CMV) and Rous sarcoma virus (RSV). Inducible promoters suitable for use with some embodiments of the invention include for example the tetracycline-inducible promoter (Zabala M, et al., Cancer Res. 2004, 64(8): 2799-804).

The nucleic acid construct of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed.

Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of ASS1 mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding a ASS1 can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMT010/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of ASS1 since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of some embodiments of the invention can also include sequences engineered to enhance stability, production, purification, or yield of the expressed peptide. For example, the expression of a fusion protein or a cleavable fusion protein comprising the ASS1 protein of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the ASS1 protein and the heterologous protein, the ASS1 protein can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

As mentioned, a purified ASS1 protein can be delivered to a cell, such as a tumor cell by ligating (by means of recombinant DNA) or conjugating the ASS1 to a cell-penetrating peptide which facilitates the entrance of the ASS1 to the cell, where the ASS1 functions. Cell-penetrating peptides are known in the art, and are further described hereinabove.

Each of the downregulating agents and/or the upregulating agents described hereinabove can be administered to the individual per se or as part of a pharmaceutical composition which also includes a physiologically acceptable carrier or excipients. The purpose of a pharmaceutical composition is to facilitate administration of the active ingredient to an organism.

According to an aspect of some embodiments of the invention there is provided a pharmaceutical composition comprising as an active ingredient a therapeutically effective amount of at least two distinct agents and a pharmaceutically acceptable carrier, wherein the at least two distinct agents selected:

down-regulating in tumor cells a mitochondrial aspartate-dependent pyrimidine synthesis; or

at least one of the at least two distinct agents down-regulating in tumor cells a mitochondrial aspartate-dependent pyrimidine synthesis and a second agent of the at least two distinct agents being an agent selected from the group consisting of: an agent for arginine depletion therapy, an agent for glutamine depletion, chemotherapy which inhibits production of nucleotide(s), an agent which inhibits the mammalian target of Rapamycin (mTOR) pathway, a thymidine synthase inhibitor, and an agent which over-expresses the Argininosuccinate synthase polypeptide.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent which downregulates a mitochondrial aspartate-dependent pyrimidine synthesis accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (an agent which downregulates a mitochondrial aspartate-dependent pyrimidine synthesis) effective to prevent, alleviate or ameliorate symptoms of the cancer or prolong the survival of the subject being treated.

Determination of a therapeutically effective amount is well within the capability of those skilled in the art, especially in light of the detailed disclosure provided herein. For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient in the tumor which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of cancer, as is further detailed above.

According to some embodiments of the invention the kit for treating cancer comprising at least two containers, the at least two containers separately packaging at least two distinct agents selected:

down-regulating in tumor cells a mitochondrial aspartate-dependent pyrimidine synthesis; or

at least one of the at least two distinct agents down-regulating in tumor cells a mitochondrial aspartate-dependent pyrimidine synthesis and a second agent of the at least two distinct agents being an agent selected from the group consisting of: an agent for arginine depletion therapy, an agent for glutamine depletion, chemotherapy which inhibits production of nucleotide(s), an agent which inhibits the mammalian target of Rapamycin (mTOR) pathway, a thymidine synthase inhibitor, and an agent which over-expresses the Argininosuccinate synthase polypeptide.

It should be noted that each of the containers of the kit can contain a distinct agent (thus the distinct agents contained in at least two containers are not the same). The agents can be both capable of down-regulating in tumor cells a mitochondrial aspartate-dependent pyrimidine synthesis. Additionally or alternatively, in one container the agent can be such that it is capable of down-regulating in tumor cells a mitochondrial aspartate-dependent pyrimidine synthesis, and in another container the agent can be such that it is selected from the group consisting of: an agent for arginine depletion therapy, an agent for glutamine depletion, chemotherapy which inhibits production of nucleotide(s), an agent which inhibits the mammalian target of Rapamycin (mTOR) pathway, a thymidine synthase inhibitor, and an agent which over-expresses the Argininosuccinate synthase polypeptide.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 2 is expressed in a DNA sequence format (e.g., reciting T for thymine), but it can refer to either a DNA sequence that corresponds to a citrin (aralar2) nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL METHODS

Orotic Acid Measurements in Human Subjects:

Urine samples were prepared by mixing 200 μl of a urine sample with isotopic internal standard ¹⁵N₂-orotic acid (Cambridge Isotope Laboratories). Orotic acid and orotidine were assayed on a Micromass Quattro mass spectrometer (Waters). HPLC was performed on a Waters ODS-AQ analytical column [150×2.0 mm (i.d.), 5-μm bead size]. Mobile phase was isocratic 0.05 M ammonium formate (pH 4.0). The MS/MS system was set at a flow rate of 0.2 ml/minutes. Mass spectrometer was operated in the Electrospray ionization (ESI) negative multiple-reaction-monitoring (MRM) mode. Nitrogen was used as nebulizer gas at flow rate of 60-90 l/hr (“1/hr” refers to liter(s) per hour) and desolvation gas 500 l/hr. Other optimized mass spectrometer parameters were cone voltage −15V, capillary −3250V and collision voltage −10V.

Genome Scale Metabolic Modeling (GSSM):

A metabolic network consisting of m metabolites and n reactions can be represented by a stoichiometric matrix S, where the entry sᵢⱼ represents the stoichiometric coefficient of metabolite i in reaction j 22. A CBM (Constraint-based modelling) model imposes mass balance, directionality and flux capacity constraints on the space of possible fluxes in the metabolic network's reactions through a set of linear equations:

S−v−0 (1)

vₘᵢₙ≤v≤vₘₐₓ (2)

Where v stands for the flux vector for all of the reactions in the model (i.e. the flux distribution). The exchange of metabolites with the environment is represented as a set of exchange (transport) reactions, enabling a pre-defined set of metabolites to be either taken up or secreted from the growth media. The steady-state assumption represented in Equation (1) constrains the production rate of each metabolite to be equal to its consumption rate. Enzymatic directionality and flux capacity constraints define lower and upper bounds on the fluxes and are embedded in Equation (2). In the following, flux vectors satisfying these conditions will be referred to as feasible steady-state flux distributions. The analyses were performed under the RPMI-1640 medium. The present inventors used the biomass function introduced in Folger et al. 2011.

Predicting Growth Rate, Metabolite Production and Flux Distribution Through Metabolic Modelling:

To determine the relation between ASS1 activity, CAD activity and growth rate, the present inventors utilized the generic human model and simulated the inactivation and activation of the reaction catalysed by ASS1. The inactivation was simulated by constraining the flux through the ASS1 reaction to zero, while the activation was simulated by enforcing increased positive flux through the ASS1 reaction up to the maximal possible flux, as computed via Flux Variability Analysis (Varma et al. 1994). At each such point, the maximal growth rate is computed via Flux Balance Analysis (FBA) (Varma et al. 1994). Additionally, the flux through the reaction catalysed by CAD under maximal growth rate was estimated based on 1000 different feasible flux samples (Bordel, S. et al., 2010).

Next, genome-scale metabolic models were utilized for each of the NCI-60 cancer cell lines based on their gene expression measurements (Yizhak, K. et al. 2014). In each cell-line model the following analyses were performed:

(1) the production of each biomass component under both the inactivation and maximal activation of ASS1 was computed as described above. The difference between the predicted production rates of each biomass component in the two states was then computed based on the results of this optimization problem.

(2) Similarly, the flux change of each reaction under maximal biomass production was examined in both the inactivation and activation states, as described above. In each of these states the solution space was sampled and 1000 feasible flux distributions were obtained (Bordel, S., et al., 2010). Focusing on the reactions associated with aspartate and glutamine, the fold-change in flux rate was computed together with its significance level. The latter was computed via a two-sided Wilcoxon rank sum test. The largest fold-change among these reactions was predicted for the reactions catalysed by the CAD enzyme.

TCGA Data Analysis:

For each tumor, normalized gene expression levels measured using RSEM (Lee, J. K. et al. 2007) were obtained from the RNASeqV2 data sets at the TCGA portal www://tcga-data(dot)nci(dot)nih(dot)gov/tcga/). Only matched tumor-normal pairs were used. For each tumor type, the mean expression levels in the tumor and normal samples were computed, a pseudo-count of 1 to each mean was added, and the ratio between the means was plotted.

Metabolomics Analysis:

Osteosarcoma or Melanoma cell lines were seeded at 10⁶ cells per 10 cm plates and incubated with either 4 mM L-GLUTAMINE (ALPHA-15N, 98%, Cambridge Isotope Laboratories, Inc.) or 4 mM L-GLUTAMINE (AMIDE-15N, 98%+, Cambridge Isotope Laboratories, Inc.) for 24 hours. Subsequently, cells were washed with ice cold saline, lysed with 50% methanol in water and quickly scraped followed by three freeze thaw cycles in liquid nitrogen. The insoluble material was pelleted in a cooled centrifuge (4° C.) and the supernatant was collected for consequent GC-MS analysis. The results were normalized to protein levels of each sample. Samples were dried under air flow at 42° C. using Techne Dry-Block Heater with sample concentrator (Bibby Scientific Limited, UK) and the dried samples were treated with 40 μl of a methoxyamine hydrochloride solution (20 mg/ml in pyridine) at 37° C. for 90 minutes while shaking followed by incubation with 70 μl N, O-Bis (trimethylsilyl) trifluoroacetamide (Sigma) at 37° C. for additional 30 minutes.

Gas Chromatography/Mass Spectrometry:

GC-MS analysis was performed using a gas chromatograph (7820AN, Agilent Technologies, USA) interfaced with a mass spectrometer (5975 Agilent Technologies, USA). A HP-5 ms capillary column 30 m×250 μm×0.25 μm (19091S-433, Agilent Technologies, USA) was used. Helium carrier gas was maintained at a constant flow rate of 1.0 mL min-1. The GC column temperature was programmed from 70 to 150° C. via a ramp of 4° C. min-1, 250-215° C. via a ramp of 9° C. min-1, 215-300° C. via a ramp of 25° C. min-1 and maintained at 300° C. for additional 5 minutes. The MS was by electron impact ionization and operated in full scan mode from m/z, 30-500. The inlet and MS transfer line temperatures were maintained at 280° C., and the ion source temperature was 250° C. Sample injection (1 μL) was in splitless mode.

Nucleic Acid Analysis

Materials:

Ammonium acetate (Fisher Scientific) and ammonium bicarbonate (Fluka) of LC-MS grade were used. Sodium salts of AMP, CMP, GMP, TMP and UMP were obtained from Sigma-Aldrich. Acetonitrile of LC grade was supplied from Merck. Water with resistivity 18.2 MΩ was obtained using Direct 3-Q UV system (Millipore).

Extract Preparation:

The obtained samples were concentrated in speedvac to eliminate methanol, and then lyophilized till dryness, re-suspended in 200 μl of water and purified on polymeric weak anion columns Strata-XL-AW 100u (30 mg/lml, Phenomenex) as following. Each column was conditioned by passing 1 ml of methanol, then 1 ml of formic acid/methanol/water (2/25/73), and equilibrated with 1 ml of water.

The samples were loaded, and each column was washed with 1 ml of water and ml of 50% methanol. The purified samples eluted with 1 ml of ammonia/methanol/water (2/25/73 ratios) followed by 1 ml of ammonia/methanol/water (2/50/50 ratios) were collected, concentrated in speedvac to remove methanol, and lyophilized. Before LCMC analysis the obtained residues were re-dissolved in 100 μl of water, centrifuged 5 minutes at 21,000 g to rid of insoluble material.

LCMS Analysis:

The LC-MS/MS instrument consisted of Acuity I-class UPLC system (Waters) and Xevo TQ-S triple quadruple mass spectrometer (Waters) equipped with an electrospray ion source and operated in positive ion mode was used for analysis of nucleoside monophosphates. MassLynx and TargetLynx software (v.4.1, Waters) were applied for the acquisition and analysis of data. Chromatographic separation was done on a 100×2.1-mm i.d., 1.8-μm UPLC HSS T3 column equipped with 50×2.1-mm i.d., 1.8-μm UPLC HSS T3 pre-column (both Waters Acuity) with mobile phases A (10 mM ammonium acetate and 5 mM ammonium hydrocarbonate buffer, pH 7.0 adjusted with 10% acetic acid) and B (acetonitrile) at a flow rate of 0.3 ml/min and column temperature 35° C. A gradient was as follows: 0-6 minutes the column was hold at 0% B, then 6-6.5 minutes linear increase till 100% B, 6.5-7.0 minutes hold at 100% B, 7.0-7.5 minutes back to 0% B and equilibration at 0% B for 2.5 minutes. Samples kept at 8° C. were automatically injected in a volume of 3 μl.

For mass spectrometry argon was used as the collision gas with flow 0.25 ml/minutes. The capillary voltage was set to 2.90 kV, source temperature −150° C., desolvation temperature −350° C., desolvation gas flow −650 L/minutes. Analytics were detected using multiple reaction monitoring (MRM) applying the parameters listed in Table 1 below.

TABLE 1
Parameters for multiple reaction monitoring (MRM)

| Name of | Retention time | | | |
|---|---|---|---|---|
| compound | (minutes) | Transition | Cone, V | CE, eV |
| AMP | 1.46 | 348.0 > 96.8 | 17 | 28 |
| | | 348.0 > 135.7 | 17 | 20 |
| CMP | 0.82 | 324.1 > 111.8 | 16 | 17 |
| GMP | 1.01 | 363.9 > 96.8 | 16 | 28 |
| | | 363.9 > 151.8 | 16 | 17 |
| TMP | 1.48 | 322.9 > 80.9 | 20 | 16 |
| UMP | 0.85 | 325.1 > 96.8 | 16 | 17 |

Table 1.

Hybridizations and Imaging:

Single molecule FISH (smFISH) was performed with probe libraries for Ass1 (74 probes, sequences described below) and Ki67 (96 probes) (Itzkovitz, S. et al. 2012). Imaging was performed as previously described (Itzkovitz, S. et al. 2012). smFISH images were filtered with a Laplacian of Gaussian filter of size 15 pixels and standard deviation of 1.5 pixels. Image is a maximum projection of 10 stacks spaced 0.3 um apart in the Z-direction.

Proximity Ligation Assay:

The assay was performed as published (Gu, G. J. et al. 2013) using Sigma Aldrich kit (Cat # DUO 92004-30-RXN). Antibodies used for detection were diluted in PBS; ASS1 (1:200, #ab170952, abcam), citrin (1:100, #H00010165-M01, clone #4F4, abnova) and anti-CAD (1:100, ab40800, abcam).

Cell Cultures:

Melanoma cell line LOX IMVI, MALME-3m and Osteosarcoma cell lines, MNNG/HOS, U2OS were purchased from ATTC and cultured using standard procedures in a 37° C. humidified incubator with 5% CO₂ in Roswell Park Memorial Institute Medium (RPMI) (Invitrogen) supplemented with 10% heat-inactivated fetal bovine serum and 2 mM glutamine.

Proliferation Assays

MTT:

Cells were seeded in 12-well plates at 8*10⁵ cells/well in a triplicate. After 6 hours for adherence of the cells, 0.1 mg/ml of MTT (3-(4,5-Dimethylthiazol-2-yl)-2,5 diphenyltetrazolium bromide) (Catalog No. CAS 298-93-1, Calbiochem) in dimethyl sulfoxide was added to each cell type, starting at 0 hour, in 24 hours intervals. Deoxynucleotide Set (Catalog No. DNTP100-1KT, Sigma-Aldrich) was added to the cells in the same time intervals at a final concentration of 10 μM. Absorbance was measured at 570 nm.

Crystal Violet Staining:

Cells were seeded in 12-well plates at 70,000 cells/well in a triplicate. Time 0 was calculated as the time the cells became adherent, which was after about 6 hours from plating. For each time point, cells were washed with PBS X1 and fixed in 4% PFA (in PBS). Cells were then stained with 0.1% Crystal Violet (Catalog #: C0775, Sigma-Aldrich) for 20 minutes (1 ml per well) and washed with water. Cells were then incubated with 10% acetic acid for 20 minutes with shaking. Extract was then diluted 1:4 in water and absorbance was measured at 590 nm every 24 hours.

RTCA:

Cells were seeded at a concentration of 2500 cells/well in microelectronic plates (E-Plates), coated with gold microelectrode arrays. Changes in impedance are measured at real-time, reflecting proportional changes in the number of cells inside the well. To determine which type of nucleotides contribute more to the proliferative state of cells over expressing ASS1, a mix of 10 μM dNTPs, 10 μM purines or 10 μM pyrimidine were added to the cells after adherence and in 24 hours intervals.

Protein and RNA Analysis

Western Blotting:

Cells were lysed in RIPA (Sigma-Aldrich) and 0.5% protease inhibitor cocktail (Calbiochem). Following centrifugation, the supernatant was collected and protein content was evaluated by the Bradford assay. 100 μg from each sample under reducing conditions were loaded into each lane and separated by electrophoresis on a 10% SDS polyacrylamide gel. Following electrophoresis, proteins were transferred to Immobilon transfer membranes (Tamar, Jerusalem, Israel). Nonspecific binding was blocked by incubation with TBST (10 mM Tris-HCl (pH 8.0), 150 mM NaCl, 0.1% Tween 20) containing 3% Albumin from Bovine Serum for 1 hour at room temperature.

Membranes were subsequently incubated with antibodies against ASS1 (1:500, sc-99178, Santa Cruz Biotechnology), p97 (1:10,000, PAS-22257, Thermo Scientific), GAPDH (1:1000, 14C10, #2118, Cell Signaling), CAD (1:1000, ab40800, abcam), phospho-CAD (Ser1859) (1:1000, #12662, Cell Signaling), p70 S6 Kinase (1:1000, #9202, Cell Signaling), phospho-p70 S6 Kinase (Ser371) (1:1000, #9208, Cell Signaling). Antibody was detected using peroxidase-conjugated AffiniPure goat anti-rabbit IgG or goat anti-mouse IgG (Jackson ImmunoResearch, West Grove, Pa.) and enhanced chemiluminescence western blotting detection reagents (EZ-Gel, Biological Industries).

Gels were quantified by Gel Doc™ XR+ (BioRad) and analyzed by ImageLab 4.1 software (BioRad). The band area was calculated by the intensity of the band. The obtained value was then divided by the value obtained from the loading control.

RNA Extraction and cDNA Synthesis:

RNA was extracted from cells by using

PerfectPure RNA Cultured Cell Kit (5′-PRIME). Complementary DNA was synthesized from lug RNA by using qScript™ cDNA Synthesis Kit (Quanta).

Quantitative Real-Time PCR:

Detection of ASS1 on cDNAs (see above) was performed using cyber green PCR master mix (Tamar, Jerusalem, Israel) and the required primers (Table 2 below). Analysis was performed using StepOne real-time PCR technology (Applied Biosystems, CA).

TABLE 2
Sequences of Primers used for quantitative
real-time PCR

| | | SEQ | | SEQ |
|---|---|---|---|---|
| | | ID | | ID |
| Gene | Forward Primer | NO: | Reverse Primer | NO: |
| Human | 5′-TTATAACCTGG | 5 | 5′-TGGACATAGCGT | 6 |
| ASS1 | GATGGGCACC-3′ | | CTGGGATTG-3′ | |
| Human | 5′-TGATGCCCCAG | 7 | 5′-TTGGTGAATCTG | 8 |
| ASL | AAGAAAAAC-3′ | | CAGCGTAG-3′ | |
| Human | 5′-GGGAACACTAC | 9 | 5′-GGCCTTTTCTTT | 10 |
| ARG1 | ATTTTGAAAACT-3′ | | CTTCCTAGTAG-3′ | |
| Human | 5′-ACCCTCCTGAAC | 11 | 5′-GGTCTTTGTCTC | 12 |
| ARG2 | ATTTTATT-3′ | | TTGCCAAT-3′ | |
| Human | 5′-GGCAATGGCTAC | 13 | 5′-CCCAAGCATGGT | 14 |
| CPS1 | AGGAAGAA-3′ | | ACCCTTATC-3′ | |
| Human | 5′-CTGAAGGGATGG | 15 | 5′-GGGTCCAAGTTC | 16 |
| CPS2 | AAGGAGATTG-3′ | | TCCATGTT-3′ | |
| Human | 5′-ATTGACACTGGC | 17 | 5′-TCCAACACTTCG | 18 |
| HPRT | AAAACAATGC-3′ | | TGGGGTCC-3′ | |

Transient Transfection:

Cells were seeded in 12-well plates at 30,000 cells/well, or in 10 cm plates at 10⁶ cells/plate, in a triplicate. At the following day, cells were transfected with either 20 pmol or 500 pmol siRNA siGenome SMARTpool targeted to ASS1 mRNA (Catalog #: M-010257-03-0020, Thermo scientific), respectively. Transfection was done with Lipofectamine® 2000 Reagent (Catalog No. 11668-019, Invitrogen) in the presence of Opti-MEM® I Reduced Serum Medium (Catalog No. 31985-062, Invitrogen). Four hours after transfection, medium was replaced and experiments were performed starting 24 hours post transfection.

Infection

Over-Expression:

Cells were infected with pLenti3.3/TR and with pLenti6.3/TO/V5-DEST-based lent viral vector with or without the human ASS1 transcript. Transduced cells were selected with 1 mg/ml Geneticin and with 7.5 μg/ml Blasticidin for each plasmid, respectively. When induction of expression was needed, cells were added with 10 μg/ml Tetracycline/Doxycycline.

shRNA:

Cells were infected with pLKO-based lenti viral vector with or without the human ASS1 shRNA encoding one or two separate sequences combined (Catalog No. RHS4533-EG445, GE Healthcare, Dharmacon). Transduced cells were selected with 2 μg/ml Puromycin.

ASS1 silencing was performed using the combination of two shRNA sequences:

(SEQ ID NO: 19)
1. 5′-AATGAGCATGGTAAAGGATGG-3′;
(SEQ ID NO: 20)
2. 5′-AACCGGTTGTAGAATTCAGGC-3′;

Arginine Deprivation Combined with Drug Treatments:

U2OS human osteosarcoma cell-line was seeded in 6-well plates at 80,000 cells/well. The following day, cells were treated with either 100 nM Rapamycin (#R0395, Sigma-Aldrich) or with 10 μM 5FU (#F6627, Sigma-Aldrich) in regular medium, with 10% dialyzed FCS-arginine-free-RPMI (Catalogue No. 06-1104-34-1A, Biological Industries, Kibbutz beit-haemek, Israel), or with both arginine depleted medium and one of these drugs. Rapamycin and 5FU were renewed into the medium every day, whereas fresh arginine-free medium was supplemented twice a week.

Animal Studies:

According to the approved IACUC protocol 17270415-2, 10⁷ MALME-3m melanoma cells suspended in 500 mcl (μl) with 5% Matrigel (Catalogue No. 4132053 Corning) were injected subcutaneously to 8-12 weeks old SCID and NSG mice that were purchased from Harlan. Mice were monitored for survival and tumor burden twice a week. Tumors were measured using a caliber. After euthanization, tumors were removed and incubated in medium containing ¹⁵N labeled glutamine for 6 hours followed by GCMS analysis.

Building Cell Models:

The present inventors utilized genome-scale metabolic models of NCI-60 cancer cell lines. The reconstruction method (based on the yet unpublished methods termed PRIME in Yizhak et al., personal communication) requires several key inputs: (a) the generic human model (Duarte, N. C. et al. 2007); (b) gene expression data for each cell line from (Lee et al., 2007), and (c) growth rate measurements (available at the NCI website: www://dtp(dot)nci(dot)nih(dot)gov/docs/misc/common_files/cell_list(dot)html). The algorithm then reconstructs a specific metabolic model for each sample by modifying the upper bounds of reactions in accordance with the expression of the individual gene microarray values.

Specifically, the model reconstruction process is as follows:

(1) Decompose reversible reactions into unidirectional forward and backward reactions.
(2) Evaluate the correlation between the expression of each reaction in the network and the measured growth rate. The expression of a reaction is defined as the mean over the expression of the enzymes catalyzing it.
(3) Modify upper bounds on reactions demonstrating significant correlation to the growth rate (after correcting for multiple hypothesis using FDR) in a manner that is linearly related to expression value.

FISH:

Ass1 probe (SEQ ID NO: 21) was used for single molecule FISH as shown in FIG. 1H.

Statistics:

All statistical analyses were performed using Tukey HSD or independent-samples Student's T-test of multiple or two groups, respectively. Log-transformed data were used where differences in variance were significant and variances were correlated with means. The sample size was chosen in advance based on common practice of the described experiment and is mentioned for each experiment. Each experiment was conducted with biological and technical replicates and repeated at least three times unless specified otherwise. Based on pre-established criteria, individual outlier data points that were more than 2 standard deviations away from the mean were excluded from the data analysis. Statistical tests were done using Statsoft's STATISTICA, ver. 10. All error bars represent SER. P<0.05 was considered significant in all analyses (* denotes P<0.05, **<P, 0.005, ***P<0.0005).

Kaplan Meir (KM)—

For each cancer type, the KM-plot indicates the survival rates of the 4 different patients group as labeled. Analysis was performed for the cancer types for which there was sufficient survival data.

### Example 1

### ASS1 Inactivation has an Important Role in Proliferation and that this is Related to Diversion of Aspartate Towards Pyrimidine Synthesis

To delineate the metabolic benefit(s) conferred by loss of ASS1 to cancers, the present inventors focused the studies on the relevant physiological and pathological model systems.

Without being bound by any theory, the present inventors have hypothesized that decreased ASS1 activity would enhance aspartate availability for CAD for the synthesis of pyrimidine nucleotides (FIG. 1A). Since increase in the pyrimidine pool is known to promote proliferation (Fairbanks, L. D. et al., 2995), the present inventors have predicted that cells with ASS1 deficiency would demonstrate enhanced proliferation. Furthermore, and without being bound by any theory, if this hypothesis is correct, deficiency in the mitochondrial aspartate transporter, citrin, would be expected to decrease aspartate availability for both ASS1 and CAD and hence restrict proliferation (FIG. 1A).

### Experimental Results

ASS1 Deficiency in CTLN I Results in Increased Synthesis of Pyrimidines Due to Increased Utilization of Aspartate by CAD—

As multiple lines of evidence suggest that cancer cells seize existing physiological mechanisms for their benefit (Fajas, L et al., 2013), the present inventors wanted to first test this hypothesis by assessing the correlation between ASS1 levels and proliferation in non-cancerous states. A generic stoichiometric model of human metabolism (Duarte, N. C. et al. 2007; Kobayashi, K., et al., 2005) predicted that inactivation of ASS1 is significantly associated with an increase in growth rate, and is additionally linked to increased flux through the reaction catalyzed by CAD (FIG. 1B). If this prediction was to be correct, it would be expected that subjects with CTLN I who are ASS1 deficient, would have increased synthesis of pyrimidines due to increased utilization of aspartate by CAD, as compared to those with CTLN II in whom aspartate availability to CAD is decreased (FIG. 1A). Indeed, urinary levels of orotic acid, a product reflecting the synthetic activity of CAD, were significantly elevated in human subjects with CTLN I as compared to the normative values from control population and subjects with CTLN II (FIGS. 1A and 1C).

CTLN I Fibroblasts have Increased Synthesis of Pyrimidines and Proliferation as Compared to CTLN II Cells—

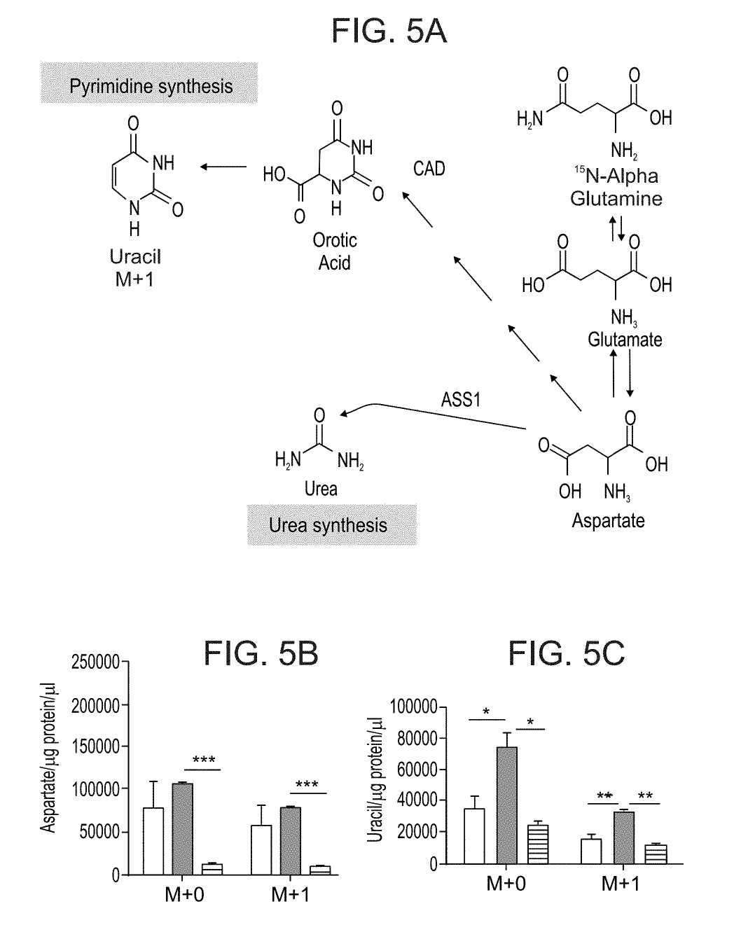

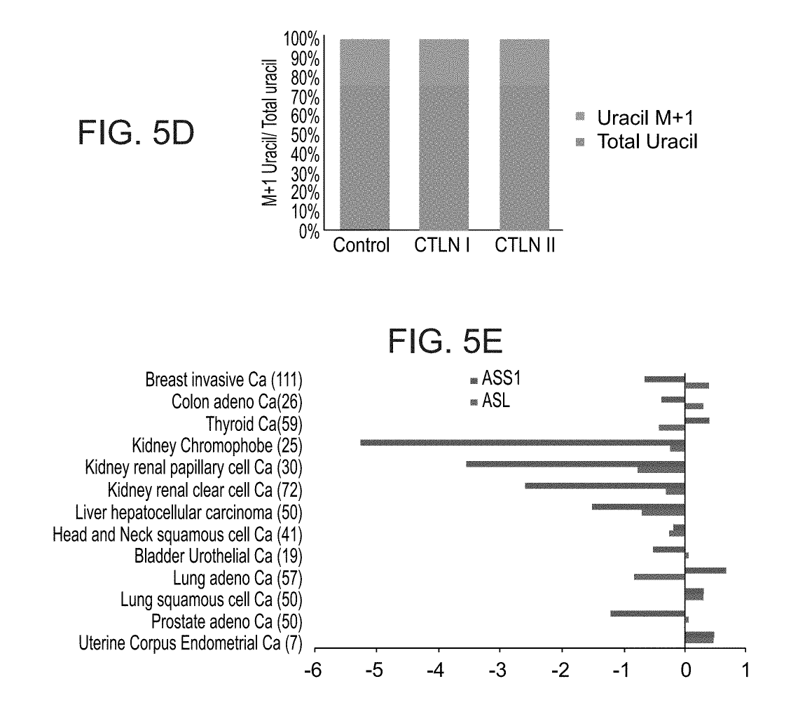

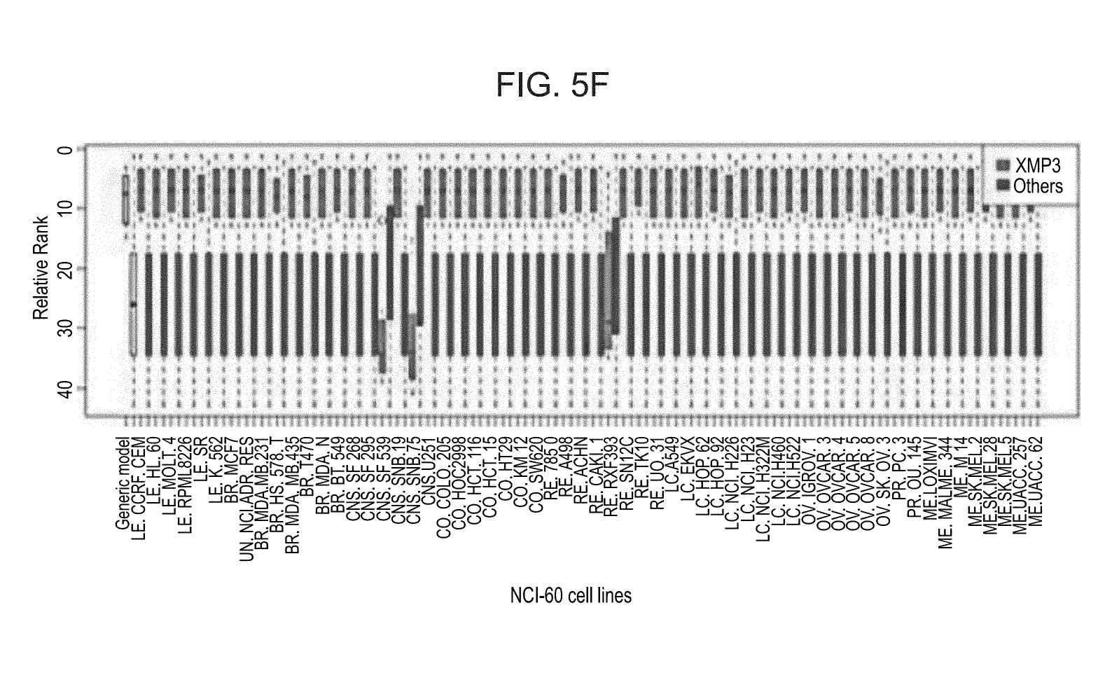

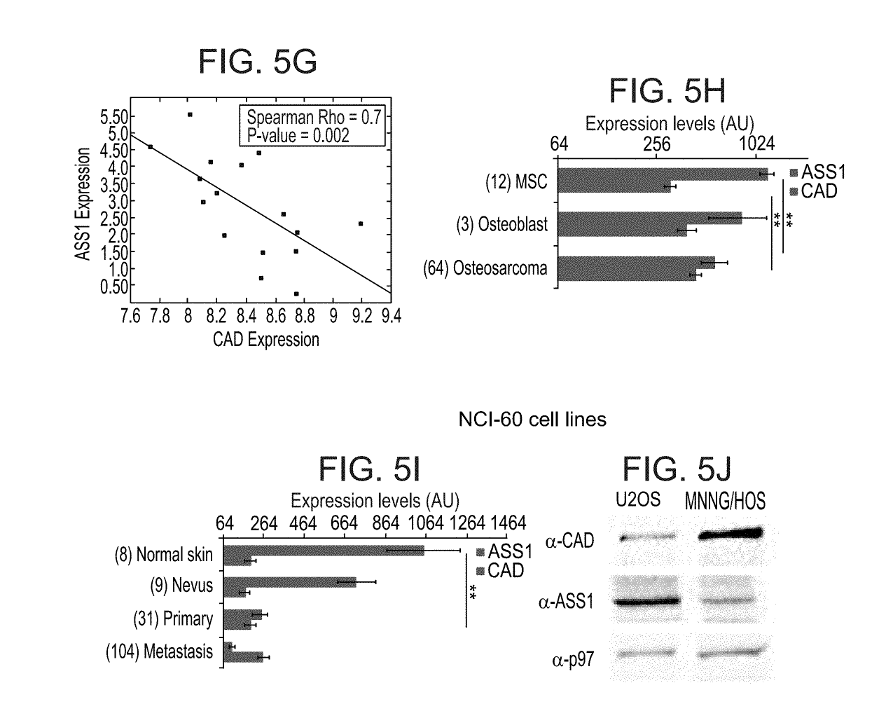

To further validate the consequences of loss of ASS1 activity, the present inventors studied primary fibroblasts from both subjects with CTLN I and CTLN II. The present inventors found that CTLN I fibroblasts have increased synthesis of pyrimidines and proliferation as compared to CTLN II cells (FIGS. 1D-E). To specifically trace the metabolic fate of mitochondrial-derived aspartate the present inventors used a stable isotopic form of glutamine, ¹⁵N₅-alpha glutamine that gets converted to ¹⁵N₅-aspartate in the mitochondria. This labeled nitrogen from aspartate is then incorporated into uracil which can be detected as M+luracil (FIG. 5A). Indeed, the present inventors show that CTLN I cells generate more total aspartate and total uracil as compared to control and CTLN II fibroblasts (FIGS. 1F-G). Hence, the specific decrease in transport of aspartate from the mitochondria caused by citrin deficiency in CTLN II, leads to reduced aspartate availability for pyrimidine synthesis and restricts proliferation in spite of sufficient aspartate levels in the medium. Interestingly, growth restriction has been reported in humans with CTLN II (Kobayashi, K., et al., 2005), but no growth aberrancies have been reported in CTLN I, further providing a clinical human context to the findings and suggesting that in physiological proliferation, aspartate deficiency has more severe clinical consequences than its enrichment.

Ass1 Expression is Distinctly Limited to the Differentiated Enterocytes of the Villi, while the Rapidly Proliferating Cells in the Intestinal Crypts do not Express Ass1—

To corroborate these results in another model system, the present inventors analyzed Ass1 mRNA levels in wild-type newborn mouse intestines. This tissue was chosen as it is known to express high levels of ASS1 and contains both proliferating and differentiating cells in the crypts and villi, respectively (Marion, V. et al. 2013). The present inventors found that Ass1 expression was distinctly limited to the differentiated enterocytes of the villi, while the rapidly proliferating cells in the intestinal crypts did not express Ass1 (FIGS. 1H-K). Moreover, there was a significant correlation between the levels of Ass1 and Glut2, a mature enterocyte marker in the differentiated enterocytes, whereas, a significant inverse correlation was observed between Ass1 and Ki-67 (a marker of proliferation) in the proliferating cells in the crypts (FIGS. 1J and 1K). Thus, the present inventors' modeling predictions, supported by human and murine studies, demonstrate that ASS1 inactivation has an important role in proliferation and that this is related to diversion of aspartate towards pyrimidine synthesis.

### Example 2

### Decreased Activity of ASS1 in Cancer Supports Proliferation by Activating Cad and Facilitating Pyrimidines Synthesis

### Experimental Results

Inverse Correlation Between ASS1 Expression Levels and Doubling Time of Cancer Cell Lines—

The present inventors next evaluated whether this mechanism to support cell proliferation could be the reason for the downregulation of ASS1 in cancer. As part of the “Warburg effect” described almost a 100 years ago (Warburg et al., 1956), cancer cells undergo metabolic transformations that allow for increased anabolic demands. According to this well-established phenomenon, different metabolites are diverted from their “routine pathways” for the synthesis of biological molecules that are essential for cell division and growth. To conduct an unbiased analysis of the possible role of ASS1 in cancer, the present inventors evaluated the ASS1 expression data in cancer cell lines from the NCI-60 collection. As shown in FIG. 2B, there was a significant inverse correlation between ASS1 expression levels and the reported doubling time of the cancerous cells. To further test whether the correlation between ASS1 expression and proliferation in cancer is explicable by diversion of aspartate flux, the present inventors utilized the current modeling program and predicted that with ASS1 inactivation, there is an accompanying significant increase in aspartate flux through the relevant metabolic reactions for nucleic acid synthesis (Table 3 hereinbelow). In contrast to inactivation of ASS1 in cancer, modeling the inactivation of ASL, the enzyme downstream of ASS1, predictably leads to endogenous arginine depletion that does not directly affect the flux towards nucleic acid synthesis (Data not shown). Furthermore, analysis of “The Cancer Genome Atlas (TCGA)” database for ASL and ASS1 expression shows that these genes can both be downregulated in the same cancers, suggesting that they are not mutually exclusive (FIG. 5B). Thus, both modeling and bioinformatics analyses support an arginine-independent effect for ASS1 silencing in cancerous proliferation that is related to nucleotide synthesis.

TABLE 3
ASS1 inactivation is predicted to increase aspartate
flux for nucleic acid synthesis

| Metabolic | Catalyzing | Inactive | |
|---|---|---|---|
| Pathway | Enzymes | ASS1/Active ASS1 | P-value |
| Pyrimidine | (790.1), CAD | ↑ | 8.47E−198 |
| Biosynthesis | | | |
| IMP Biosynthesis | (10606.1) PAICS | ↑ | <1E−300 |
| Nucleotides | (159.1) | ↑ | 1.55E−265 |
| | Adenylosuccinate | | |
| | synthease | | |

Table 3. Provided are the predicted fold-change in flux rates through pathways associated with aspartate and glutamine, when comparing ASS1 inactivation vs. activation state. The most significant change is predicted to effect the pyrimidine biosynthesis pathway followed by purine synthesis pathway, (two-sided Wilcoxon ranksum P-value <8.4e−198, Methods).

Cancerous Cell Lines Deficient in ASS1 had an Increase in Pyrimidine Levels and in Proliferation Rate as Compared to Cancerous Cells that have Higher Levels of ASS1—

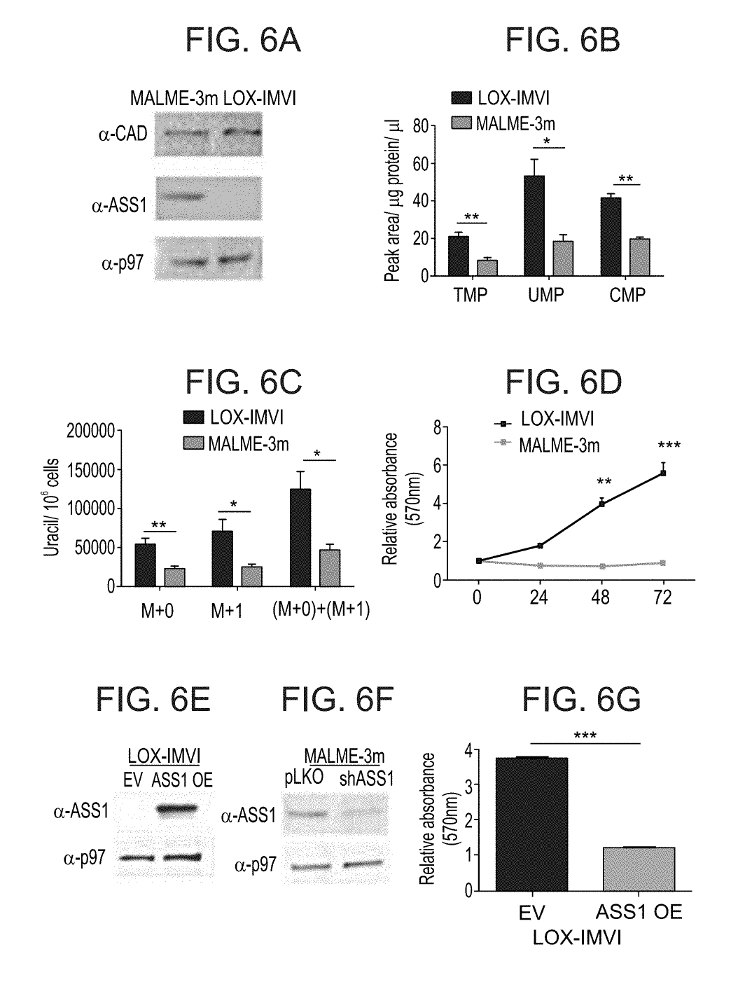

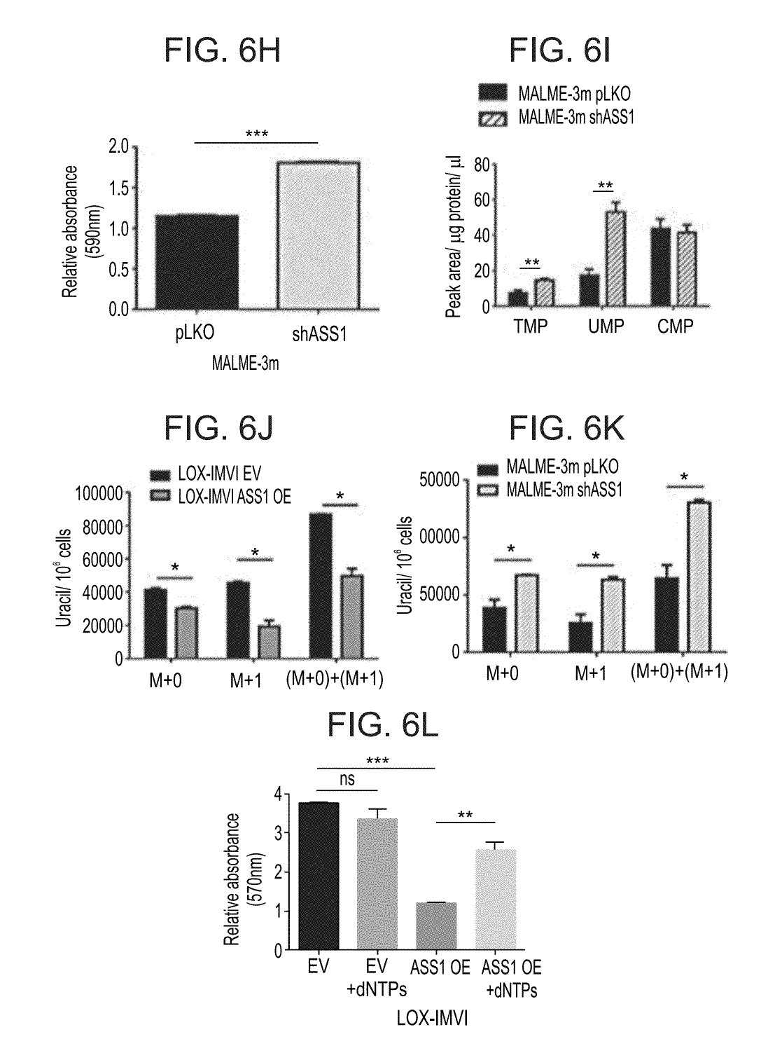

Using specific metabolic models tailored for each of the NCI-60 cell-lines (Yizhak, K. et al. 2014), the present inventors further predicted that 8 out of the 13 metabolites computationally shown to be increased with ASS1 inactivation, are nucleic acids (FIG. 2C and FIG. 5F). Additionally, specific analysis of the TCGA database of tumors where ASS1 expression is downregulated shows a significant upregulation in the expression of CAD, as compared to the paired normal tissue (FIG. 2D). The present inventors further confirmed the inverse upregulation in the expression of CAD versus ASS1 at the mRNA level in the NCI-60 cancer cell lines database as well as in specific databases for patients with osteosarcoma (Kuijjer, M. L. et al. 2013) and melanoma (Kabbarah, O. et al. 2010), and show that downregulation of ASS1 and upregulation of CAD are in concordance with cancerous phenotype (FIGS. 5G-H). In addition, the present inventors demonstrate the inverse expression levels between ASS1 and CAD at the protein level using osteosarcoma cell lines that differ in their expression pattern of ASS1 (FIG. 5J and FIG. 6A). To validate these modeling and global informatics analyses with experimental evidence, the present inventors studied osteosarcoma cell lines in which ASS1 was either deficient (MNNG/HOS) or present (U2OS) (FIG. 2E and FIG. 5A-J). Metabolic analysis confirmed that cells deficient in ASS1 had an increase in pyrimidine levels (FIG. 2F) as well as a significantly increased proliferation rate (FIG. 2H) as compared to osteosarcoma cells that have higher levels of ASS1. Furthermore, the level of total uracil that is generated from labeled glutamine is significantly higher in cancer cells with ASS1 deficiency (FIG. 2G). These results were additionally verified in melanoma cell lines that differ in their level of ASS1 expression (FIGS. 6B-D). Thus, analyses in both osteosarcoma and melanoma cells show that ASS1 downregulation is associated with an increase in aspartate availability for pyrimidine synthesis and with increased proliferation.

Changes in ASS1 Levels Alter the Proliferation Rate of Cancerous Cells—

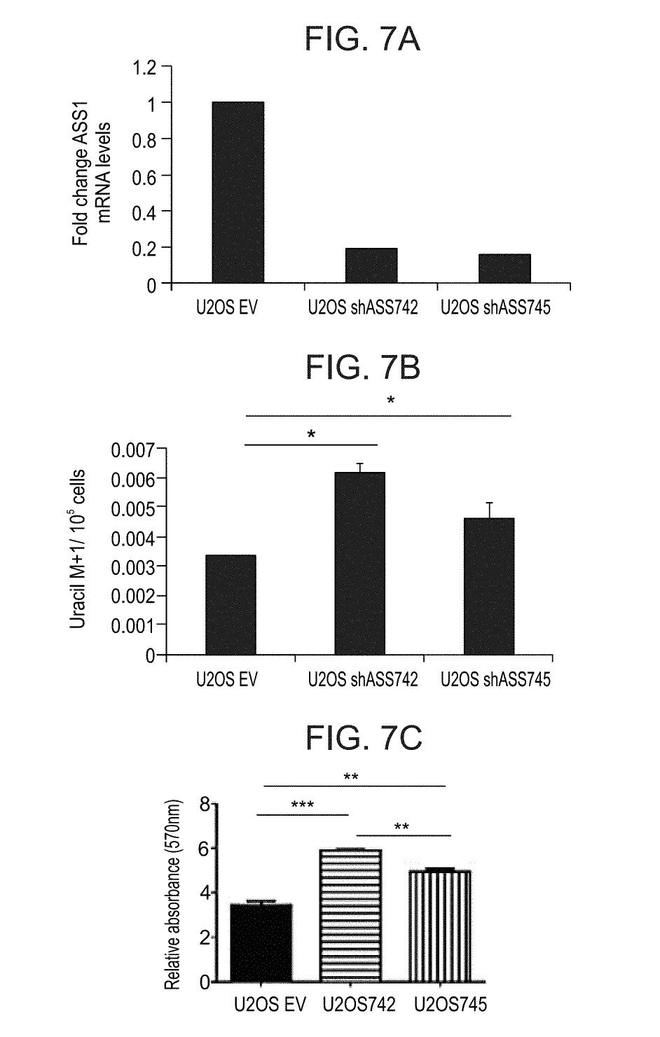

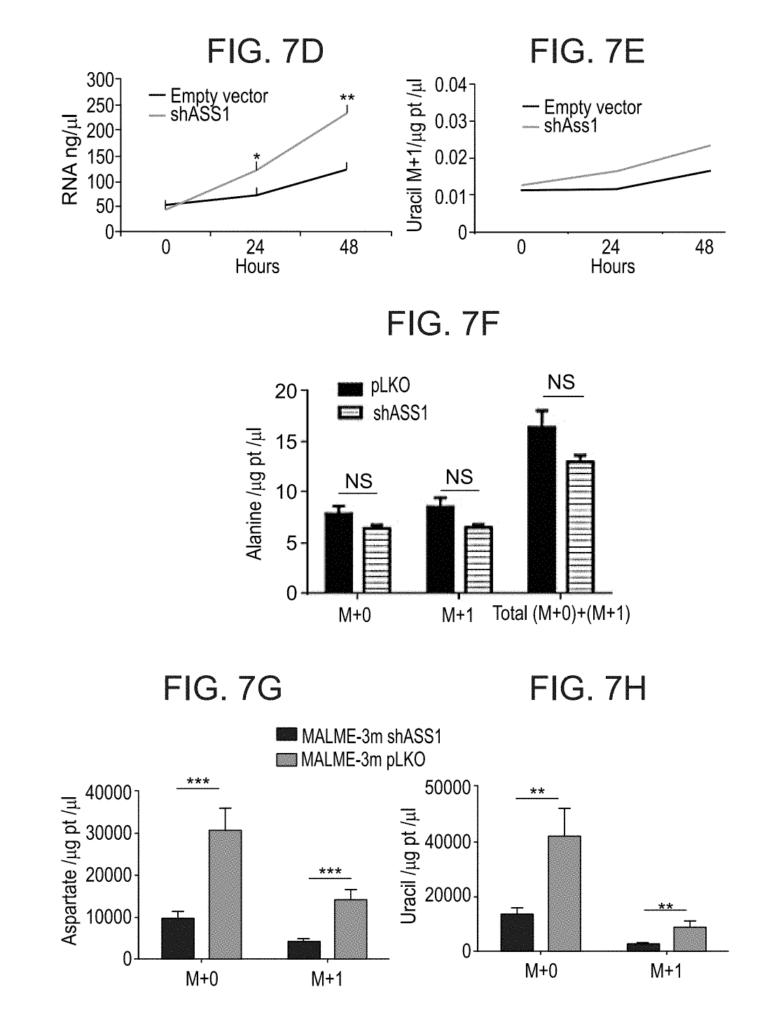

To more definitively dissect the connection between ASS1 expression and proliferation from all the other metabolic changes that occur in cancer cells, ASS1 was overexpressed in MNNG/HOS where it is endogenously downregulated, and was knocked down in U2OS cells where it is endogenously expressed (FIGS. 3A-B). The experiments clearly show that changes in ASS1 levels alter the proliferation rate of these cells; overexpressing ASS1 decreases proliferation while downregulation has the opposite effect (FIGS. 3C-D and FIGS. 7A-F). Furthermore, decreasing ASS1 levels resulted in increased pyrimidines levels (FIG. 3E) and overexpression of ASS1 decreased the total uracil levels (FIG. 3F).

Supplementation of Culture Media with Pyrimidines Restored the Proliferation of ASS1 Overexpressing Cells—

Without being bound by any theory the present inventors have hypothesized that if the major determinant by which ASS1 overexpression decreases proliferation is through diverting aspartate metabolism away from pyrimidine synthesis, supplementation with nucleic acids should restore proliferation. Indeed, supplementing the media with dNTP's and specifically with pyrimidines, significantly restored the proliferation of ASS1 overexpressing cells to a similar level as the parental cell-line (FIGS. 3G-I) as well as pyrimidines levels (FIG. 3I). Comparable results were obtained with manipulation of ASS1 levels in two melanoma cell lines as well (FIGS. 6E-K). Thus, in two distinct forms of cancers, changes in ASS1 expression levels directly affect aspartate utilization for pyrimidine synthesis and proliferation. Importantly, similar results were obtained in-vivo where mice injected with melanoma cells knocked down for ASS1, developed tumors that grew more rapidly and had higher levels of total and M+1 labeled aspartate and uracil as compared to the parental tumor cells that expressed the empty vector (FIG. 3J-M and FIG. 7G).

Downregulation of ASS1 in Melanoma Cells Resulted in Faster Growth of Tumors In Vivo—

To test whether these in vitro studies could be replicated in a relevant in vivo model, the present inventors injected the melanoma cells with and without ASS1 knockdown to immune deficient mice. Mice injected with melanoma cells knocked down for ASS1, developed tumors which were characterized by increased tumor volume and rapid growth and had higher levels of total uracil as compared to the parental tumor cells that expressed the empty vector (FIGS. 3J-M).

These results show that decreased activity of ASS1 in cancers supports proliferation by activating CAD (carbamoyl-phosphate synthase 2, aspartate transcarbamylase, dihydroorotase complex) and facilitating pyrimidines synthesis.

### Example 3

### Downregulation of Citrin in Cancerous Cells Decreases Pyrimidine Levels

### Experimental Results

Citrin is Upregulated in Cancerous Tissues—

The present inventors have envisaged that a synergistic way to increase aspartate delivery for pyrimidine synthesis would be by upregulation of citrin. Further analysis of the TCGA data showed that in tissues that normally do not express citrin at high levels (del Arco et al., 2002), there is significantly elevated expression in the cancerous state (FIGS. 8A-B and FIG. 12).

Downregulation of Citrin in Cancerous Cells Resulted in Decreased Pyrimidine Levels and Decreased Total Aspartate and Aspartate-Derived Total Orotic Acid Levels—

In addition, in the liver where citrin is strongly expressed, a recent publication of ASS1 expression in hepatocellular carcinoma showed that downregulation of ASS1 is associated with a more malignant cancerous phenotype (Tan et al., 2014). These results, together with the results of the present study of primary human fibroblast cells that demonstrate that loss of citrin restricts proliferation (FIGS. 1C-G), are important as they imply that proliferation induced by the loss of ASS1 in the tumor might be counteracted by inhibiting citrin. Indeed, using siRNA targeting citrin in U2OS, the present inventors were able to decrease proliferation independent of ASS1 levels, but the effect was more robust when ASS1 levels were decreased (FIG. 4A). Use of si-citrin decreased pyrimidine levels in the context of ASS1 downregulation and furthermore, cells transduced with shASS and transfected with si-citrin demonstrate a decrease in total aspartate as well as aspartate-derived total orotic acid levels (FIGS. 4B-D and FIGS. 8C-E). These results indicate that mitochondrial derived aspartate is not only an important substrate for pyrimidine synthesis but is crucial in cancers with ASS1 downregulation.

As citrin is part of the malate-aspartate shuttle, its deficiency is expected to affect several aspects in cell survival and growth. The results described herein indicate that citrin function in transferring mitochondrial-derived aspartate is important for supplying substrate for pyrimidine synthesis, especially in cancers with ASS1 downregulation. These findings are therapeutically relevant as survival analysis of several cancers in the TCGA database reveal that cancers with both decreased ASS1 expression and high citrin levels have a trend for significantly worse prognosis (FIGS. 4E-F, FIG. 8F-G and Table 4).

Downregulation of citrin decreases S6K1 and CAD phosphorylation—The utilization of citrin-derived aspartate by CAD following ASS1 downregulation requires activation of CAD. Recently, it was described that CAD is activated via phosphorylation by ribosomal protein S6 kinase (S6K1), a process that is regulated by the mTOR pathway (Robitaille, A. M. et al. 2013; Ben-Sahra, I., et al., 2013). The present inventors found that when ASS1 expression in cancer cells is decreased, there is increased phosphorylation of S6K1 and CAD (FIG. 4G). Furthermore, when availability of mitochondrial-exported aspartate is decreased by si-citrin, S6K1 and CAD phosphorylation is also decreased (FIG. 4G). Since blockade of citrin-derived aspartate prevents the activation of CAD through the mTOR pathway even in the presence of ASS1 downregulation, the results of the present study imply that aspartate levels are important in regulating the mTOR pathway signaling towards pyrimidine synthesis.

ASS1 Downregulation Results in Increase in Location Proximity Between CAD and Citrin—

Finally, to demonstrate the competition between ASS1 and CAD for aspartate as substrate, the present inventors performed proximity ligation assay that show a significant increase in the location proximity between CAD and citrin following ASS1 downregulation (FIGS. 8F-I). These results together suggest that aspartate levels determine CAD activation and pyrimidine levels by regulating CAD's substrate availability, protein localization, and activity. In complete concordance with data showing that upregulation of ASS1 is associated with decreased response to mTOR inhibitors (Long, Y. et al. 2013) there is a decrease in proliferation when ASS1 deficient cells are treated with mTOR inhibitor Rapamycin (FIG. 4H). Use of Rapamycin also results in decreased CAD activation in ASS1 deficient cells (FIG. 4I). Furthermore, adding the thymidine synthase—Fluorouracil (5FU) to cancer cells with ASS1 deficiency grown in arginine depleted medium, has a significant additive beneficial effect (FIG. 4H). Hence, mTOR and thymidine synthase inhibitors could both have a favorable effect when added to arginine depletion therapy which is the current therapeutic modality for ASS1 depleted cancers (FIG. 4J).

In summary, these studies demonstrate that ASS1, a urea cycle enzyme, regulates pyrimidine synthesis in cancerous proliferation by regulating CAD activation via regulating aspartate levels.

### Example 4

Materials and Experimental Methods

Cell Cultures—

All cell lines were authenticated. Melanoma cell lines LOX-IMVI and MDA-MB-435 and ovarian cancer cell line SKOV3 were purchased from American Type Culture Collection (ATCC) and cultured using standard procedures in a 37° C. humidified incubator with 5% CO₂ in RPMI (Invitrogen) or DMEM supplemented with 10% heat-inactivated fetal bovine serum, 10% pen-strep and 2 mM glutamine. All cells are tested routinely for mycoplasma using a Mycoplasma EZ-PCR test kit (20-700-20, Biological Industries).

Normal control or CTLN-II-derived fibroblasts were given by the collaboration with Prof. David Dimmock and his student Daniel Helbling from the Human and Molecular Genetic and Biochemistry Center, Medical College Wis., Milwaukee, Wis. Cells were cultured in DMEM supplemented with 20% heat-inactivated fetal bovine serum, 10% pen-strep and 2 mM glutamine.

Metabolomics Analysis—

Melanoma cell lines were seeded at 3×10⁶ to 5×10⁶ cells per 10 cm plate and incubated with 2 g/L D-glucose (U-13C6, 99%) (Cat. Number: CLM-1396-10) for 2 hours. Normal or CTLN-II-derived fibroblasts were incubated with 2 g/L D-glucose (U-13C6, 99%) at 80%-90% confluence for 24 hours. Subsequently, cells were washed with ice-cold saline, lysed with 50% methanol in water and quickly scraped followed by three freeze-thaw cycles in liquid nitrogen. The insoluble material was pelleted in a cooled centrifuge (4° C.) and the supernatant was collected for consequent GC-MS analysis. Samples were dried under air flow at 42° C. using a Techne Dry-Block Heater with sample concentrator (Bibby Scientific) and the dried samples were treated with 40 μl of a methoxyamine hydrochloride solution (20 mg ml⁻¹ in pyridine) at 37° C. for 90 minutes while shaking followed by incubation with 70 μl N,O-bis (trimethylsilyl) trifluoroacetamide (Sigma) at 37° C. for an additional 30 minutes.

GC-MS—

GC-MS analysis used a gas chromatograph (7820AN, Agilent Technologies) interfaced with a mass spectrometer (5975 Agilent Technologies). An HP-5 ms capillary column 30 m×250 μm×0.25 μm (19091S-433, Agilent Technologies) was used. Helium carrier gas was maintained at a constant flow rate of 1.0 ml min −1. The GC column temperature was programmed from 70 to 150° C. via a ramp of 4° C. min −1, 250-215° C. via a ramp of 9° C. min −1, 215-300° C. via a ramp of 25° C. min −1 and maintained at 300° C. for an additional 5 min. The MS was by electron impact ionization and operated in full-scan mode from m/z=30-500. The inlet and MS transfer line temperatures were maintained at 280° C., and the ion source temperature was 250° C. Sample injection (1 μl) was in splitless mode.

Crystal Violet Staining—

Cells were seeded in 12-well plates at 40,000-100,000 cells per well in a triplicate. Time 0 was calculated as the time the cells became adherent, which was after about 6 hours from plating. After 6 days, cells were washed with PBS X1 and fixed in 4% PFA (in PBS). Cells were then stained with 0.1% Crystal Violet (C0775, Sigma-Aldrich) for 20 minutes (1 ml per well) and washed with water. Cells were then incubated with 10% acetic acid for 20 minutes with shaking. Extract was then diluted 1:4 in water and absorbance was measured at 590 nm.

Protein and RNA Analysis:

Western Blotting—

Cells were lysed in RIPA (Sigma-Aldrich) and 0.5% protease inhibitor cocktail (Calbiochem). After centrifugation, the supernatant was collected and protein content was evaluated by the Bradford assay. One hundred micrograms from each sample under reducing conditions were loaded into each lane and separated by electrophoresis on a 10% SDS polyacrylamide gel. After electrophoresis, proteins were transferred to Immobilon transfer membranes (Tamar). Non-specific binding was blocked by incubation with TBST (10 mM Tris-HCl (pH 8.0), 150 mM NaCl, 0.1% Tween 20), containing 3% albumin from bovine serum for 1 hour at 25° C. Membranes were subsequently incubated with antibodies against Citrin (1:200, H00010165-M01, Abnova), ASS1 (1:500, sc-99178, Santa Cruz Biotechnology) and p97 (1:10,000, PA5-22257, Thermo Scientific). Antibody was detected using peroxidase-conjugated AffiniPure goat anti-rabbit IgG or goat anti-mouse IgG (Jackson ImmunoResearch) and enhanced chemiluminescence Western blotting detection reagents (EZ-Gel, Biological Industries). Gels were quantified by Gel Doc XR+(BioRad) and analysed by ImageLab 4.1 software (BioRad). The band area was calculated by the intensity of the band. The obtained value was then divided by the value obtained from the loading control.

RNA Extraction and Complementary DNA (cDNA) Synthesis—

RNA was extracted from cells by using PerfectPure RNA Cultured Cell Kit (5′-PRIME). cDNA was synthesized from 1 μg RNA by using qScript cDNA Synthesis Kit (Quanta).

Quantitative PCR—

Detection of Citrin on cDNAs (see above) was performed using SYBR green PCR master mix (Tamar) and the required primers. Primer sequences were as follows. Human Citrin: forward, 5′-TGAGCCAAGGGGACGTATGA-3′ (SEQ ID NO:22); reverse, 5′-CGAGCTGAATCACCTGAGGC-3′ (SEQ ID NO:23). Human HPRT: forward, 5′-ATTGACACTGGCAAAACAATGC-3′ (SEQ ID NO:24); reverse: 5′-TCCAACACTTCGTGGGGTCC-3′ (SEQ ID NO:25). Analysis used StepOne real-time PCR technology (Applied Biosystems).

Short Hairpin RNA—

Cells were infected with SMARTvector inducible lentiviral shRNA vector with or without the human Citrin short hairpin RNA (shRNA) encoding two separate sequences (Catalogue numbers VSH6376220882448 (sh901, clone ID Number V2IHSHER_148901) and VSH6376220882456 (sh909, clone ID Number V2IHSHER_148909) and VSH6376220882464 (sh917, clone ID Number V2IHSHER_148917) 100 μL, GE Healthcare, Dharmacon). Transduced cells were selected with 2 μg ml⁻¹ puromycin.

Citrin silencing was performed by two separate complementary sequences:

(SEQ ID NO: 26)
1. sh901-5′-TTAAGAAAGTGCTACGCTA-3′;
(SEQ ID NO: 27)
2. sh909-5′-GTATCCTATCGATCTTGTA-3′;
(SEQ ID NO: 45)
3. sh917-5′-TGGGAGAACTCATGTATAA-3′;

Over-Expression—

Cells were infected with either pLEX_307 empty vector or with human Citrin (GenBank Accession No. NM_001160210.1; SEQ ID NO:2) under EF1-alpha promoter. Transduced cells were selected with 2 μg ml⁻¹ puromycin.

Transmission EM (Electron Microscopy)—

Normal control, CTLN-II-derived fibroblasts and melanoma cell-line LOX-IMVI EV and Citrin over-expression were fixed with 3% paraformaldehyde, 2% glutaraldehyde in 0.1 M cacodylate buffer containing 5 mM CaCl₂(pH 7.4). Next, samples were post-fixated in 1% osmium tetroxide supplemented with 0.5% potassium hexacyanoferrate tryhidrate and potassium dichromate in 0.1 M cacodylate (1 hour), stained with 2% uranyl acetate in water (1 hour), dehydrated in graded ethanol solutions, and embedded in Agar 100 epoxy resin (Agar Scientific). Ultrathin longitudinal sections (70 nm) were viewed and photographed with a Tecnai Spirit (FEI) transmission electron microscope operated at 120 kV and equipped with an Eagle CCD (charge-coupled device) camera.

Database Content—

MERAV database was assembled from the human gene expression data obtained from the NCBI GEO repository. In particular, the present inventors manually curated Affymetrix U133 Plus 2.0 arrays (GPL570 platform in GEO). This platform was chosen over other Affymetrix designs because it includes a relatively recent set of probes and comprises a wide range of experiments (115,886 in GEO). The assembled arrays reflect the human gene expression in normal tissues, cancer cell lines and primary tumors, and were collected from the following sources: (i) Cancer Cell Line Encyclopedia (CCLE), a joint project between Novartis and the Broad Institute, representing the expression of 729 cell lines; (ii) GlaxoSmithKline (GSK) representing the expression of 870 cell lines; (iii) Expression Project for Oncology (ExpO), a gene expression database representing the expression of 1,312 primary tumors generated by the International Genomic Consortium (GEO accession: GSE2109); (iv) Human Body Index (HBI) that represents the expression of 426 normal human tissues (GEO accession: GSE7307); (v) Gene Expression Omnibus database (GEO), human microarray data is publicly available from the NCBI GEO database. In order to retrieve the GEO arrays the present inventors manually searched the NCBI GEO dataset for the most relevant experiments. This dataset includes gene expression data from normal tissues (N, 317 arrays), primary tumors (P, 292 arrays) and cancer cell lines (C, 508 arrays) and were labeled GEO-N, GEO-P, GEO-C respectively [Shaul, Y. D. et al. MERAV: a tool for comparing gene expression across human tissues and cell types. Nucleic Acids Res 44, D560-566, doi:10.1093/nar/gkv1337 (2016)].

ATP Synthesis Assay—

Modified from B. H Robinson et al. Am J Hum Genet, 1985. Normal control and CTLN-II-derived fibroblasts were grown in T-25 cm² and received with 1 ml of medium containing 0.25M sucrose, 20 mM MOPS pH 7.4 and 0.1 mg/ml digitonin (Buffer A). After 5 minutes incubation, media was removed and cells were added with 1 ml of a solution containing 0.25 M sucrose, 20 mM EDTA and 20 mM MOPS, pH 7.4 (Buffer B). After 5 minutes incubation, media was removed and cells were added with medium containing 0.25 M sucrose, 25 mM MOPS, pH7.4, 1 mM EDTA, 5 mM KPi and 1 mM ADP (Buffer C). This is the “No substrate Media”. Then cells were added with ATP's/reducing equivalent (5 mM pyruvate/O.3 mM L-malate). After one hour incubation with substrates, fibroblast monolayer is sonicated and scraped off the flask, and treated with 0.05 volume of 1.6 M perchloric acid. The resulting mixture is centrifuged at 20,000 rpm for 20 minutes to remove protein. The supernatant is assayed by fluorometric method for ATP. The protein pellet is re-suspended in any appropriate buffer and samples taken for protein determination [Robinson, B. H., McKay, N., Goodyer, P. & Lancaster, G. Defective intramitochondrial NADH oxidation in skin fibroblasts from an infant with fatal neonatal lacticacidemia. Am J Hum Genet 37, 938-946 (1985)].

### Experimental Results

Citrin Deficient Fibroblasts have Decreased Glycolysis and Decreased ATP and NADH Production—

Citrin-deficiency in fibroblasts results in significant decreases in lactate reflecting decreased glycolysis as compared to control fibroblasts (FIG. 9A), decreased mitochondrial NADH production by citrin deficient fibroblasts as compared to control fibroblasts (FIG. 9B) and produce less ATP reflecting less oxydative phosphorylation compared to control fibroblasts (FIG. 9C).

Citrin Deficient Fibroblasts May have Increased Autophagy—

Citrin deficient fibroblasts were obtained from CTLN II patients. Control and citrin-deficient fibroblasts were subjected to electron microscopy analysis and the results show that citrin-deficient fibroblasts have increased autophagy (FIG. 10B) as compared to control fibroblasts (FIG. 10A).

Silencing Citrin in Cancer Decreases Cells' Proliferation—

Cancer cells (MDA-MB-435 melanoma cancer cell-line) were subjected to shcitrin in order to downregulate the level of citrin in the cells. As shown in the representative Western blot analysis cells transfected with the sh901 and sh909 citrin silencing shRNA exhibit no production of citrin (FIG. 11A). Quantitative RT-PCR analysis showed that in cells transfected with the shcitrin under the induction of doxycycline (Dox) the level of citrin RNA was signficantly reduced. The citrin-inhibited cells (treated with shcitrin under induction of Dox) exhibit a significant decrease in cell proliferation as compared to control cells infected with an empty vector (EV) (FIG. 11C).

Citrin is Over Expressed Specifically in Hepatocytes and Cancer Cells—

Using the MERAV database and computational analysis of Citrin expression in normal and cancerous cells and tissues (TCGA database) the present inventors have found significant difference in the expression level of citrin in tissues with a high base line expression of citrin (FIG. 8A) as compared to the significant elevation in tumors in which the normal tissue has low basal expression of citrin (FIG. 8B).

Over-Expression of Citrin Results in Enhanced Energy Production—

The melanoma cell line LOX-IMVI and the ovarian cancer cell line SKOV3 were transfected to over-express the citrin coding sequence under the transcriptional regulation of EF1-alpha promoter. As shown in FIG. 13A, Western blot analysis confirmed the over-expression of the citrin protein in both cancerous cell lines as compared to control cancerous cell lines that were transfected with an empty vector. Next, the state of energy production in the cells over-expressing citrin was evaluated by analysis of lactate production (FIG. 13B) and mitochondrial size (FIGS. 14A-D). The results conclusively show that citrin over expressing cancer cells have increased lactate production (indicating increased glycolysis) (FIG. 13B) and enlarged mitochondrias (FIGS. 14C-D) as compared to cancerous cells which do not over-express citrin (i.e., the same cancerous cell lines which were transfected with an empty vector), suggesting enhanced energy production as a result of citrin over-expression.

Analysis and Discussion—

Citrin is over expressed in cancer as it is required for cancer cells' energy production by enhancing glycolysis and NADH synthesis, as well as for transporting aspartate. When Citrin is deficient, cancer cells show decreased proliferation and go through autophgocytosis. Thus, targeting citrin as cancer therapy could be beneficial for treating cancers.

### Example 5

### The Effect of Citrin Inhibitors on Cancer Cell Proliferation

Materials and Experimental Methods

Metabolomics Analysis for Potential Citrin Inhibitors—

Osteosarcoma cell line (U2OS) were seeded at 1.5 to 2×10⁶ cells per 10 cm plate. The next day (60% confluence) the cells were incubated with 4 mM of L-Glutamine (ALPHA-15N, 98%; Cat. Number: NLM-1016-1; Cambridge Isotope Laboratories) in the presence of either control (medium only) or one of compounds 1, 2, or 3 (at a concentration range between 10-100 μM), for 24 hours. Compound “1”=D-Glutamic acid; compound “2”=L-glutamic acid-5-methyl ester; “compound “3”=L-glutamic acid-gamma-benzyl ester. Compounds 1-3 are commercially available and were bought from Sigma Aldrich. Subsequently, cells were washed with ice-cold saline, lysed with 50% methanol in water and quickly scraped followed by three freeze-thaw cycles in liquid nitrogen. The insoluble material was pelleted in a cooled centrifuge (4° C.) and the supernatant was collected for consequent GC-MS analysis. Samples were dried under air flow at 42° C. using a Techne Dry-Block Heater with sample concentrator (Bibby Scientific) and the dried samples were treated with 40 μl of a methoxyamine hydrochloride solution (20 mg ml⁻¹ in pyridine) at 37° C. for 90 minutes while shaking followed by incubation with 70 μl N,O-bis (trimethylsilyl) trifluoroacetamide (Sigma) at 37° C. for an additional 30 minutes.

GC-MS—

GC-MS analysis used a gas chromatograph (7820AN, Agilent Technologies) interfaced with a mass spectrometer (5975 Agilent Technologies). An HP-5 ms capillary column 30 m×250 μm×0.25μm (19091S-433, Agilent Technologies) was used. Helium carrier gas was maintained at a constant flow rate of 1.0 ml min −1. The GC column temperature was programmed from 70 to 150° C. via a ramp of 4° C. min −1, 250-215° C. via a ramp of 9° C. min −1, 215-300° C. via a ramp of 25° C. min −1 and maintained at 300° C. for an additional 5 minutes. The MS was by electron impact ionization and operated in full-scan mode from m/z=30-500. The inlet and MS transfer line temperatures were maintained at 280° C., and the ion source temperature was 250° C. Sample injection (1 μl) was in splitless mode.

### Experimental Results

Prediction of the Potential of Candidate Compounds to Inhibit Citrin—

The present inventors have tested several candidate compounds by chemoinformatics using a docking approach (grid generation based on SiteMap and 1OKC ligand; Halgren T A., et al., 2009 (J. Chem. Inf. Model, 49: 377-389; Osguthorpe D J., et al., 2012. Chem. Biol. Drug 80: 182-193; Halgren T. 2007. Chem. Biol. Drug Des. 69: 146-148; each of which is fully incorporated herein by reference in its entirety) for their ability to inhibit the activity of citrin as a protein carrier. Compound “3” which is L-Glutamic acid γ-benzyl ester (CAS Number: 1676-73-9) got the highest docking rank score in terms of its potential to inhibit Citrin. Compound “3” occupies the SiteMap grid and make h-bond and pi stacking. H-Bond 5166, E100, N81 and R85 Pi stacking/T-shape with F165/Y169.

Testing the Ability of the Candidate Citrin Inhibitors to Inhibit Citrin in Cells—

The present inventors have tested three compounds which could potentially inhibit Citrin as described in the methods section above, Briefly, Osteosarcoma cells (U2OS) were labeled with 15-N(Alpha) Glutamine and the cells were monitored for presence of various cytosolic labeled metabolites using GC-MS analysis. Specifically, Aspartate at a mass of M+1 is a metabolite that comes out of the mitochondria via Citrin.

As shown in FIG. 15A, compound “3” (L-Glutamic acid γ-benzyl ester) inhibited Aspartate M+1 relative to control.

One of the expected outcomes of Aspartate M+1 inhibition is Uracil M+1 inhibition. Indeed, as shown in FIG. 15B compound “3” gave the highest Uracil M+1 inhibition among the tested compounds (˜35%). In addition, compounds “1” and “2” also inhibited Uracil M+1 and it is possible they inhibit Citrin too.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. Dimmock, D. P. et al. The role of molecular testing and enzyme analysis in the management of hypomorphic citrullinemia. Am J Med Genet A 146A, 2885-2890, doi:10.1002/ajmg.a.32527 (2008).
2. Dimmock, D. et al. Novel human pathological mutations. Gene symbol: ASS1. Disease: Citrullinaemia. Hum Genet 126, 342 (2009).
3. Long, Y. et al. Arginine deiminase resistance in melanoma cells is associated with metabolic reprogramming, glucose dependence, and glutamine addiction. Mol Cancer Ther 12, 2581-2590, doi:10.1158/1535-7163.MCT-13-0302 (2013).
4. Delage, B. et al. Arginine deprivation and argininosuccinate synthetase expression in the treatment of cancer. Int J Cancer 126, 2762-2772 (2010).
5. Huang, H. Y. et al. ASS1 as a novel tumor suppressor gene in myxofibrosarcomas: aberrant loss via epigenetic DNA methylation confers aggressive phenotypes, negative prognostic impact, and therapeutic relevance. Clin Cancer Res 19, 2861-2872, doi:10.1158/1078-0432.CCR-12-2641 (2013).
6. Kobayashi, E. et al. Reduced Argininosuccinate Synthetase Is a Predictive Biomarker for the Development of Pulmonary Metastasis in Patients with Osteosarcoma. Mol Cancer Ther (2010).
7. Allen, M. D. et al. Prognostic and therapeutic impact of argininosuccinate synthetase 1 control in bladder cancer as monitored longitudinally by PET imaging. Cancer Res 74, 896-907, doi:10.1158/0008-5472.CAN-13-1702 (2014).
8. Morris, S. M., Jr. Recent advances in arginine metabolism: roles and regulation of the arginases. Br J Pharmacol 157, 922-930 (2009).
9. Zheng, L. et al. Reversed argininosuccinate lyase activity in fumarate hydratase-deficient cancer cells. Cancer & metabolism 1, 12, doi:10.1186/2049-3002-1-12 (2013).
10. Adam, J. et al. A role for cytosolic fumarate hydratase in urea cycle metabolism and renal neoplasia. Cell reports 3, 1440-1448, doi:10.1016/j.celrep.2013.04.006 (2013).
11. Wheatley, D. N. Controlling cancer by restricting arginine availability—arginine-catabolizing enzymes as anticancer agents. Anti-cancer drugs 15, 825-833 (2004).
12. Tsai, W. B. et al. Resistance to arginine deiminase treatment in melanoma cells is associated with induced argininosuccinate synthetase expression involving c-Myc/HIF-1alpha/Sp4. Mol Cancer Ther 8, 3223-3233 (2009).
13. Syed, N. et al. Epigenetic status of argininosuccinate synthetase and argininosuccinate lyase modulates autophagy and cell death in glioblastoma. Cell death & disease 4, e458, doi:10.1038/cddis.2012.197 (2013).
14. Fairbanks, L. D., Bofill, M., Ruckemann, K. & Simmonds, H. A. Importance of ribonucleotide availability to proliferating T-lymphocytes from healthy humans. Disproportionate expansion of pyrimidine pools and contrasting effects of de novo synthesis inhibitors. J Biol Chem 270, 29682-29689 (1995).
15. Fajas, L. Metabolic control in cancer cells. Annales d'endocrinologie 74, 71-73, doi:10.1016/j.ando.2013.03.021 (2013).
16. Duarte, N. C. et al. Global reconstruction of the human metabolic network based on genomic and bibliomic data. Proc Natl Acad Sci USA 104, 1777-1782, doi:10.1073/pnas.0610772104 (2007).
17. Kobayashi, K., Saheki, T. & Song, Y. Z. in GeneReviews (eds R. A. Pagon et al.) (2005).
18. Marion, V. et al. Hepatic adaptation compensates inactivation of intestinal arginine biosynthesis in suckling mice. PLoS One 8, e67021, doi:10.1371/journal.pone.0067021 (2013).
19. Warburg, O. On the origin of cancer cells. Science 123, 309-314 (1956).
20. Yizhak, K. et al. Phenotype-based cell-specific metabolic modeling reveals metabolic liabilities of cancer. eLife 3, doi:10.7554/eLife.03641 (2014).
21. Kuijjer, M. L. et al. IR/IGF1R signaling as potential target for treatment of high-grade osteosarcoma. BMC cancer 13, 245, doi:10.1186/1471-2407-13-245 (2013).
22. Kabbarah, O. et al. Integrative genome comparison of primary and metastatic melanomas. PLoS One 5, e10770, doi:10.1371/journal.pone.0010770 (2010).
23. del Arco, A. et al. Expression of the aspartate/glutamate mitochondrial carriers aralar1 and citrin during development and in adult rat tissues. Eur J Biochem 269, 3313-3320 (2002).
24. Tan, G. S. et al. Novel proteomic biomarker panel for prediction of aggressive metastatic hepatocellular carcinoma relapse in surgically resectable patients. Journal of proteome research 13, 4833-4846, doi:10.1021/pr500229n (2014).
25. Robitaille, A. M. et al. Quantitative phosphoproteomics reveal mTORC1 activates de novo pyrimidine synthesis. Science 339, 1320-1323, doi:10.1126/science.1228771 (2013).
26. Ben-Sahra, I., Howell, J. J., Asara, J. M. & Manning, B. D. Stimulation of de novo pyrimidine synthesis by growth signaling through mTOR and S6K1. Science 339, 1323-1328, doi:10.1126/science.1228792 (2013).
27. Folger, O. et al. Predicting selective drug targets in cancer through metabolic networks. Molecular systems biology 7, 501, doi:10.1038/msb.2011.35 (2011).
28. Varma, A. P., B. O. Metabolic flux balancing: Basic concepts, scientific and practical use. Bio. Technology 12, 994-998 (1994).
29. Bordel, S., Agren, R. & Nielsen, J. Sampling the solution space in genome-scale metabolic networks reveals transcriptional regulation in key enzymes. PLoS computational biology 6, e1000859, doi:10.1371/journal.pcbi.1000859 (2010).
30. Lee, J. K. et al. A strategy for predicting the chemosensitivity of human cancers and its application to drug discovery. Proc Natl Acad Sci USA 104, 13086-13091, doi:10.1073/pnas.0610292104 (2007).
31. Itzkovitz, S. et al. Single-molecule transcript counting of stem-cell markers in the mouse intestine. Nat Cell Biol 14, 106-114, doi:10.1038/ncb2384 (2012).
32. Gu, G. J. et al. Protein tag-mediated conjugation of oligonucleotides to recombinant affinity binders for proximity ligation. New biotechnology 30, 144-152, doi:10.1016/j.nbt.2012.05.005 (2013).
Rabinovich, S. et al. Diversion of aspartate in ASS1-deficient tumours fosters de novo pyrimidine synthesis. Nature 527, 379-383, doi:10.1038/nature15529 (2015).

1. A method of treating cancer in a subject in need thereof, the method comprising administering to the subject a therapeutically effective amount of an agent which downregulates a mitochondrial aspartate-dependent pyrimidine synthesis, thereby treating the cancer.

2. A pharmaceutical composition comprising as an active ingredient a therapeutically effective amount of at least two distinct agents and a pharmaceutically acceptable carrier, wherein said at least two distinct agents selected:
down-regulating in tumor cells a mitochondrial aspartate-dependent pyrimidine synthesis; or
at least one of said at least two distinct agents down-regulating in tumor cells a mitochondrial aspartate-dependent pyrimidine synthesis and a second agent of said at least two distinct agents being an agent selected from the group consisting of: an agent for arginine depletion therapy, an agent for glutamine depletion, chemotherapy which inhibits production of nucleotide(s), an agent which inhibits the mammalian target of Rapamycin (mTOR) pathway, a thymidine synthase inhibitor, and an agent which over-expresses the Argininosuccinate synthase polypeptide.

3. (canceled)

4. A kit for treating cancer comprising at least two containers, said at least two containers separately packaging at least two distinct agents selected:
down-regulating in tumor cells a mitochondrial aspartate-dependent pyrimidine synthesis; or
at least one of said at least two distinct agents down-regulating in tumor cells a mitochondrial aspartate-dependent pyrimidine synthesis and a second agent of said at least two distinct agents being an agent selected from the group consisting of: an agent for arginine depletion therapy, an agent for glutamine depletion, chemotherapy which inhibits production of nucleotide(s), an agent which inhibits the mammalian target of Rapamycin (mTOR) pathway, a thymidine synthase inhibitor, and an agent which over-expresses the Argininosuccinate synthase polypeptide.

5. The method of claim 1, wherein said agent which downregulates a mitochondrial aspartate-dependent pyrimidine synthesis is an agent which downregulates activity or expression of a polypeptide or an enzyme selected from the group consisting of citrin, carbamoyl-phosphate synthase 2, aspartate transcarbamylase and dihydroorotase.

6. The method of claim 1, wherein said agent which downregulates a mitochondrial aspartate-dependent pyrimidine synthesis is an agent which downregulates activity or expression of citrin.

7. The method of claim 1, wherein said agent which downregulates a mitochondrial aspartate-dependent pyrimidine synthesis is an oligonucleotide.

8. The method of claim 1, wherein said agent which downregulates a mitochondrial aspartate-dependent pyrimidine synthesis is a small molecule.

9. The method of claim 7, wherein said oligonucleotide is selected from the group consisting of an RNA silencing agent and a genome editing agent.

10. The method of claim 1, wherein said agent which downregulates a mitochondrial aspartate-dependent pyrimidine synthesis comprises a plurality of agents for downregulating activity or expression of at least two of said citrin, carbamoyl-phosphate synthase 2, aspartate transcarbamylase and dihydroorotase.

11. The method of claim 1, further comprising administering to the subject an arginine depletion therapy.

12. The method of claim 1, further comprising administering to the subject an agent for glutamine depletion.

13. The method of claim 1, further comprising administering to the subject chemotherapy which inhibits production of nucleotides.

14. The method of claim 13, wherein said chemotherapy comprises pyrimidine analog(s), purine analog(s) and/or folate antagonist(s).

15. The method of claim 1, further comprising administering to the subject an agent which inhibits the mammalian target of Rapamycin (mTOR) pathway.

16. The method of claim 15, wherein said agent is Rapamycin, rapalogs (rapamycin derivatives) and/or mTORC1/mTORC2.

17. The method of claim 1, further comprising administering to the subject a thymidine synthase inhibitor.

18. The method of claim 17, wherein said thymidine synthase inhibitor is Fluorouracil (5-FU).

19. The method of claim 1, further comprising over-expressing within tumor cells of the subject the Argininosuccinate synthase polypeptide.

20. The method of claim 1, wherein the cancer is characterized by an increased level of citrin as compared to the level of said citrin in a non-malignant tissue of the same origin as said cancer.

21. The method of claim 1, wherein the cancer is characterized by a decreased level of Argininosuccinate synthase as compared to the level of said Argininosuccinate synthase in a non-malignant tissue of the same origin as said cancer.
